(19) United States
(12) Patent Application Publication
(10) Pub. No.: US 2014/0343001 A1
(43) Pub. Date: Nov. 20, 2014

(54) INDOLE DERIVATIVES INHIBITORS OF ENZYME LACTATE DEHYDROGENASE (LDH)

(71) Applicant: Universitá di Pisa, Pisa (PI) (IT)

(72) Inventors: Filippo Minutolo, Pisa (PI) (IT); Marco Macchia, Livorno (LI) (IT); Carlotta Granchi, Pontedera (PI) (IT); Valeria Di Bussolo, Pisa (PI) (IT); Gino Giannaccini, Forte dei Marmi (LU) (IT); Antonio Lucacchini, Forte dei Marmi (LU) (IT); Paul J. Hergenrother, Champaign, IL (US); Emilia C. Calvaresi, Champaign, IL (US)

(73) Assignee: Universita di Pisa, Pisa (PI) (IT)

(21) Appl. No.: 14/366,852

(22) PCT Filed: Dec. 19, 2012
(86) PCT No.: PCT/EP2012/076221

§ 371 Date: Jun. 19, 2014

(30) Foreign Application Priority Data
Dec. 20, 2011 (IT) PI2011A000143

(51) Int. Cl. C07H 17/02; C07D 209/42; C07H 1/00

(52) U.S. Cl. CPC C07H 17/02; C07H 1/00; C07D 209/42
USPC 514/27; 536/17.4; 548/492; 514/419; 546/201; 514/323

(57) ABSTRACT

The present invention encompasses compounds having general formula (I) able to inhibit the lactate production (lactic acid) involved in the angiogenesis of tumoral tissues, in the glycolytic metabolic process of tumoral cells, of immune system cells in asthmatic diseases, in vascular cells in the pulmonary hypertension, in the treatment of chronic back pain or hyperoxaluria, and in the process by which the parasites protozoan causing malaria obtain most of the necessary energy.

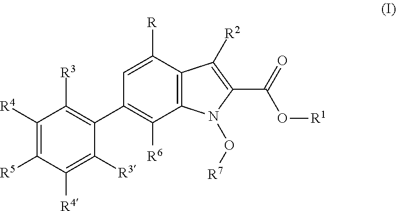

### FIELD OF INVENTION

The present invention relates to compounds able to inhibit the production of lactate (lactic acid) involved in angiogenesis of cancer tissues, as well as in the glycolytic metabolic process of cancer cells, of cells of the immune system in asthmatic diseases, of vascular cells in pulmonary hypertension, and in the process through which the protozoan parasites causing malaria get most of their required energy.

### BACKGROUND OF INVENTION

Almost a century ago, Otto Warburg described for the first time the importance of the relationship between cancer diseases and the alteration of cellular metabolism [Warburg, O. The metabolism of tumors in the body. J. January Physiol. 1927, 8, 519-530; Warburg, O. On the origin of cancer cells. Science 1956, 123, 309-314], indicating glycolysis as the main metabolic pathway of anaerobic metabolism of glucose in cancer cells [Koppenol, W. H., Bounds, P. L. Dang, C. V. Nat. Rev. Cancer 2011, 11, 325]. These cells are more “starved” of nutrients compared to normal cells, in order to maintain their high levels of proliferation. The so-called Warburg effect, which manifests itself in the majority of invasive tumor phenotypes, consists of a shift from the metabolic oxidative phosphorylation (OXPHOS) towards an increased anaerobic glycolysis. This change is accompanied by: 1) a higher consumption of glucose, due to the low efficiency in energy production by anaerobic glycolysis; 2) an increased extracellular acidosis, due to the large production of lactic acid and other acids. This change ensures adequate metabolic energy production from glucose and, consequently, a high viability even in the absence of sufficient levels of oxygen in the hypoxic regions of cancer tissues [Cairns, R. A., Harris, I. S., Mak, T. W. Nat. Rev. Cancer 2011, 11, 85], which are particularly invasive and susceptible to metastases.

In addition, hypoxic tumors show a high resistance against traditional therapeutic treatments such as radiation therapy and chemotherapy. The hypoxic tumor radioresistance is mainly due to the low tendency to form oxygen-dependent cytotoxic radicals as a result of irradiation; resistance to chemotherapy is essentially due to limited blood supply and the low proliferation rate, while most of the currently used chemotherapeutic treatments target rapidly dividing cells.

Therefore, for the treatment of hypoxic tumors, alternative routes to the traditional ones have been sought. In particular, compounds capable of interfering with the main mechanisms used by tumoral cells for their growth and proliferation are currently studied for the treatment of hypoxic tumors.

For example, a group of prodrugs exploits the reducing environment of hypoxic tumors for their activation [Brown, J. M. Wilson, W. R. Nat. Rev. Cancer 2004, 4, 437-447; Patterson, A. V. et al., Clin. Cancer Res 2007, 13, 3922-3932; Duan, J.-X. et al., J. Med. Chem. 2008, 51, 2412-2420] and one such example is tirapazamine. This is a benzotriazine able to release cytotoxic radicals when activated in the hypoxic environment. However, this prodrug has a reduced capacity of penetration into the tumoral mass. Other prodrugs of this type have been used for the treatment of hypoxic tumors, but with mixed results.

A particularly interesting feature of cancer cells is their high glycolytic activity, greater than 200 times compared to healthy cells [Gatenby, R. A.; Gillies, R. J. Nat. Rev. Cancer 2004, 4, 891-899; Vander Heiden, M. G., Cantley, L. C., Thompson, C. B. Science 2009, 324, 1029-1033]. This is due, on the one hand, to the high local consumption of oxygen that generates a shortage of oxygen resulting in increased levels of glycolysis, and on the other hand to the presence in higher quantities of a particular form of hexokinase bound to the mitochondria, which generates an increased glycolytic activity without the oxygen being necessarily consumed (Warburg Effect). Glycolysis is a metabolic process by which one glucose molecule is transformed into two molecules of pyruvate with the concomitant generation of 2 molecules of ATP (the energy currency of the cell) and 2 molecules of NADH (nicotinamide adenine reduced dinucleotide).

Glycolysis comprises ten reactions that occur in the cytoplasm of cells and which are catalyzed by specific enzymes, including hexokinases, phosphoglucose isomerases, aldolases and pyruvate kinases. The process is catabolic, as complex and energetic molecules are transformed into simple and less energetic molecules, resulting in the accumulation of energy.

Glycolysis can be performed both in aerobic conditions, i.e. in the presence of oxygen, and under anaerobic conditions, i.e. in the absence of oxygen. In both cases, one mole of glucose generates two moles of ATP, 2 moles of NADH and two moles of pyruvate. In the presence of oxygen, the molecules of pyruvate produced by glycolysis are transported within the mitochondrial matrix, where they are decarboxylated and then enter in the Krebs cycle, the tricarboxylic acid cycle, and are then degraded to carbon dioxide and water with the subsequent generation of ATP by oxidative phosphorylation.

Under anaerobic conditions, the molecules of pyruvic acid are reduced to lactic acid or lactate. This reaction is catalyzed by the enzyme lactate dehydrogenase (LDH).

The majority of invasive tumor phenotypes, including the haematological ones, such as leukemias, show a net metabolic change from oxidative phosphorylation to anaerobic glycolysis. This ensures a sufficient supply of energy and anabolic nutrients to sustain tumour growth. The particular metabolism of cancer cells led to a novel therapeutic approach against cancer that involves the search for molecules able to inhibit a given enzyme among those involved in the reactions of glycolysis [Kroemer, G.; Pouyssegur, J. Cancer Cell 2008, 13, 472-482]. Inhibition of one of the reactions involved in the mechanism of glycolysis would, in fact, stop the process by which cancer cells generate the energy necessary to sustain their spread and survival [Porporato, P. E.; Dhup, S., Dadhich, R. K. Copetti, T.; Sonveaux, P. Front. Pharmacol. 2011, 2, 49; Scatena, R., Bottoni, P., Pontoglio, A.; Mastrototaro, L., Giardina, B. Expert Opin. Investig. Drugs 2008, 17, 1533-1545; Sheng, H., Niu, B., Sun, H. Curr. Med. Chem. 2009, 16, 1561-1587; Sattler, U. G. A.; Hirschhaeuser, F., Mueller-Klieser, W. F. Curr. Med. Chem. 2010, 17, 96-108; Tennant, D. A., Durán, R. V., Gottlieb, E. Nat. Rev. Cancer 2010, 10, 267-277].

A molecule widely studied, because it was considered able to interfere with the glycolysis of the tumor cells, is lonidamine, an inhibitor of the enzyme hexokinase (HK) [Price, G. S., Page, R. L. Riviere, J. E., Cline, J. M. Thrall, D. E. Cancer Chemother. Pharmacol. 1996, 38, 129-135.]. Hexokinase catalyzes the reaction of phosphorylation of intracellular glucose to glucose-6-phosphate with the consumption of a molecule of ATP. This is the first step of glycolysis and one of the three basic steps of the entire pathway, since the molecule of phosphorylated glucose, besides not being able to exit the cell membrane, destabilizes, becoming more susceptible to continue the catabolic pathway. However, lonidamine has not negligible side effects, in particular pancreatic toxicity and liver toxicity.

Another extensively studied inhibitor of the hexokinase (HK) is 2-deoxyglucose (2-DG). However, the studies conducted so far have shown a lack of efficacy for the treatment of hypoxic tumors [Maher, J. C.; Wangpaichitr, M.; Savaraj, N.; Kurtoglu, M.; Lampidis, T. J. Mol. Cancer. Ther. 2007, 6, 732-741].

Another inhibitor of HK is 3-bromopyruvate, but so far no clinical data are available for this compound [Ko, Y. H., Smith, B. L. Wang, Y., et al. Biochem. Biophys. Res Commun. 2004, 324, 269-275].

Another substance being studied for its ability to interfere with the glycolytic process is dichloroacetate (DCA), an inhibitor of the pyruvate dehydrogenase kinase (PDK, involved in the glycolysis) and currently in clinical trials [Bonnet, S., Archer, S. L.; Allalunis-Turner, J., et al. Cancer Cell 2007, 11, 37-51]. Lactate dehydrogenase (LDH) is one of the key enzymes involved in the peculiar carbohydrate metabolism of cancerous cells. As mentioned above, this enzyme catalyzes the reaction of reduction of pyruvate to lactate, using as cofactor NADH that is oxidized to NAD⁺.

In humans, the lactate dehydrogenase enzyme (LDH) is a tetrameric enzyme that can exist in 5 different isoforms (hLDH1-5), most of them localized in the cytosol. This enzyme is composed of two types of monomeric subunits, the LDH-A (or LDH-M, of muscles) and LDH-B (or LDH-H, of the heart) the combination of which gives rise to the following 5 tetrameric isoforms: hLDH1: LDH-B₄, hLDH2: LDH-AB₃, hLDH3: LDH-A₂B₂, hLDH4: LDH-A₃ Band hLDH5: LDH-A₄. Among these, the enzyme hLDH1 is predominantly present in the heart, while the hLDH5 predominantly in the liver and in skeletal muscles.

In the highly invasive hypoxic tumors, the hLDH5 isoform, consisting only of LDH-A subunits, is overexpressed and is induced by the hypoxia-induced factor, HIF-1α. Plasma levels of hLDH5 are not exclusively related to non-specific cellular damage, but can also be caused by an over-expression induced by malignant tumor phenotypes. Therefore, the levels of hLDH5 in serum and plasma can often be indicative of the presence cancer. The over-expression of LDH-A has been found in several tumor cell lines together with an overproduction of the glucose transporter GLUT1 following oxygen deprivation [Sørensen, B. S. et al., Radiother. Oncol. 2007, 83, 362-366]. In addition, the over-expression of LDH-A (and its tetrameric fully functional form, hLDH5) has been detected in many invasive and hypoxic cancerous forms [Koukorakis, M. I. et al., Clin. Experim. Metast. 2005, 22, 25-30; Koukorakis, M. I. et al., Cancer Sci 2006, 97, 1056-1060] and a strong correlation between this phenomenon and the stabilization and activity of HIF-1α has been found [Kolev, Y.; Uetake, H., Takagi, Y.; Sugihara, K. Ann. Surg. Oncol. 2008, 15, 2336-2344].

The lactic acid production in tumor tissues triggers a mechanism defined as the lactate “shuttle”, which involves an exchange of this metabolite between some tumoral cells (especially the hypoxic ones), which produce it through glycolysis, and other tumoral cells, including the endothelial ones, that promote the angiogenesis phenomenon [Sonveaux, P. et al. J. Clin. Invest. 2008, 118, 3930-3942; Draoui, N., Feron, O. Dis. Model. Mech. 2011, 4, 727-732; Hirschhaeuser, F., Sattler, U. G. A., Mueller-Klieser, W. Cancer Res 2011, 71, 6921-6925].

Based on these considerations, it is apparent that a reduced lactic acid production in cancerous tissues is expected to interfere in a synergistic way with many biochemical pathways that support the survival and proliferation of cancer cells, such as energy production, the formation of anabolites and angiogenesis.

Currently, LDH-A/hLDH5 is considered as one of the most promising new molecular targets for cancer therapy, since its suppression by shRNA in cells of invasive breast cancer (Neu4145) resulted in a significant decrease in invasiveness and tumor growth [Fantin, V. R., St-Pierre, J., Leder, P. Cancer Cell 2006, 9, 425-434]. Forecasts relating to the absence of any toxic effects related to a selective inhibition of LDH-A/hLDH5 may derive from the observation that some individuals with a hereditary deficiency of the gene for the LDH-A, show muscle damages (myopathy) only after an intense anaerobic effort, while do not have any particular symptoms under ordinary conditions [Kanno, T., Sudo, K., Maekawa, M. et al., Clin. Chim. Acta 1988, 173, 89-98; B. J. Lee, L. Zand, N. J. Manek, L. L. Hsiao, D. Babovic-Vuksanovic, M. E. Wylam, Q. Qian, Arthritis Care Res 2011, 63, 1782-1786].

Historically, the inhibition of LDH has been reported for pyruvic acid derivatives [Cooper, A. J. L., U.S. 4950602 (1990)], salicylates [Cheshire, R. M. Park, M. V. Int J. Biochem. 1977, 8, 637-643], cyclopropyl derivatives [Maclnnes, I.; Nonhebel, D. C.; Orszulik, S. T., Suckling, C. J. Wrigglesworth, R. J. Chem. Soc, Perkin Trans. 1 1983, 2771-2776], or for 17-β-estradial [Spellman, C. M. Fottrell, P. F. FEBS Lett 1972, 21, 186-188].

Examples, in which inhibition of LDH has an antitumoral effect in cell lines or in tumors, have been reported in relation to: cells of human lymphoma P493 and related murine xenografts [Le, A. et al. Proc Natl. Acad. Sci. U.S.A. 2010, 107, 2037-2042]; hepatocellular carcinoma cells HepG2 and PLC/PRF/5 [Fiume, L. et al. Pharmacology 2010, 86 (3), 157-162]; glioblastoma cells GS-2 and breast cancer MDA-MB-231 and related murine xenografts [Ward, C. S. et al. Cancer Res 2010, 70 (4), 1296-1305; Mazzio, E.; Soliman, K. WO2006017494]; breast cancer cells MDA-MD-435 resistant to taxol [Zhou, M. et al. Molecular Cancer 2010, 9, 33]; Dalton's lymphoma murine models [Koiri, R. K. et al. Invest. New Drugs 2009, 27, 503-516; Pathak, C.; Vinayak, M. Mol. Biol. Rep. 2005, 32, 191-196]; human tumor cell lines MCF (breast), KB (oral), KB-VIN (vincristine-resistant oral), SK-MEL-2 (melanoma), U87-MG (glioma), HCT-8 (colon), IA9 (ovarian cancer), A549 (alveolar adenocarcinoma) and PC-3 (prostate) [Mishra, L. et al. Indian J. Exp Biol. 2004, 42 (7), 660-666]; glioma cells U87MG and AI72, culture of tumoral cells from primary glioma “HTZ” [Baumann, F. et al. Neuro-Oncology 2009, 11 (4), 368-380]; cells of renal cancer (HLRCC) and alveolar adenocarcinoma (A549) [Xie, H. et al. Mol. Cancer. Ther. 2009, 8 (3), 626-635]; c-Myc-transformed fibroblasts Ratla, c-Myc-transformed human lymphoblastoid cells, and Burkitt lymphoma cells [Shim, H. et al. Proc Natl. Acad. Sci. U.S.A. 1997, 94, 6658-6663; Dang, C., Shim, H. WO9836774]; cells of Burkitt lymphoma EB2 [Willsmore, R. L. Waring, A. J. IRCS Medical Science: Library Compendium 1981, 9 (11), 1003-1004]; cells of colon adenocarcinoma HT29 and of malignant glioma U118MG [Goerlach, A. et al. Int J. Oncol. 1995, 7 (4), 831-839]; human glioma cell lines HS683, U373, U87 and U138, and rat glioma C6, SW-13 (adrenal gland), MCF-7 (breast), T47-D (breast), HeLa (cervical cancer), SK-MEL-3 (melanoma), Colo 201 (colon) and BRW (cell line derived from a patient with primitive neuroectodermal tumor) [Coyle, T. et al. J. Neuro-Oncol. 1994, 19 (1), 25-35].

Moreover, the production of lactate via glycolysis in T lymphocytes of the respiratory system plays a key role in the development of asthmatic diseases. Indeed, it has been shown that the glycolytic process is increased in asthma and that the inhibition of glycolysis hinders the activation of T lymphocytes and the development of asthma [Ostroukhova, M.; et al. Am J. Physiol.-Lung Cell Mol. Physiol. 2012, 302, L300-L307.]. In addition, it has been also reported that the metabolic change towards the glycolysis could be the cause of the increased resistance to apoptosis and of the increased proliferation of vascular cells, which characterize pulmonary hypertension [Tuder, R. M. Davis, L. A., Graham, B. B. Am J. Respir. Crit. Care Med., 2012, 185, 260-266]. Therefore, a reduction of glycolysis by inhibition of lactate production can be therapeutically advantageous also for the treatment of this pathology.

Finally, a further medical use of inhibitors of lactate production can be found in the field of antimalarial agents, because the protozoan parasites causing malaria, during a phase of the infection cycle, use the lactic fermentation to obtain most of their energy. Therefore compounds capable of attacking the malaria parasites and therefore of stopping the infection through inhibition of the enzyme lactate dehydrogenase expressed by these parasites, which presents a high level of homology to human isoforms are therefore under study [Turgut-Balik, D., et al., Biotechnol. Lett 2004, 26, 1051-1055]. Indeed, many of the LDH inhibitors developed so far were originally designed as novel anti-malarial agents [Granchi, C., et al. Curr. Med. Chem. 2010, 17, 672-697].

Another possible application of the LDH inhibitors is the treatment of tissue metaplasia and heterotopic ossification in the idiopathic arthrofibrosis after intervention of a knee total arthroplasty [Freeman, T. A., et al. Fibrogenesis Tissue Repair. 2010, 3, 17].

Furthermore, LDH inhibitors may be used in cosmetic preparations, since they are able to stimulate the proliferation of keratocytes and the biosynthesis of collagen in the skin [Bartolone, J. B., et al. US5595730 (1997)].

Compounds capable of inhibiting the isoform C of LDH may also be used as male contraceptives [Odet F, et al. Biol. Reprod. 2008, 79 (1), 26-34; Yu Y, et al. Biochem. Pharmacol. 2001, 62, 81-89].

Furthermore, there is some evidence of the relevance of LDH to the pathology of primary hyperoxaluria (Biochim. Biophys. Acta 2005, 1753, 209-216) or chronic back pain (US2012022425).

Some of the most efficient inhibitors of hLDH5 isoform are the naphthalen-1-carboxylic FX-11 derivative [Le, A.; Cooper, C. R., Gouw, A. M. Dinavahi, R. Maitra, A., Deck, L. M. Royer, R. E., Vander Jagt, D. L. Semenza, G. L. Dang, C. V. Proc Natl. Acad. Sci. USA, 2010, 107, 2037-2042], phenylbutyric acid containing a portion that mimics the adenosine of NADH [Moorhouse, A. D., et al. Chem. Commun. 2011, 47, 230-232], and the natural polyphenol galloflavin [Manerba, M.; et al. ChemMedChem 2011, 7, 311-317].

Some N-hydroxyindole-2-carboxylic acids (NHI) were previously discovered at the University of Pisa [Granchi, C., et al. J. Med. Chem. 2011, 54, 1599-1612; Granchi, C., et al. Med. Chem. Commun. 2011, 2, 638-643; Granchi, C., et al. Eur. J. Med. Chem. 2011, 46, 5398-5407; Granchi, C., et al. Bioorg. Med. Chem. Lett 2011, 21, 7331-7336; Granchi, C.; Minutolo, F. ChemMedChem. 2012, 7, 1318-1350]. WO2011054525 describes the N-hydroxyindole-2-carboxylic acids (NHI) as novel inhibitors of the enzyme lactate dehydrogenase (LDH). Some of these NHI derivatives have shown inhibitory activities on hLDH5, being competitive against both the cofactor (NADH) and the substrate (pyruvate), with Ki values in the range of 1-100 uM. Now the authors have discovered that compounds of general formula (I), described below, are highly potent inhibitors of LDH and useful in the therapy, in particular for the treatment of proliferative diseases, preferably cancer, asthmatic diseases, pulmonary hypertension, malaria, primary hyperoxaluria or chronic back pain.

### SUMMARY OF THE INVENTION

According to the present invention there are provided compounds for medical use, having the general formula (I):

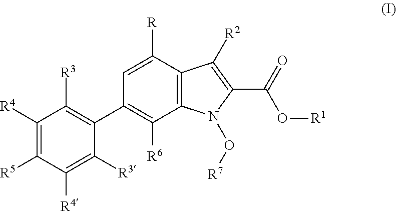

wherein:

R is selected from: F or CF₃;

R¹ is selected from: H; C₁-C₄ alkyl; C₁-C₄ alkyl substituted by phenyl, wherein the phenyl may optionally be substituted with one or more groups selected from halogen, nitro, methoxy, CF₃ or phenyl; C₁-C₄ alkyl substituted by C₃-C₇ cycloalkyl, wherein the C₃-C₇ cycloalkyl may optionally be substituted by C₁-C₄ alkyl; or piperidine, optionally substituted by C₁-C₄ alkyl or C₁-C₄ alkyl substituted by phenyl;

R² is selected from H, or CH₃;
R³, R⁴, R³′, R⁴′ and R⁵ are independently selected from H, Cl, or OCF₃;
R⁶ is selected from H, or C₆H₅;
R⁷ is selected from H, or

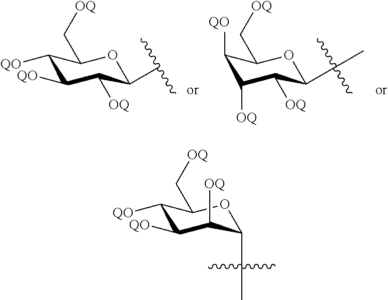

wherein Q is selected from H, or CH₃C(O); or a stereoisomer, tautomer, hydrate, solvate, or a pharmaceutically acceptable salt thereof; with the exclusion of the following compounds,

wherein R═CF₃ and:
R¹, R², R³, R⁴, R³′, R⁴′, R⁵, R⁶, and R⁷═H;
R¹, R², R³, R⁴, R³′, R⁴′, R⁵, and R⁷═H; R⁶═C₆H₅;
R¹, R², R³, R⁴, R³′, R⁴′, R⁵, R⁶, and R⁷═H; R⁵═Cl;
R¹, R³, R⁴, R³′, R⁴′, R⁵, R⁶ and R⁷═H; R²═CH₃;
R¹, R³, R⁴, R³′, R⁴′, R⁶, and R⁷═H; R²═CH₃; R⁵═Cl;
R¹, R², R³′, R⁴′, R⁴, R⁶, and R⁷═H; R³, R⁵═Cl.

In preferred embodiments compounds of general formula (I) have R selected from: F or CF₃.

In another embodiment R¹ is independently selected from H, CH₃, CH₂CH₃, CH(CH₃)₂, (CH₂)₃CH₃ or CH₂ (C₆H₅) or methyl substituted by a phenyl, wherein the phenyl may be unsubstituted or substituted by one or more groups selected from halogen, nitro, methoxy, CF₃ or phenyl; or piperidine N-substituted by CH₃ or CH₂ (C₆H₅); or 4-(tert-butyl)cyclohexyl.

In another embodiment R⁷ is H, R⁵ is Cl and R⁴, R⁴′ are independently H or Cl.

In another embodiment R⁷ is H and R³, R⁴, R³′, R⁴′ are independently H or Cl.

In another embodiment R⁷ is H and R³, R⁴, R³′, R⁴′, R⁵ are independently H or OCF₃.

In another embodiment R⁷ is

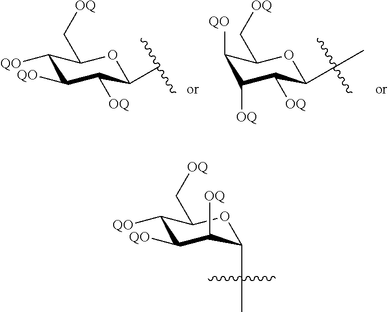

In another embodiment at least one between R¹ and R⁷ is different from hydrogen.

In a further preferred embodiment the compound of formula (I) for medical use is selected from the group consisting of:
ethyl 1-hydroxy-6-phenyl-4-(trifluoromethyl)-1H-indole-2-carboxylate (Example 1);
6-phenyl-1-(β-D-glucopyranosyl)oxy-4-(trifluoromethyl)-1H-indole-2-carboxylic acid (Example 2);
methyl 6-phenyl-1-(β-D-glucopyranosyl)oxy-4-(trifluoromethyl)-1H-indole-2-carboxylate (Example 3);
6-phenyl-1-(β-D-2,3,4,6-tetra-O-acetylglucopyranosyl)oxy-4-(trifluoromethyl)-1H-indole-2-carboxylic acid (Example 4);
methyl 6-phenyl-1-(β-D-2,3,4,6-tetra-O-acetylglucopyranosyl)oxy-4-(trifluoromethyl)-1H-indole-2-carboxylate (Example 5);
ethyl 6-phenyl-1-(β-D-glucopyranosyl)oxy-4-(trifluoromethyl)-1H-indole-2-carboxylate (Example 6);
ethyl 6-phenyl-1-(β-D-2,3,4,6-tetra-O-acetylglucopyranosyl)oxy-4-(trifluoromethyl)-1H-indole-2-carboxylate (Example 7);
methyl 6-(2,4-dichlorophenyl)-1-hydroxy-4-(trifluoromethyl)-1H-indole-2-carboxylate (Example 8);
ethyl 6-(2,4-dichlorophenyl)-1-hydroxy-4-(trifluoromethyl)-1H-indole-2-carboxylate (Example 9);
methyl 6-(2,4-dichlorophenyl)-1-(β-D-glucopyranosyl)oxy-4-(trifluoromethyl)-1H-indole-2-carboxylate (Example 10);
methyl 6-(2,4-dichlorophenyl)-1-(β-D-2,3,4,6-tetra-O-acetylglucopyranosyl)oxy-4-(trifluoromethyl)-1H-indole-2-carboxylate (Example 11);
ethyl 6-(2,4-dichlorophenyl)-1-(β-D-glucopyranosyl)oxy-4-(trifluoromethyl)-1H-indole-2-carboxylate (Example 12);
ethyl 6-(2,4-dichlorophenyl)-1-(β-D-2,3,4,6-tetra-O-acetylglucopyranosyl)oxy-4-(trifluoromethyl)-1H-indole-2-carboxylate (Example 13);
methyl 1-idroxy-6,7-diphenyl-4-(trifluoromethyl)-1H-indole-2-carboxylate (Example 14);
methyl 6-(4-clorophenyl)-1-hydroxy-4-(trifluoromethyl)-1H-indole-2-carboxylate (Example 15);
methyl 1-hydroxy-6-phenyl-3-methyl-4-(trifluoromethyl)-1H-indole-2-carboxylate (Example 16);
methyl 1-hydroxy-6-(4-clorophenyl)-3-methyl-4-(trifluoromethyl)-1H-indole-2-carboxylate (Example 17);
methyl 1-hydroxy-4-(trifluoromethyl)-6-(4-(trifluoromethoxy)phenyl)-1H-indole-2-carboxylate (Example 18);
methyl 1-hydroxy-4-(trifluoromethyl)-6-(3-(trifluoromethoxy)phenyl)-1H-indole-2-carboxylate (Example 19);
methyl 6-(2,4-dichlorophenyl)-1-hydroxy-3-methyl-4-(trifluoromethyl)-1H-indole-2-carboxylate (Example 20);
methyl 6-(3,4-dichlorophenyl)-1-hydroxy-4-(trifluoromethyl)-1H-indole-2-carboxylate (Example 21);
butyl 1-hydroxy-6-phenyl-4-(trifluoromethyl)-1H-indole-2-carboxylate (Example 22);
isopropyle 1-hydroxy-6-phenyl-4-(trifluoromethyl)-1H-indole-2-carboxylate (Example 23);
1-hydroxy-4-(trifluoromethyl)-6-(4-(trifluoromethoxy)phenyl)-1H-indole-2-carboxylic acid (Example 24);
1-hydroxy-4-(trifluoromethyl)-6-(3-(trifluoromethoxy)phenyl)-1H-indole-2-carboxylic acid (Example 25);
6-(2,4-dichlorophenyl)-1-hydroxy-3-methyl-4-(trifluoromethyl)-1H-indole-2-carboxilic acid (Example 26);
6-(3,4-dichlorophenyl)-1-hydroxy-4-(trifluoromethyl)-1H-indole-2-carboxilic acid (Example 27);
butyl 6-phenyl-1-(β-D-2,3,4,6-tetra-O-acetylglucopyranosyl)oxy-4-(trifluoromethyl)-1H-indole-2-carboxylate (Example 28);
butyl 1-(β-D-glucopyranosyl)oxy-6-phenyl-4-(trifluoromethyl)-1H-indole-2-carboxylate (Example 29);
isopropyl 6-phenyl-1-(β-D-2,3,4,6-tetra-O-acetylglucopyranosyl)oxy-4-(trifluoromethyl)-1H-indole-2-carboxylate (Example 30);
isopropyl 1-(β-D-glucopyranosyl)oxy-6-phenyl-4-(trifluoromethyl)-1H-indole-2-carboxylate (Example 31);
isopropyl 6-(2,4-dichlorophenyl)-1-hydroxy-4-(trifluoromethyl)-1H-indole-2-carboxylate (Example 32);
butyl 6-(2,4-dichlorophenyl)-1-hydroxy-4-(trifluoromethyl)-1H-indole-2-carboxylate (Example 33);
methyl 1-hydroxy-6-(2-(trifluoromethoxy)phenyl)-4-(trifluoromethyl)-1H-indole-2-carboxylate (Example 34);
1-hydroxy-6-(2-(trifluoromethoxy)phenyl)-4-(trifluoromethyl)-1H-indole-2-carboxylic acid (Example 35);
methyl 6-(2,3-dichlorophenyl)-1-hydroxy-4-(trifluoromethyl)-1H-indole-2-carboxylate (Example 36);
6-(2,3-dichlorophenyl)-1-hydroxy-4-(trifluoromethyl)-1H-indole-2-carboxylic acid (Example 37);
methyl 6-(2,5-dichlorophenyl)-1-hydroxy-4-(trifluoromethyl)-1H-indole-2-carboxylate (Example 38);
6-(2,5-dichlorophenyl)-1-hydroxy-4-(trifluoromethyl)-1H-indole-2-carboxylic acid (Example 39);
methyl 1-(β-D-gulopyranosyl)oxy-6-phenyl-4-(trifluoromethyl)-1H-indole-2-carboxylate (Example 40);
methyl 1-(α-D-mannopyranosyl)oxy-6-phenyl-4-(trifluoromethyl)-1H-indole-2-carboxylate (Example 41);
methyl 6-(3,5-dichlorophenyl)-1-hydroxy-4-(trifluoromethyl)-1H-indole-2-carboxylate (Example 42);
6-(3,5-dichlorophenyl)-1-hydroxy-4-(trifluoromethyl)-1H-indole-2-carboxylic acid (Example 43);
isopropyl 6-(2,4-dichlorophenyl)-1-(β-D-2,3,4,6-tetra-O-acetylglucopyranosyl)oxy-4-(trifluoromethyl)-1H-indole-2-carboxylate (Example 44);
isopropyl 6-(2,4-dichlorophenyl)-1-(β-D-glucopyranosyl)oxy-4-(trifluoromethyl)-1H-indole-2-carboxylate (Example 45);
butyl 6-(2,4-dichlorophenyl)-1-(β-D-2,3,4,6-tetra-O-acetylglucopyranosyl)oxy-4-(trifluoromethyl)-1H-indole-2-carboxylate (Example 46);
butyl 6-(2,4-dichlorophenyl)-1-(β-D-glucopyranosyl)oxy-4-(trifluoromethyl)-1H-indole-2-carboxylate (Example 47);
benzyl 1-hydroxy-6-phenyl-4-(trifluoromethyl)-1H-indole-2-carboxylate (Example 48);
4-(tert-butyl)cyclohexyl 1-hydroxy-6-phenyl-4-(trifluoromethyl)-1H-indole-2-carboxylate (Example 49);
4-(tert-butyl)cyclohexyl 6-(2,4-dichlorophenyl)-1-hydroxy-4-(trifluoromethyl)-1H-indole-2-carboxylate (Example 50);
methyl 4-fluoro-1-hydroxy-6-phenyl-1H-indole-2-carboxylate (Example 51);
4-fluoro-1-hydroxy-6-phenyl-1H-indole-2-carboxylic acid (Example 52);
methyl 6-(2,4-dichlorophenyl)-4-fluoro-1-hydroxy-1H-indole-2-carboxylate (Example 53);
6-(2,4-dichlorophenyl)-4-fluoro-1-hydroxy-1H-indole-2-carboxylic acid (Example 54);
benzyl 6-(2,4-dichlorophenyl)-1-hydroxy-4-(trifluoromethyl)-1H-indole-2-carboxylate (Example 55);
[1,1′-biphenyl]-4-ylmethyl 6-(2,4-dichlorophenyl)-1-hydroxy-4-(trifluoromethyl)-1H-indole-2-carboxylate (Example 56);
1-methylpiperidin-4-yl 1-hydroxy-6-phenyl-4-(trifluoromethyl)-1H-indole-2-carboxylate (Example 57);
1-methylpiperidin-4-yl 6-(2,4-dichlorophenyl)-1-hydroxy-4-(trifluoromethyl)-1H-indole-2-carboxylate (Example 58);
1-benzylpiperidin-4-yl 6-(2,4-dichlorophenyl)-1-hydroxy-4-(trifluoromethyl)-1H-indole-2-carboxylate (Example 59);
4-methoxybenzyl 6-(2,4-dichlorophenyl)-1-hydroxy-4-(trifluoromethyl)-1H-indole-2-carboxylate (Example 60);
4-nitrobenzyl 6-(2,4-dichlorophenyl)-1-hydroxy-4-(trifluoromethyl)-1H-indole-2-carboxylate (Example 61);
4-fluorobenzyl 6-(2,4-dichlorophenyl)-1-hydroxy-4-(trifluoromethyl)-1H-indole-2-carboxylate (Example 62);
4-chlorobenzyl 6-(2,4-dichlorophenyl)-1-hydroxy-4-(trifluoromethyl)-1H-indole-2-carboxylate (Example 63);
4-(trifluoromethyl)benzyl 6-(2,4-dichlorophenyl)-1-hydroxy-4-(trifluoromethyl)-1H-indole-2-carboxylate (Example 64).

The compounds of formula (I) as described above or a stereoisomer, tautomer, hydrate, solvate, or pharmaceutically acceptable salt thereof can be employed for use in the treatment of cancer, preferably for the treatment of tumor diseases by inhibition of glycolytic metabolism, or the process of angiogenesis of tumor cells, in particular against cancer diseases such as lymphoma, hepatocellular carcinoma, pancreatic tumor, brain tumor, breast cancer, lung cancer, colon cancer, cervical cancer, prostate cancer, kidney cancer, osteosarcoma, nasopharyngeal cancer, oral cavity cancer, melanoma, ovarian cancer. Most preferably the lung cancer is a non small cell lung carcinoma.

The compounds of formula (I) as described above or a stereoisomer, tautomer, hydrate, solvate, or pharmaceutically acceptable salt thereof can be employed for use in the treatment of asthma, pulmonary hypertension, idiopathic arthrofibrosis, malaria, chronic back, or of hyperoxaluria.

In particular, the compounds of formula (I) as described above can be used to produce drugs for the treatment of these pathologies.

It is another object of the invention a pharmaceutical composition characterized by comprising at least one compound as defined above or a stereoisomer, tautomer, hydrate, solvate, or pharmaceutically acceptable salt thereof and at least one pharmaceutically acceptable excipient and/or diluent.

It is another object of the invention a compound of general formula (I)

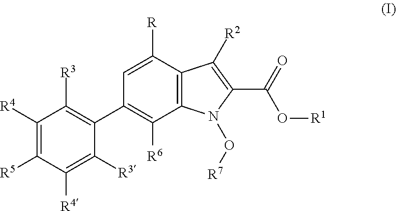

or a stereoisomer, tautomer, hydrate, solvate or a pharmaceutically acceptable salt of said compound,
wherein:
R is selected from: F or CF₃;
R¹ is selected from H; C₁-C₄ alkyl; C₁-C₄ alkyl substituted by a phenyl, wherein the phenyl may be optionally substituted by one or more groups selected from halogen, nitro, methoxy, CF₃ or phenyl; C₁-C₄ alkyl substituted by C₃-C₇ cycloalkyl, wherein the C₃-C₇ cycloalkyl is optionally substituted by C₁-C₄ alkyl; or piperidine, optionally substituted by C₁-C₄ alkyl or C₁-C₄ alkyl substituted by phenyl;
R² is selected from H or CH₃;
R³, R⁴, R³′, R⁴′ and R⁵ are independently selected from H, Cl, or OCF₃;
R⁶ is selected from H, or C₆H₅;
R⁵ is selected from H, or

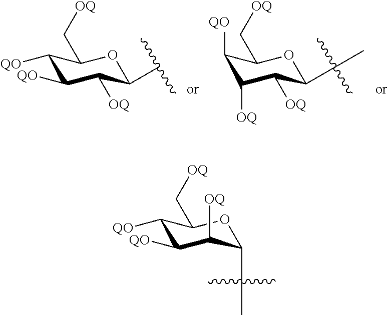

wherein Q is selected from H or CH₃C(O);

with the exclusion of the following compounds,

wherein R═CF₃ and:

### R¹, R², R³, R⁴, R³′, R⁴′, R⁵, R⁶ and R⁷═H;

R¹, R², R³, R⁴, R³′, R⁴′, R⁵, and R⁷═H; R⁶═C₆H₅;

### R¹, R², R³, R⁴, R³′, R⁴′, R⁶, and R⁷═H; R⁵═Cl;

R¹, R³, R⁴, R³′, R⁴′, R⁵, R⁶ and R⁷═H; R²═CH₃;
R¹, R³, R⁴, R³′, R⁴′, R⁶, and R⁷═H; R²═CH₃; R⁵═Cl;

### R¹, R², R⁴, R³′, R⁴′, R⁶, and R⁷═H; R³, R⁵═Cl;

R¹═CH₃; R², R³, R⁴, R³′, R⁴′, R⁵, R⁶ and R⁷═H;
R¹═CH₃; R², R³, R⁴, R³′, R⁴′, R⁵, and R⁷═H; R⁶═C₆H₅;
R¹═CH₃; R², R³, R⁴, R³′, R⁴′, R⁶, and R⁷═H; R⁵═Cl;
R¹═CH₃; R³, R⁴, R³′, R⁴′, R⁵, R⁶ and R⁷═H; R²═CH₃;
R¹═CH₃; R³, R⁴, R³′, R⁴′, R⁶, and R⁷═H; R²═CH₃; R⁵═Cl;
R¹═CH₃; R², R⁴, R³′, R⁴′, R⁶, and R⁷═H; R³, R⁵═Cl.

In a preferred embodiment at least one between R¹ and R⁷ is different from hydrogen.

In a further preferred embodiment the compound is selected from the group consisting of:
ethyl 1-hydroxy-6-phenyl-4-(trifluoromethyl)-1H-indole-2-carboxylate (Example 1);
6-phenyl-1-(β-D-glucopyranosyl)oxy-4-(trifluoromethyl)-1H-indole-2-carboxylic acid (Example 2);
methyl 6-phenyl-1-(β-D-glucopyranosyl)oxy-4-(trifluoromethyl)-1H-indole-2-carboxylate (Example 3);
6-phenyl-1-(β-D-2,3,4,6-tetra-O-acetylglucopyranosyl)oxy-4-(trifluoromethyl)-1H-indole-2-carboxylic acid (Example 4);
methyl 6-phenyl-1-(β-D-2,3,4,6-tetra-O-acetylglucopyranosyl)oxy-4-(trifluoromethyl)-1H-indole-2-carboxylate (Example 5);
ethyl 6-phenyl-1-(β-D-glucopyranosyl)oxy-4-(trifluoromethyl)-1H-indole-2-carboxylate (Example 6);
ethyl 6-phenyl-1-(β-D-2,3,4,6-tetra-O-acetylglucopyranosyl)oxy-4-(trifluoromethyl)-1H-indole-2-carboxylate (Example 7);
ethyl 6-(2,4-dichlorophenyl)-1-hydroxy-4-(trifluoromethyl)-1H-indole-2-carboxylate (Example 9);
methyl 6-(2,4-dichlorophenyl)-1-(β-D-glucopyranosyl)oxy-4-(trifluoromethyl)-1H-indole-2-carboxylate (Example 10);
methyl 6-(2,4-dichlorophenyl)-1-(β-D-2,3,4,6-tetra-O-acetylglucopyranosyl)oxy-4-(trifluoromethyl)-1H-indole-2-carboxylate (Example 11);
ethyl 6-(2,4-dichlorophenyl)-1-(β-D-glucopyranosyl)oxy-4-(trifluoromethyl)-1H-indole-2-carboxylate (Example 12);
ethyl 6-(2,4-dichlorophenyl)-1-(β-D-2,3,4,6-tetra-O-acetylglucopyranosyl)oxy-4-(trifluoromethyl)-1H-indole-2-carboxylate (Example 13);
methyl 1-idroxy-4-(trifluoromethyl)-6-(4-(trifluoromethoxy)phenyl)-1H-indole-2-carboxylate (Example 18);
methyl 1-idroxy-4-(trifluoromethyl)-6-(3-(trifluoromethoxy)phenyl)-1H-indole-2-carboxylate (Example 19);
methyl 6-(2,4-dichlorophenyl)-1-hydroxy-3-methyl-4-(trifluoromethyl)-1H-indole-2-carboxylate (Example 20);
methyl 6-(3,4-dichlorophenyl)-1-hydroxy-4-(trifluoromethyl)-1H-indole-2-carboxylate (Example 21);
butyl 1-hydroxy-6-phenyl-4-(trifluoromethyl)-1H-indole-2-carboxylate (Example 22);
isopropyl 1-hydroxy-6-phenyl-4-(trifluoromethyl)-1H-indole-2-carboxylate (Example 23);
1-hydroxy-4-(trifluoromethyl)-6-(4-(trifluoromethoxy)phenyl)-1H-indole-2-carboxylic acid (Example 24);
1-hydroxy-4-(trifluoromethyl)-6-(3-(trifluoromethoxy)phenyl)-1H-indole-2-carboxylic acid (Example 25);
6-(2,4-dichlorophenyl)-1-hydroxy-3-methyl-4-(trifluoromethyl)-1H-indole-2-carboxylic acid (Example 26);
6-(3,4-dichlorophenyl)-1-hydroxy-4-(trifluoromethyl)-1H-indole-2-carboxylic acid (Example 27);
butyl 6-phenyl-1-(β-D-2,3,4,6-tetra-O-acetylglucopyranosyl)oxy-4-(trifluoromethyl)-1H-indole-2-carboxylate (Example 28);
butyl 1-(β-D-glucopyranosyl)oxy-6-phenyl-4-(trifluoromethyl)-1H-indole-2-carboxylate (Example 29);
butyl 1-(β-D-2,3,4,6-tetra-O-acetylglucopyranosyl)oxy-4-(trifluoromethyl)-1H-indole-2-carboxylate (Example 30);

isopropyl 1-(β-D-glucopyranosyl)oxy-6-phenyl-4-(trifluoromethyl)-1H-indole-2-carboxylate (Example 31);
isopropyl 6-(2,4-dichlorophenyl)-1-hydroxy-4-(trifluoromethyl)-1H-indole-2-carboxylate (Example 32);
butyl 6-(2,4-dichlorophenyl)-1-hydroxy-4-(trifluoromethyl)-1H-indole-2-carboxylate (Example 33);
methyl 1-hydroxy-6-(2-(trifluoromethoxy)phenyl)-4-(trifluoromethyl)-1H-indole-2-carboxylate (Example 34);
1-hydroxy-6-(2-(trifluoromethoxy)phenyl)-4-(trifluoromethyl)-1H-indole-2-carboxylic acid (Example 35);
methyl 6-(2,3-dichlorophenyl)-1-hydroxy-4-(trifluoromethyl)-1H-indole-2-carboxylate (Example 36);
6-(2,3-dichlorophenyl)-1-hydroxy-4-(trifluoromethyl)-1H-indole-2-carboxylic acid (Example 37);
methyl 6-(2,5-dichlorophenyl)-1-hydroxy-4-(trifluoromethyl)-1H-indole-2-carboxylate (Example 38);
6-(2,5-dichlorophenyl)-1-hydroxy-4-(trifluoromethyl)-1H-indole-2-carboxylic acid (Example 39);
methyl 1-(β-D-gulopyranosyl)oxy-6-phenyl-4-(trifluoromethyl)-1H-indole-2-carboxylate (Example 40);
methyl 1-(α-D-mannopyranosyl)oxy-6-phenyl-4-(trifluoromethyl)-1H-indole-2-carboxylate (Example 41);
methyl 6-(3,5-dichlorophenyl)-1-hydroxy-4-(trifluoromethyl)-1H-indole-2-carboxylate (Example 42);
6-(3,5-dichlorophenyl)-1-hydroxy-4-(trifluoromethyl)-1H-indole-2-carboxylic acid (Example 43);
isopropyl 6-(2,4-dichlorophenyl)-1-(β-D-2,3,4,6-tetra-O-acetylglucopyranosyl)oxy-4-(trifluoromethyl)-1H-indole-2-carboxylate (Example 44);
isopropyl 6-(2,4-dichlorophenyl)-1-(β-D-glucopyranosyl)oxy-4-(trifluoromethyl)-1H-indole-2-carboxylate (Example 45);
butyl 6-(2,4-dichlorophenyl)-1-(β-D-2,3,4,6-tetra-O-acetylglucopyranosyl)oxy-4-(trifluoromethyl)-1H-indole-2-carboxylate (Example 46);
butyl 6-(2,4-dichlorophenyl)-1-(β-D-glucopyranosyl)oxy-4-(trifluoromethyl)-1H-indole-2-carboxylate (Example 47);
benzyl 1-hydroxy-6-phenyl-4-(trifluoromethyl)-1H-indole-2-carboxylate (Example 48);
4-(tert-butyl)cyclohexyl 1-hydroxy-6-phenyl-4-(trifluoromethyl)-1H-indole-2-carboxylate (Example 49);
4-(tert-butyl)cyclohexyl 6-(2,4-dichlorophenyl)-1-hydroxy-4-(trifluoromethyl)-1H-indole-2-carboxylate (Example 50);
methyl 4-fluoro-1-hydroxy-6-phenyl-1H-indole-2-carboxylate (Example 51);
4-fluoro-1-hydroxy-6-phenyl-1H-indole-2-carboxylic acid (Example 52);
methyl 6-(2,4-dichlorophenyl)-4-fluoro-1-hydroxy-1H-indole-2-carboxylate (Example 53);
6-(2,4-dichlorophenyl)-4-fluoro-1-hydroxy-1H-indole-2-carboxylic acid (Example 54);
benzyl 6-(2,4-dichlorophenyl)-1-hydroxy-4-(trifluoromethyl)-1H-indole-2-carboxylate (Example 55);
[1,1′-biphenyl]-4-ylmethyl 6-(2,4-dichlorophenyl)-1-hydroxy-4-(trifluoromethyl)-1H-indole-2-carboxylate (Example 56);
1-methylpiperidin-4-yl 1-hydroxy-6-phenyl-4-(trifluoromethyl)-1H-indole-2-carboxylate (Example 57);
1-methylpiperidin-4-yl 6-(2,4-dichlorophenyl)-1-hydroxy-4-(trifluoromethyl)-1H-indole-2-carboxylate (Example 58);
1-benzylpiperidin-4-yl 6-(2,4-dichlorophenyl)-1-hydroxy-4-(trifluoromethyl)-1H-indole-2-carboxylate (Example 59);
4-methoxybenzyl 6-(2,4-dichlorophenyl)-1-hydroxy-4-(trifluoromethyl)-1H-indole-2-carboxylate (Example 60);
4-nitrobenzyl 6-(2,4-dichlorophenyl)-1-hydroxy-4-(trifluoromethyl)-1H-indole-2-carboxylate (Example 61);
4-fluorobenzyl 6-(2,4-dichlorophenyl)-1-hydroxy-4-(trifluoromethyl)-1H-indole-2-carboxylate (Example 62);
4-chlorobenzyl 6-(2,4-dichlorophenyl)-1-hydroxy-4-(trifluoromethyl)-1H-indole-2-carboxylate (Example 63);
4-(trifluoromethyl)benzyl 6-(2,4-dichlorophenyl)-1-hydroxy-4-(trifluoromethyl)-1H-indole-2-carboxylate (Example 64).

Pharmaceutically acceptable salts comprise conventional non-toxic salts obtained by salification of a compound of formula (I). Pharmaceutically acceptable salts include, but are not limited to ammonium salts, alkaline metal salts, in particular sodium and potassium salts, alkaline earth metals salts, in particularly calcium and magnesium salts, and organic base salts such as dicyclohexylamine, morpholine, thiomorpholine, piperidine, pyrrolidine, short chain mono-, di- or trialkylamines such as ethyl-, t-butyl, diethyl-, di-isopropyl, triethyl, tributyl or dimethylpropylamine, or short chain mono-, di- or trihydroxyalkylamines such as mono-, di-, or trihydroxyethylamine. The invention includes within its scope all possible stoichiometric and non-stoichiometric forms of the salts of the compounds of formula (I).

Other pharmaceutically acceptable salts can be internal salts of compounds of formula (I), also known as zwitterions, where the molecule has regions of both negative and positive charge.

The compounds of formula (I) may exist in unsolvated as well as in solvated forms with pharmaceutically acceptable solvents such as water, EtOH and the like.

The skilled person in the art knows that many compounds may form complexes together with the solvents in which they are dissolved or precipitated or crystallised from. The complexes are known as solvates. For example, a complex with water is called a hydrate.

Based on the biological activity of the compounds of formula (I) in reducing the cellular production of lactate through inhibition of glycolysis, in particular at the level of the activity of the enzyme LDH, a compound included in the present invention may be used for the treatment of diseases, in which a reduction of lactate production is beneficial. These pathological conditions may be selected from the list of the various types of cancer, in particular lymphoma, hepatocellular carcinoma, pancreatic cancer, brain tumor, breast cancer, lung cancer, colon cancer, cervical cancer, prostate cancer, kidney cancer, osteosarcoma, nasopharyngeal cancer, oral cancer, melanoma and ovarian cancer. In addition, these conditions may include asthma, pulmonary hypertension, malaria and idiopathic arthrofibrosis, chronic back pain or hyperoxaluria.

The pharmaceutical compositions of the invention comprise a pharmaceutically acceptable carrier and/or excipients and/or pharmaceutically acceptable auxiliary substance. The pharmaceutical preparations can be administered orally, e.g. in the form of tablets, coated tablets, dragees, hard and soft gelatine capsules, solutions, emulsions or suspensions. The administration can also be effected rectally, e.g. in the form of suppositories, or topically, e.g. in the form of aerosol, or parenterally, e.g. in the form of injectable solutions.

The compounds of the invention can be processed with pharmaceutically inert carriers and/or excipients, inorganic or organic, for the production of pharmaceutical preparations. Lactose, corn starch or derivatives thereof, talc, stearic acids or its salts and similars can be used, for example, as carriers and/or excipients for the production of tablets, coated tablets, dragees and hard gelatine capsules. Suitable carriers for soft gelatine capsules are, for example, vegetable oils, waxes, fats, semi-solid or liquid polyols and similars. Depending on the nature of the active substance, no carriers may be required in the case of soft gelatine capsules. Excipients and/or carriers for the production of solutions and syrups are, for example, water, polyols, glycerol, vegetable oil and similars. Carriers and/or excipients for the production of suppositories are, for example, natural or hardened oils, waxes, fats, semi-liquid or liquid polyols and similars. The pharmaceutical preparations can, moreover, contain pharmaceutically acceptable auxiliary substances, such as preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Medicaments containing one or more compounds of the invention and therapeutically inert carrier and/or excipients are also an object of the present invention, as a process for their production, which includes the preparation comprising one or more compounds of the invention and, if desired, one or more other therapeutically valuable substances into a galenic formulation together with one or more therapeutically inert carriers and/or excipients.

The dosage can vary within wide limits and will have to be adjusted to the individual requirements in each particular case. In the case of oral administration the dosage for adults can vary from about 0.01 mg to about 1000 mg/kg body weight per day of a compound of the invention. The daily dosage may be administered as single dose or in divided doses and, in addition, the upper limit can also be exceeded, when this is found to be appropriate.

It is within the invention a method of treatment of cancer comprising administering in a subject in need thereof an effective amount of at least one compound as defined aboveor a stereoisomer, tautomer, hydrate, solvate, or pharmaceutically acceptable salt thereof.

In some embodiments, such pharmaceutical preparations, particularly those for the cure of cancer, may be administered in combination with other pharmaceutically active agents. The phrase “in combination”, as used herein, refers to agents that are simultaneously administered to a subject. It will be appreciated that two or more agents are considered to be administered “in combination” whenever a subject is simultaneously exposed to both (or more) of the pharmaceutically active agents. Each of the two or more agents may be administered according to different programs and schedules; it is not required that individual doses of different agents are administered at the same time, or in the same pharmaceutically composition. Rather, as long as both (or more) agents remain in the subject's body, they are considered to be administered “in combination”.

Non-exhaustive examples of suitable additional agents include:

a) antiproliferative/antineoplastic drugs and combinations thereof, as used in medical oncology, such as alkylating agents (for example platin derivatives like cis-platin, carboplatin, oxaliplatin, lobaplatin, satraplatin, nedaplatin, heptaplatin; nitrogen mustard such as chlorambucil, melphalan, chlormethine, cyclophosphamide, ifosfamide, trofosfamide, uramustine, bendamustine, estramustine; busulphan, temozolomide or nitrosoureas); antimetabolites (for example antifolates such as aminopterin, methotrexate, pemetrexed, raltitrexed); purines such as cladribine, clofarabine, fludarabine, mercaptopurine, pentostatin, thioguanine; pyrimidines like capecitabine, cytarabine, fluorouracil, floxuridine, gemcitabine; azacitidine, decitabine; cytosine arabinoside or hydroxyurea; antitumour antibiotics (for example anthracyclines like aclarubicin, amrubicin, daunomycin, doxorubicin, epirubicin, idarabicin, valrubicin, zorubicine; mitoxantrone; or antibiotics from streptomyces like actinomycin, bleomycin, mitomycin, or plicamycin); antimitotic agents (for example vinca alkaloids like vincristine, vinblastine, vindesine or vinorelbine; taxoids like docetaxel, paclitaxel or tesetaxel; epothilones like ixabepilone) and topoisomerase inhibitors (for example epipodophyllotoxins like etoposide and teniposide; amsacrine, camptothecin, irinotecan, rubitecan, and topotecan);
b) cytostatic agents such as antioestrogens (for example tamoxifen, toremifene, raloxifene, droloxifene and idoxifene), oestrogen receptor down regulators (for example fulvestrant), antiandrogens (for example bicalutamide, flutamide, nilutamide, liarozole or cyproterone acetate), LHRH antagonists or LHRH agonists (for example goserelin, leuprorelin or buserelin), progestogens (for example megestrol acetate), aromatase inhibitors (for example as anastrozole, letrozole, vorazole and exemestane) and inhibitors of 5-alpha-reductase such as finasteride;
c) agents which inhibit cancer cell invasion (for example metalloproteinase inhibitors and inhibitors of urokinase plasminogen activator receptor function);
d) inhibitors of growth factor function, for example growth factor antibodies, growth factor receptor antibodies (for example the anti-erbb2 antibody trastuzumab, the anti-erbb1 antibody cetuximab and panitumumab, the anti IGF1R antibody figitumumab), farnesyl transferase inhibitors, MEK inhibitors, tyrosine kinase inhibitors and serine/threonine kinase inhibitors, for example enzastaurin, dasatinib, erlotinib, gefitinib, imatinib, lapatinib, nilotinib, sorafenib, sunitinib, regorafenib, everolimus, sirolimus or temsirolimus;
e) antiangiogenic agents such as those which inhibit the effects of vascular endothelial growth factor, for example the anti-vascular endothelial cell growth factor antibody bevacizumab [Avastin™], lenalidomide or thalidomide;
f) cell cycle inhibitors including for example CDK inhibitors (for example flavopiridol, roscovitine) and other inhibitors of cell cycle checkpoints; inhibitors of aurora kinase and other kinases involved in mitosis and cytokinesis regulation;
g) proteasome inhibitors (for example lactacystin, bortezomib, epoxomicin);
h) HSP90 inhibitors (for example 17-AAG, AT-13387, KOS-953, KOS-1022, CNF-1010, CNF-2024, IPI-504, IPI-926, SNX 5422, STA-9090, VER-52296, PU-H17 or XL-888);
i) histone deacetylase inhibitors (for example SAHA, PXD101, JNJ-16241199, JNJ-26481585, SB939, ITF-2357, LBH589, PCI-24781, valproic acid, butyric acid, MS-275, MGCD0103 or FK-228);
j) selective COX-2 inhibitors (for example celecoxib), or non selective NSAIDs (for example diclofenac, flurbiprofen, ibuprofen, ketoprofen, or naproxen).

In another aspect, a compound of general formula (I) can be used in combination with radiation therapy. In yet another aspect, a compound of general formula (I) may be administered in combination with standard chemotherapy combinations such as, but not restricted to, CMF (cyclophosphamide, methotrexate and 5-fluorouracil), CAF (cyclophosphamide, doxorubicin and 5-fluorouracil), AC (doxorubicin and cyclophosphamide), FEC (5-fluorouracil, epirubicin, and cyclophosphamide), ACT or ATC (doxorubicin, cyclophosphamide, and paclitaxel), or CMFP (cyclophosphamide, methotrexate, 5-fluorouracil and prednisone).

In some embodiments the compounds of the invention used in pharmaceutical compositions may be labelled to make them suitable as diagnostic agents.

In particular, the labelling may be effected by the introduction of:
a radionuclide;
a fluorophore;
a ferromagnetic element;
an hyper-polarized atom (for example an hyper-polarized ¹³C for nuclear magnetic resonance techniques or NMR);
a combination thereof.

In addition, some of the atoms that form the compound of the present invention can be used as markers in combination with the appropriate diagnostic techniques, as for example the most abundant natural isotope of the fluorine (¹⁹F) in the case of use of nuclear magnetic resonance techniques (NMR).

Terms not specifically defined herein should be given the meanings that would be given to them by a person skilled in the field of the present invention. However, as indicated in the specification and appended claims, unless the contrary is specified, the following terms have the meaning indicated below.

The term “C₁-C₄ alkyl” encompasses a saturated hydrocarbon chain having one to four carbon atoms, being linear or branched. Examples of alkyl groups include methyl, ethyl, n-propyl, iso-propyl, n-butyl, tert-butyl, iso-butyl, sec-butyl. A “C₁-C₄ alkyl” is preferably methyl, ethyl, n-propyl, iso-propyl or tert-butyl.

The term “halogen” encompasses fluoro, chloro, bromo and iodo. Fluoro, chloro and bromo are particularly preferred.

The term “C₃-C₇-cycloalkyl” refers to a saturated hydrocarbon ring system having three to seven carbon atoms and zero heteroatoms. Suitable examples of C₃-C₇ cycloalkyl groups include cyclopropyl, cyclobutyl, cyclopentyl, cyclohexyl, and cycloheptyl, preferably cyclopentyl and cyclohexyl.

Whenever a chiral carbon is present in a chemical structure, it is intended that all stereoisomers associated with that chiral carbon are encompassed by the structure.

The compounds of formula (I) may exist in stereoisomeric forms (e.g. they may contain one or more asymmetric carbon atoms). The individual stereoisomers (enantiomers and diastereomers) and mixtures of these are included within the scope of the present invention. The present invention also covers the individual isomers of the compounds represented by formula (I) as mixtures with isomers thereof in which one or more chiral centres are inverted. The compounds of the invention may exist in tautomeric forms other than that shown in the formula and these are also included within the scope of the present invention.

In the present invention the term “effective amount” shall mean an amount which achieves a desired effect or therapeutic effect as such effect is understood by those of ordinary skill in the art.

Pharmaceutical compositions containing the molecules of the present invention may be manufactured by processes well known in the art, e.g., using a variety of well-known mixing, dissolving, granulating, levigating, emulsifying, encapsulating, entrapping or lyophilizing processes. The compositions may be formulated in conjunction with one or more physiologically acceptable carriers comprising excipients and auxiliaries which facilitate processing of the active compounds into preparations which can be used pharmaceutically. Proper formulation is dependent upon the route of administration chosen. Parenteral routes are preferred in many aspects of the invention.

For injection, including, without limitation, intravenous, intramusclular and subcutaneous injection, the compounds of the invention may be formulated in aqueous solutions, preferably in physiologically compatible buffers such as physiological saline buffer or polar solvents including, without limitation, a pyrrolidone or dimethylsulfoxide.

The compounds are preferably formulated for parenteral administration, e.g., by bolus injection or continuous infusion. Formulations for injection may be presented in unit dosage form, e.g. in ampoules or in multi-dose containers. Useful compositions include, without limitation, suspensions, solutions or emulsions in oily or aqueous vehicles, and may contain adjuncts such as suspending, stabilizing and/or dispersing agents. Pharmaceutical compositions for parenteral administration include aqueous solutions of a water soluble form, such as, without limitation, a salt of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances that increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions. Alternatively, the active ingredient may be in powder form for constitution with a suitable vehicle, e.g., sterile, pyrogen-free water, before use.

For oral administration, the compounds can be formulated by combining the active compounds with pharmaceutically acceptable carriers well-known in the art. Such carriers enable the compounds of the invention to be formulated as tablets, pills, lozenges, dragees, capsules, liquids, gels, syrups, pastes, slurries, solutions, suspensions, concentrated solutions and suspensions for diluting in the drinking water of a patient, premixes for dilution in the feed of a patient, and the like, for oral ingestion by a patient.

Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropyl-methylcellulose, sodium carboxy-methylcellulose, and/or polyvinylpyrrolidone (PVP). If desired, disintegrating agents may be added, such as cross-linked polyvinyl pyrrolidone, agar, or alginic acid. A salt such as sodium alginate may also be used.

For administration by inhalation, the compounds of the present invention can conveniently be delivered in the form of an aerosol spray using a pressurized pack or a nebulizer and a suitable propellant The compounds may also be formulated in rectal compositions such as suppositories or retention enemas, using, e.g., conventional suppository bases such as cocoa butter or other glycerides.

In addition to the formulations described previously, the compounds may also be formulated as depot preparations. Such long acting formulations may be administered by implantation (for example, subcutaneously or intramuscularly) or by intramuscular injection. The compounds of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

Additionally, the compounds may be delivered using a sustained-release system, such as semi-permeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the particular compound, additional stabilization strategies may be employed.

Other delivery systems such as liposomes and emulsions can also be used.

A therapeutically effective amount refers to an amount of compound effective to prevent, alleviate or ameliorate disease symptoms. Determination of a therapeutically effective amount is well within the capability of those skilled in the art, especially in light of the disclosure herein.

For any compound used in the methods of the invention, the therapeutically effective amount can be estimated initially from in vitro assays. Then, the dosage can be formulated for use in animal models so as to achieve a circulating concentration range that includes the effective dosage. Such information can then be used to more accurately determine dosages useful in patients.

The amount of the composition that is administered will depend upon the parent molecule included therein. Generally, the amount used in the treatment methods is that amount which effectively achieves the desired therapeutic result in mammals. Naturally, the dosages of the various compounds can vary somewhat depending upon the compound, rate of in vivo hydrolysis, etc. In addition, the dosage, of course, can vary depending upon the dosage form and route of administration.

Alternatively and preferably, the amounts of the compounds administered can be based on body surface of human or other mammals. Thus, the treatment of the present invention includes administering the compounds described herein in an amount of from about 0.1 to about 45 mg/m2 body surface/dose.

It is contemplated that the treatment will be given for one or more cycles until the desired clinical result is obtained. The exact amount, frequency and period of administration of the compound of the present invention will vary, of course, depending upon the sex, age and medical condition of the patient as well as the severity of the disease as determined by the attending clinician.

### EXAMPLES

Examples 1-64 are examples falling within the scope of the invention, as described by general formula (I).

### Examples 1-64

### According to General Formula (I)

(I)

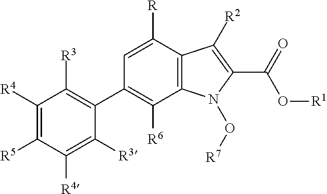

wherein

| Ex. | R | R¹ | R² | R³ | R⁴ | R⁵ | R³′ | R⁴′ | R⁶ | R⁷ | Q |
|---|---|---|---|---|---|---|---|---|---|---|---|
| 1 | CF₃ | C₂H₅ | H | H | H | H | H | H | H | H | — |
| 2 | CF₃ | H | H | H | H | H | H | H | H | 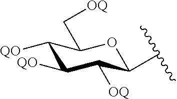 | H |
| 3 | CF₃ | CH₃ | H | H | H | H | H | H | H | 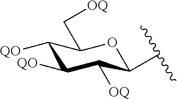 | H |
| 4 | CF₃ | H | H | H | H | H | H | H | H | 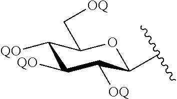 | CH₃C(O) |
| 5 | CF₃ | CH₃ | H | H | H | H | H | H | H | 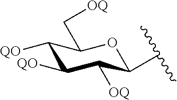 | CH₃C(O) |
| 6 | CF₃ | C₂H₅ | H | H | H | H | H | H | H | 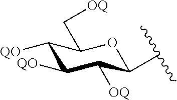 | H |
| 7 | CF₃ | C₂H₅ | H | H | H | H | H | H | H | 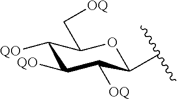 | CH₃C(O) |
| 8 | CF₃ | CH₃ | H | Cl | H | Cl | H | H | H | H | — |
| 9 | CF₃ | C₂H₅ | H | Cl | H | Cl | H | H | H | H | — |
| 10 | CF₃ | CH₃ | H | Cl | H | Cl | H | H | H | 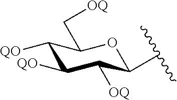 | H |
| 11 | CF₃ | CH₃ | H | Cl | H | Cl | H | H | H | 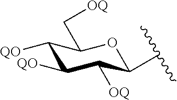 | CH₃C(O) |
| 12 | CF₃ | C₂H₅ | H | Cl | H | Cl | H | H | H | 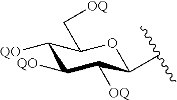 | H |
| 13 | CF₃ | C₂H₅ | H | Cl | H | Cl | H | H | H | 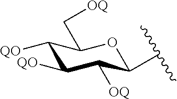 | CH₃C(O) |
| 14 | CF₃ | CH₃ | H | H | H | H | H | H | C₆H₅ | H | — |
| 15 | CF₃ | CH₃ | H | H | H | Cl | H | H | H | H | — |
| 16 | CF₃ | CH₃ | CH₃ | H | H | H | H | H | H | H | — |
| 17 | CF₃ | CH₃ | CH₃ | H | H | Cl | H | H | H | H | — |
| 18 | CF₃ | CH₃ | H | H | H | CF₃O | H | H | H | H | — |
| 19 | CF₃ | CH₃ | H | H | CF₃O | H | H | H | H | H | — |
| 20 | CF₃ | CH₃ | CH₃ | Cl | H | Cl | H | H | H | H | — |
| 21 | CF₃ | CH₃ | H | H | Cl | Cl | H | H | H | H | — |
| 22 | CF₃ | (CH₂)₃CH₃ | H | H | H | H | H | H | H | H | — |
| 23 | CF₃ | CH(CH₃)₂ | H | H | H | H | H | H | H | H | — |
| 24 | CF₃ | H | H | H | H | CF₃O | H | H | H | H | — |
| 25 | CF₃ | H | H | H | CF₃O | H | H | H | H | H | — |
| 26 | CF₃ | H | CH₃ | Cl | H | Cl | H | H | H | H | — |
| 27 | CF₃ | H | H | H | Cl | Cl | H | H | H | H | — |
| 28 | CF₃ | (CH₂)₃CH₃ | H | H | H | H | H | H | H | 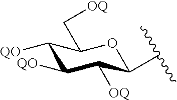 | CH₃C(O) |
| 29 | CF₃ | (CH₂)₃CH₃ | H | H | H | H | H | H | H | 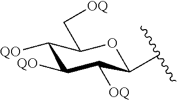 | H |
| 30 | CF₃ | CH(CH₃)₂ | H | H | H | H | H | H | H | 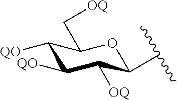 | CH₃C(O) |
| 31 | CF₃ | CH(CH₃)₂ | H | H | H | H | H | H | H | 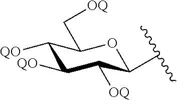 | H |
| 32 | CF₃ | CH(CH₃)₂ | H | Cl | H | Cl | H | H | H | H | — |
| 33 | CF₃ | (CH₂)₃CH₃ | H | Cl | H | Cl | H | H | H | H | — |
| 34 | CF₃ | CH₃ | H | OCF₃ | H | H | H | H | H | H | — |
| 35 | CF₃ | H | H | OCF₃ | H | H | H | H | H | H | — |
| 36 | CF₃ | CH₃ | H | Cl | Cl | H | H | H | H | H | — |
| 37 | CF₃ | H | H | Cl | Cl | H | H | H | H | H | — |
| 38 | CF₃ | CH₃ | H | Cl | H | H | H | Cl | H | H | — |
| 39 | CF₃ | H | H | Cl | H | H | H | Cl | H | H | — |
| 40 | CF₃ | CH₃ | H | H | H | H | H | H | H | 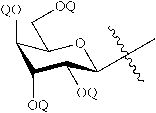 | H |
| 41 | CF₃ | CH₃ | H | H | H | H | H | H | H | 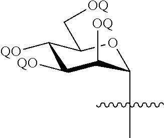 | H |
| 42 | CF₃ | CH₃ | H | H | Cl | H | H | Cl | H | H | — |
| 43 | CF₃ | H | H | H | Cl | H | H | Cl | H | H | — |
| 44 | CF₃ | CH(CH₃)₂ | H | Cl | H | Cl | H | H | H | 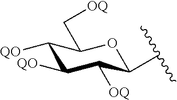 | CH₃C(O) |
| 45 | CF₃ | CH(CH₃)₂ | H | Cl | H | Cl | H | H | H | 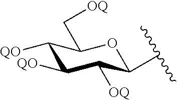 | H |
| 46 | CF₃ | (CH₂)₃CH₃ | H | Cl | H | Cl | H | H | H | 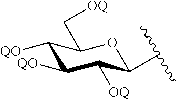 | CH₃C(O) |
| 47 | CF₃ | (CH₂)₃CH₃ | H | Cl | H | Cl | H | H | H | 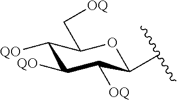 | H |
| 48 | CF₃ | CH₂(C₆H₅) | H | H | H | H | H | H | H | H | — |
| 49 | CF₃ | 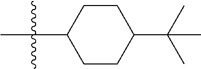 [SMILES: *C1CCC(C(C)(C)C)CC1] | H | H | H | H | H | H | H | H | — |
| 50 | CF₃ | 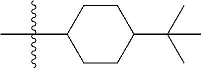 [SMILES: *C1CCC(C(C)(C)C)CC1] | H | Cl | H | Cl | H | H | H | H | — |
| 51 | F | CH₃ | H | H | H | H | H | H | H | H | — |
| 52 | F | H | H | H | H | H | H | H | H | H | — |
| 53 | F | CH₃ | H | Cl | H | Cl | H | H | H | H | — |
| 54 | F | H | H | Cl | H | Cl | H | H | H | H | — |
| 55 | CF₃ | CH₂(C₆H₅) | H | Cl | H | Cl | H | H | H | H | — |
| 56 | CF₃ | 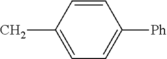 | H | Cl | H | Cl | H | H | H | H | — |
| 57 | CF₃ | 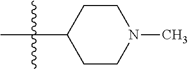 [SMILES: *C1CCN(C)CC1] | H | H | H | H | H | H | H | H | — |
| 58 | CF₃ | 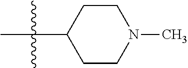 [SMILES: *C1CCN(C)CC1] | H | Cl | H | Cl | H | H | H | H | — |
| 59 | CF₃ | 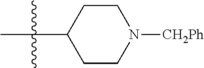 | H | Cl | H | Cl | H | H | H | H | — |
| 60 | CF₃ | 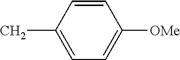 | H | Cl | H | Cl | H | H | H | H | — |
| 61 | CF₃ | 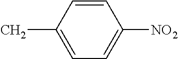 | H | Cl | H | Cl | H | H | H | H | — |
| 62 | CF₃ | 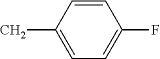 | H | Cl | H | Cl | H | H | H | H | — |
| 63 | CF₃ | 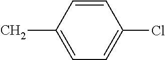 | H | Cl | H | Cl | H | H | H | H | — |
| 64 | CF₃ | 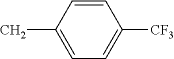 | H | Cl | H | Cl | H | H | H | H | — |

The IUPAC names of the above examples are listed below:
ethyl 1-hydroxy-6-phenyl-4-(trifluoromethyl)-1H-indole-2-carboxylate (Example 1);
6-phenyl-1-(β-D-glucopyranosyl)oxy-4-(trifluoromethyl)-1H-indole-2-carboxylic acid (Example 2);
methyl 6-phenyl-1-(β-D-glucopyranosyl)oxy-4-(trifluoromethyl)-1H-indole-2-carboxylate (Example 3);
6-phenyl-1-(β-d-2,3,4,6-tetra-O-acetylglucopyranosyl)oxy-4-(trifluoromethyl)-1H-indole-2-carboxylic acid (Example 4);
methyl 6-phenyl-1-(β-d-2,3,4,6-tetra-O-acetylglucopyranosyl)oxy-4-(trifluoromethyl)-1H-indole-2-carboxylate (Example 5);
ethyl 6-phenyl-1-(β-D-glucopyranosyl)oxy-4-(trifluoromethyl)-1H-indole-2-carboxylate (Example 6);
ethyl 6-phenyl-1-(β-d-2,3,4,6-tetra-O-acetylglucopyranosyl)oxy-4-(trifluoromethyl)-1H-indole-2-carboxylate (Example 7);
methyl 6-(2,4-dichlorophenyl)-1-hydroxy-4-(trifluoromethyl)-1H-indole-2-carboxylate (Example 8);
ethyl 6-(2,4-dichloropheyl)-1-hydroxy-4-(trifluoromethyl)-1H-indole-2-carboxylate (Example 9);
methyl 6-(2,4-dichlorophenyl)-1-(β-D-glucopyranosyl)oxy-4-(trifluoromethyl)-1H-indole-2-carboxylate (Example 10);
methyl 6-(2,4-dichlorophenyl)-1-(β-d-2,3,4,6-tetra-O-acetylglucopyranosyl)oxy-4-(trifluoromethyl)-1H-indole-2-carboxylate (Example 11);
ethyl 6-(2,4-dichlorophenyl)-1-(β-D-glucopyranosyl)oxy-4-(trifluoromethyl)-1H-indole-2-carboxylate (Example 12);
ethyl 6-(2,4-dichlorophenyl)-1-(β-d-2,3,4,6-tetra-O-acetylglucopyranosyl)oxy-4-(trifluoromethyl)-1H-indole-2-carboxylate (Example 13);
methyl 1-hydroxy-6,7-diphenyl-4-(trifluoromethyl)-1H-indole-2-carboxylate (Example 14);
methyl 6-(4-clorophenyl)-1-hydroxy-4-(trifluoromethyl)-1H-indole-2-carboxylate (Example 15);
methyl 1-hydroxy-6-phenyl-3-methyl-4-(trifluoromethyl)-1H-indole-2-carboxylate (Example 16);
methyl 1-hydroxy-6-(4-clorophenyl)-3-methyl-4-(trifluoromethyl)-1H-indole-2-carboxylate (Example 17);
methyl 1-hydroxy-4-(trifluoromethyl)-6-(4-(trifluoromethoxy)phenyl)-1H-indole-2-carboxylate (Example 18);
methyl 1-hydroxy-4-(trifluoromethyl)-6-(3-(trifluoromethoxy)phenyl)-1H-indole-2-carboxylate (Example 19);
methyl 6-(2,4-dichlorophenyl)-1-hydroxy-3-methyl-4-(trifluoromethyl)-1H-indole-2-carboxylate (Example 20);
methyl 6-(3,4-dichlorophenyl)-1-hydroxy-4-(trifluoromethyl)-1H-indole-2-carboxylate (Example 21);
butyl 1-hydroxy-6-phenyl-4-(trifluoromethyl)-1H-indole-2-carboxylate (Example 22);
isopropyl 1-hydroxy-6-phenyl-4-(trifluoromethyl)-1H-indole-2-carboxylate (Example 23);
1-hydroxy-4-(trifluoromethyl)-6-(4-(trifluoromethoxy)phenyl)-1H-indole-2-carboxylic acid (Example 24);
1-hydroxy-4-(trifluoromethyl)-6-(3-(trifluoromethoxy)phenyl)-1H-indole-2-carboxylic acid (Example 25);
6-(2,4-dichlorophenyl)-1-hydroxy-3-methyl-4-(trifluoromethyl)-1H-indole-2-carboxylic acid (Example 26);
6-(3,4-dichlorophenyl)-1-hydroxy-4-(trifluoromethyl)-1H-indole-2-carboxylic acid (Example 27);
butyl 6-phenyl-1-(β-D-2,3,4,6-tetra-O-acetylglucopyranosyl)oxy-4-(trifluoromethyl)-1H-indole-2-carboxylate (Example 28);
butyl 1-(β-D-glucopyranosyl)oxy-6-phenyl-4-(trifluoromethyl)-1H-indole-2-carboxylate (Example 29);
isopropyl 6-phenyl-1-(β-D-2,3,4,6-tetra-O-acetylglucopyranosyl)oxy-4-(trifluoromethyl)-1H-indole-2-carboxylate (Example 30);
isopropyl 1-(β-D-glucopyranosyl)oxy-6-phenyl-4-(trifluoromethyl)-1H-indole-2-carboxylate (Example 31);
isopropyl 6-(2,4-dichlorophenyl)-1-hydroxy-4-(trifluoromethyl)-1H-indole-2-carboxylate (Example 32);
butyl 6-(2,4-dichlorophenyl)-1-hydroxy-4-(trifluoromethyl)-1H-indole-2-carboxylate (Example 33);
methyl 1-hydroxy-6-(2-(trifluoromethoxy)phenyl)-4-(trifluoromethyl)-1H-indole-2-carboxylate (Example 34);
1-hydroxy-6-(2-(trifluoromethoxy)phenyl)-4-(trifluoromethyl)-1H-indole-2-carboxylic acid (Example 35);
methyl 6-(2,3-dichlorophenyl)-1-hydroxy-4-(trifluoromethyl)-1H-indole-2-carboxylate (Example 36);
6-(2,3-dichlorophenyl)-1-hydroxy-4-(trifluoromethyl)-1H-indole-2-carboxylic acid (Example 37);
methyl 6-(2,5-dichlorophenyl)-1-hydroxy-4-(trifluoromethyl)-1H-indole-2-carboxylate (Example 38);
6-(2,5-dichlorophenyl)-1-hydroxy-4-(trifluoromethyl)-1H-indole-2-carboxylic acid (Example 39);
methyl 1-(β-d-gulopyranosyl)oxy-6-phenyl-4-(trifluoromethyl)-1H-indole-2-carboxylate (Example 40);
methyl 1-(α-d-mannopyranosyl)oxy-6-phenyl-4-(trifluoromethyl)-1H-indole-2-carboxylate (Example 41);
methyl 6-(3,5-dichlorophenyl)-1-hydroxy-4-(trifluoromethyl)-1H-indole-2-carboxylate (Example 42);
6-(3,5-dichlorophenyl)-1-hydroxy-4-(trifluoromethyl)-1H-indole-2-carboxylic acid (Example 43);
isopropyl 6-(2,4-dichlorophenyl)-1-(β-d-2,3,4,6-tetra-O-acetylglucopyranosyl)oxy-4-(trifluoromethyl)-1H-indole-2-carboxylate (Example 44);
isopropyl 6-(2,4-dichlorophenyl)-1-(β-D-glucopyranosyl)oxy-4-(trifluoromethyl)-1H-indole-2-carboxylate (Example 45);
butyl 6-(2,4-dichlorophenyl)-1-(β-d-2,3,4,6-tetra-O-acetylglucopyranosyl)oxy-4-(trifluoromethyl)-1H-indole-2-carboxylate (Example 46);
butyl 6-(2,4-dichlorophenyl)-1-(β-D-glucopyranosyl)oxy-4-(trifluoromethyl)-1H-indole-2-carboxylate (Example 47);
benzyl 1-hydroxy-6-phenyl-4-(trifluoromethyl)-1H-indole-2-carboxylate (Example 48);
4-(tert-butyl)cyclohexyl 1-hydroxy-6-phenyl-4-(trifluoromethyl)-1H-indole-2-carboxylate (Example 49);
4-(tert-butyl)cyclohexyl 6-(2,4-dichlorophenyl)-1-hydroxy-4-(trifluoromethyl)-1H-indole-2-carboxylate (Example 50);
methyl 4-fluoro-1-hydroxy-6-phenyl-1H-indole-2-carboxylate (Example 51);
4-fluoro-1-hydroxy-6-phenyl-1H-indole-2-carboxylic acid (Example 52);
methyl 6-(2,4-dichlorophenyl)-4-fluoro-1-hydroxy-1H-indole-2-carboxylate (Example 53);
6-(2,4-dichlorophenyl)-4-fluoro-1-hydroxy-1H-indole-2-carboxylic acid (Example 54);
benzyl 6-(2,4-dichlorophenyl)-1-hydroxy-4-(trifluoromethyl)-1H-indole-2-carboxylate (Example 55);
[1,1′-biphenyl]-4-ylmethyl 6-(2,4-dichlorophenyl)-1-hydroxy-4-(trifluoromethyl)-1H-indole-2-carboxylate (Example 56);
1-methylpiperidin-4-yl 1-hydroxy-6-phenyl-4-(trifluoromethyl)-1H-indole-2-carboxylate (Example 57);
1-methylpiperidin-4-yl 6-(2,4-dichlorophenyl)-1-hydroxy-4-(trifluoromethyl)-1H-indole-2-carboxylate (Example 58);
1-benzylpiperidin-4-yl 6-(2,4-dichlorophenyl)-1-hydroxy-4-(trifluoromethyl)-1H-indole-2-carboxylate (Example 59);
4-methoxybenzyl 6-(2,4-dichlorophenyl)-1-hydroxy-4-(trifluoromethyl)-1H-indole-2-carboxylate (Example 60);
4-nitrobenzyl 6-(2,4-dichlorophenyl)-1-hydroxy-4-(trifluoromethyl)-1H-indole-2-carboxylate (Example 61);
4-fluorobenzyl 6-(2,4-dichlorophenyl)-1-hydroxy-4-(trifluoromethyl)-1H-indole-2-carboxylate (Example 62);
4-chlorobenzyl 6-(2,4-dichlorophenyl)-1-hydroxy-4-(trifluoromethyl)-1H-indole-2-carboxylate (Example 63);
4-(trifluoromethyl)benzyl 6-(2,4-dichlorophenyl)-1-hydroxy-4-(trifluoromethyl)-1H-indole-2-carboxylate (Example 64).

The compounds of the present invention can be prepared according to the procedures described in the following schemes, specific for each series of examples.

However, a person skilled in the art would understand that known variations of conditions and processes according to the following procedures can be used to prepare these compounds.

In the procedures described below all temperatures are in Celsius degrees.

The following abbreviations, reagents, expressions or machines, used in the following description, are explained as follows:

20-25° C. (room temperature), Molar equivalents (eq.), tetrabutylammonium bromide (TBAB), microwave (MW), aqueous solution (aq.), 1,2-dimethoxyethane (DME), dichloromethane (DCM), chloroform (CHCl₃), ethyl acetate (EtOAc), tetrahydrofuran (THF), methanol (MeOH), diethyl ether (Et₂O), dimethylsulfoxide (DMSO), sodium hydride (NaH), dimethyl oxalate (“(COOMe)₂”), stannous chloride (SnCl₂), ammonium chloride (NH₄Cl), metallic zinc powder (Zn), triethylsilane (Et₃SiH), lithium hydroxide (LiOH), hydrochloric acid (HCl), acetic acid (AcOH), sodium bicarbonate (NaHCO₃), normal concentration (N), molar concentration (M), dimethyl formamide (DMF), carbonyldiimidazole (CDI), millimoles (mmol), milliliter (mL), microliters (mL), nanometers (nm), Ångström (Å), chromatography on thin layer (TLC), nuclear magnetic resonance (MMR), electron impact mass spectrometry (EI/MS), mass spectrometry coupled with gas chromatography (GC//MS), Eagle modified Dulbecco's medium or “Dulbecco's Modified Eagle's Medium” (DMEM), fetal bovine serum (FBS), solution of 5000 units/mL of sodium salt of penicillin G and 5000 micrograms/mL of base streptomycin in saline solution at 0.85% (Pen-strep), tert-butyldimethylclorosylane (TBDMCS), N-methyl-N-(tert-butyldimethylsilyl)trifluoroacetamide (MTBSTFA), p-clorophenylalanine (CPA), gas chromatography (GC), sulforhodamine B (SRB).

The examples 1-64 were prepared as shown in the general procedure of schemes 1-5.

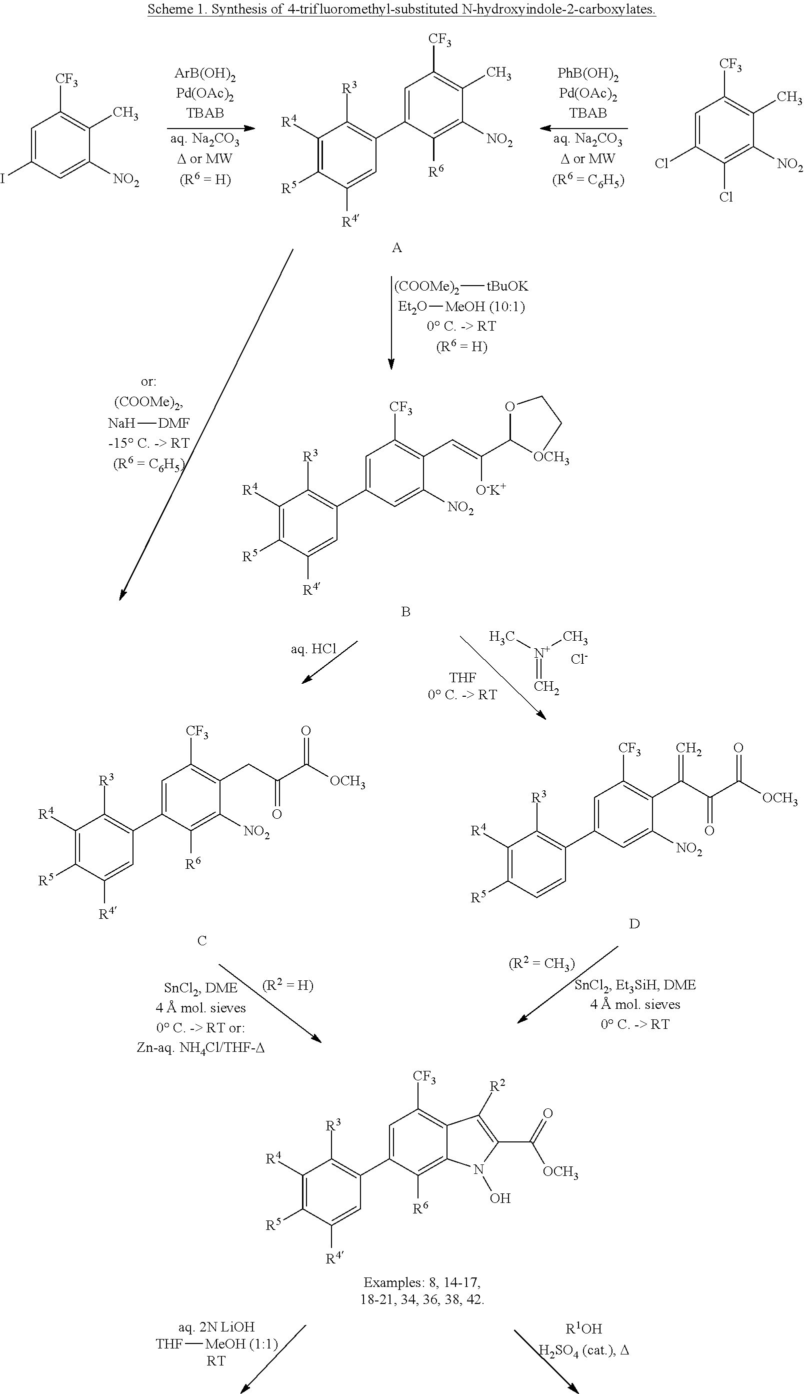

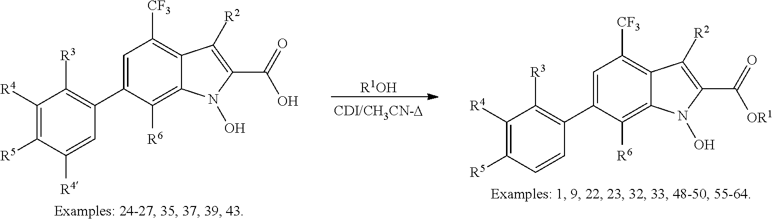

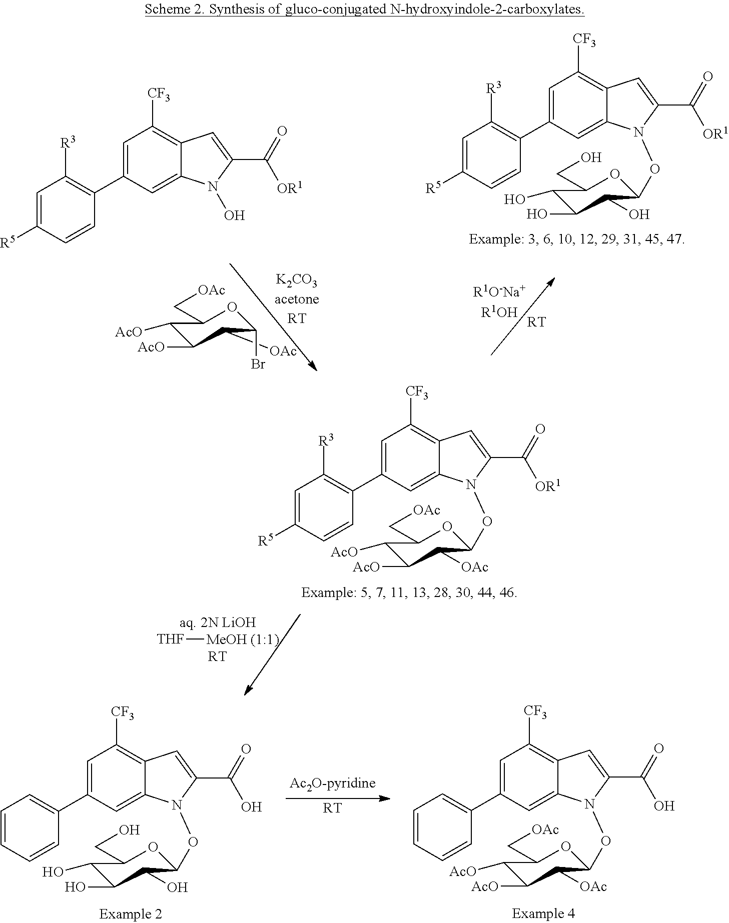

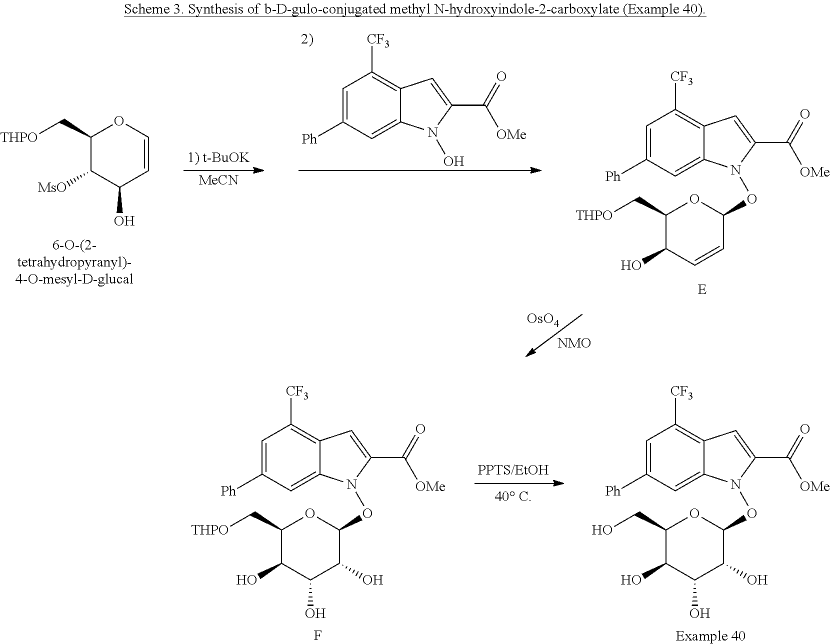

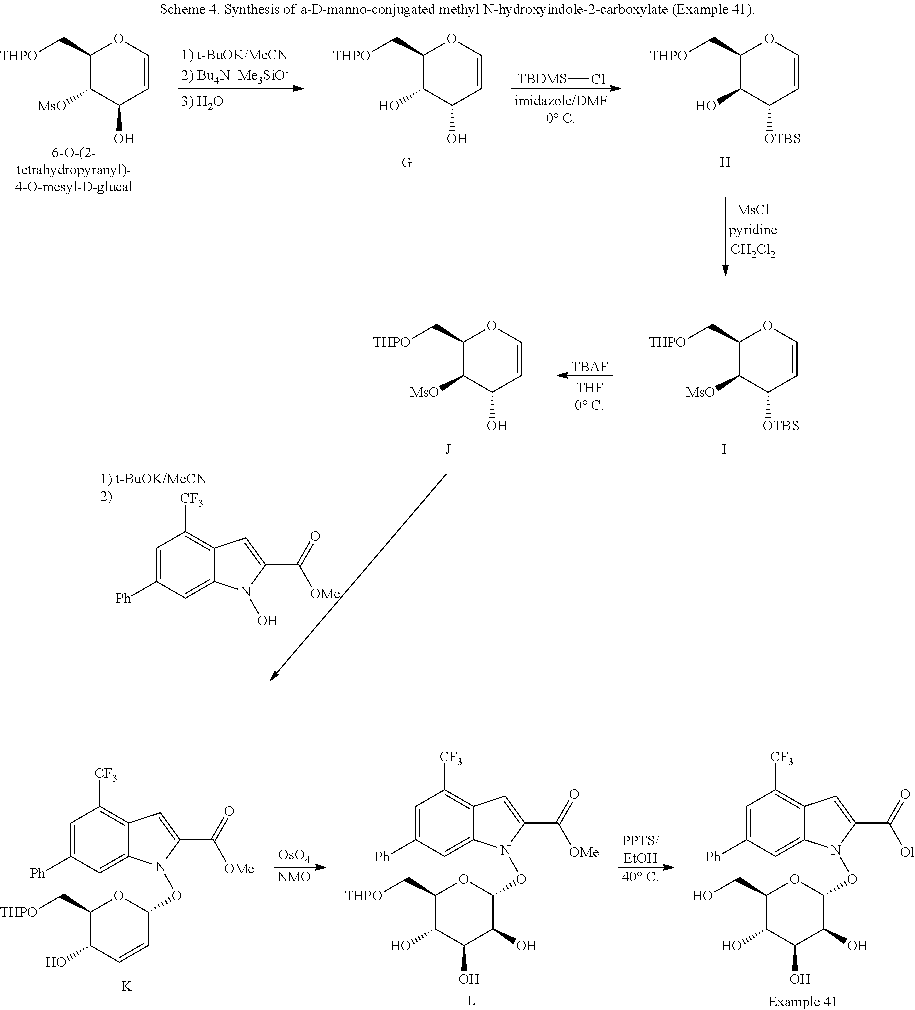

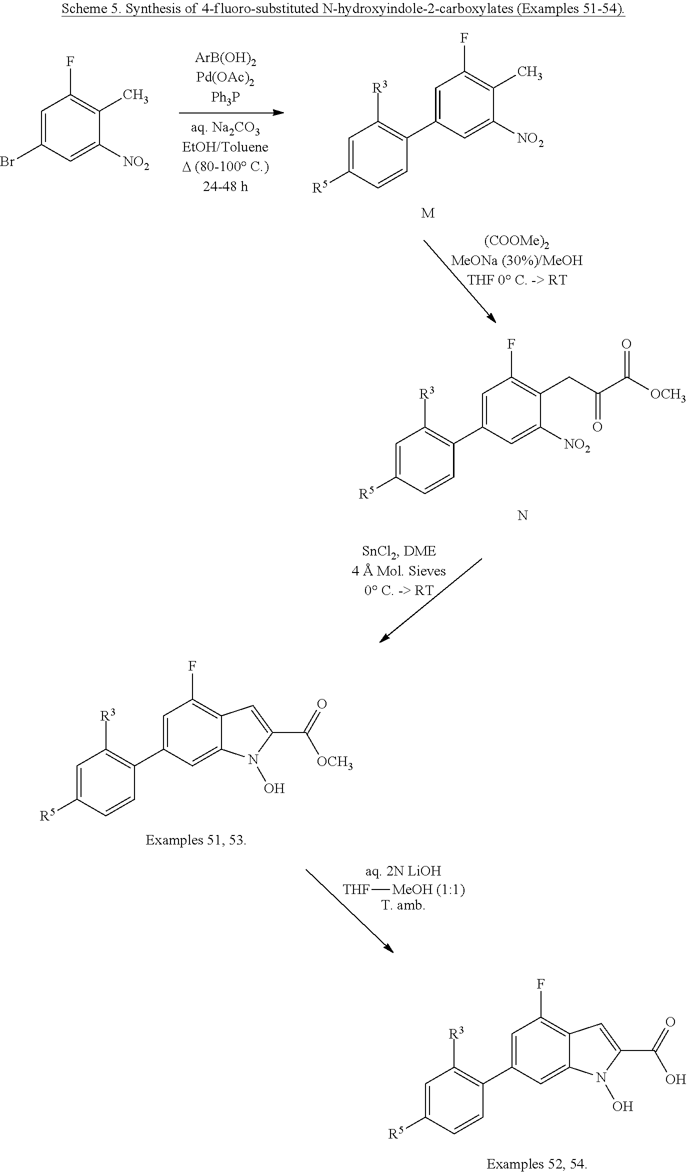

### Procedures for the Preparation of Representative Examples

(for characterization data of the final products, see the next section “Characterization data of all the examples”).

### Example 5

Methyl 1-hydroxy-6-phenyl-4-(trifluoromethyl)-1H-indole-2-carboxylate [Granchi, C., et al. J. Med. Chem. 2011, 54, 1599-1612] (386 mg, 1.15 mmol) was added to a suspension of anhydrous potassium carbonate (952 mg, 6.90 mmol) in anhydrous acetone (8 mL) under inert atmosphere and the resulting suspension was treated with 2,3,4,6-tetra-O-acetyl-α-D-glucopyranosyl bromide (945 mg, 2.30 mmol). After 24 hours of stirring and protected from light, the solvent was removed under vacuum and the residue was extracted with EtOAc. The resulting organic phase was washed with brine and dried over anhydrous sodium sulphate and after evaporation under vacuum gave a solid residue, which was recrystallized from a mixture of n-hexane and EtOAc, to give 598 mg (0.900 mmol, yield 78%) of white crystals of methyl 6-phenyl-1-(β-D-2,3,4,6-tetra-O-acetylglucopyranosyl)oxy-4 (trifluoromethyl)-1H-indole-2-carboxylate (Example 5).

### Example 3

The compound 5 (400 mg, 0.602 mmol) was dissolved under inert atmosphere in anhydrous methanol (25 mL) and, after mild heating to speed up the dissolution, the mixture was cooled to 0° C. and treated at the same temperature with a solution of 30% sodium methoxide in methanol (0.25 mL). The resulting solution was stirred for about 3 hours at room temperature, or at least until the disappearance of the starting compound had been verified by TLC analysis. The reaction mixture was then treated with acidic resin Amberlitem IR 120 H, until reaching a neutral pH value. The resulting suspension was filtered to remove the resin, which was further rinsed with MeOH, and the filtrate was concentrated under vacuum to obtain (293 mg, 0.590 mmol, yield of 98%) methyl 6-phenyl-1-(β-D-glucopyranosyl)oxy-4-(trifluoromethyl)-1H-indole-2-carboxylate as a white solid (Example 3).

### Example 2

The compound 3 (62 mg, 0.12 mmol) was dissolved under inert atmosphere in a mixture 1:1 (volume/volume) of THF/MeOH. A previously degassed 2N aqueous solution of lithium hydroxide (0.4 mL) was added dropwise to the resulting solution under constant flow of nitrogen. After having verified by TLC analysis the disappearance of the starting compound, the reaction mixture was treated with acidic resin Amberlite™ IR 120 H, until reaching a pH value of about 2. The resulting suspension was filtered to remove the resin, which was further rinsed with MeOH, and the filtrate was concentrated under vacuum to obtain 54 mg (0.11 mmol, 92% yield) of 6-phenyl-1-(β-D-glucopyranosyl)oxy-4-(trifluoromethyl)-1H-indole-2-carboxylic acid (Example 2) as a white solid.

### Example 4

Compound 2 (50 mg, 0.10 mmol) was dissolved under inert atmosphere in pyridine (0.8 mL) and acetic anhydride (0.4 mL) was added dropwise. The reaction mixture was stirred at room temperature protected from light for 24 hours. The mixture was then subjected to cycles of co-evaporation under vacuum with toluene, to remove the pyridine and acetic anhydride. The residue was placed on a preparative TLC plate and eluted with a 95:5 mixture of DCM/MeOH providing (25 mg, 0.038 mmol, yield 37%) 6-phenyl-1-(β-D-2,3,4,6-tetra-O-acetilglucopiranosil)oxy-4-(trifluoromethyl)-1H-indole-2-carboxylic acid as a white solid (Example 4).

### Example 9

In the first step [Similar procedures have been previously described in: (a) Miyaura, N.; Suzuki, A. Chem. Rev. 1995, 95, 2457-2483; (b) Suzuki, A. J. Organomet. Chem. 1999, 576, 147-168, and references therein], a solution containing palladium (II) acetate (10 mg, 0.045 mmol) and triphenylphosphine (59.0 mg, 0.225 mmol) in absolute ethanol (3.5 mL) and anhydrous toluene (3.5 mL) was stirred under inert atmosphere at room temperature for minutes. Subsequently, 5-iodo-2-methyl-1-nitro-3-(trifluoromethyl) benzene (497 mg, 1.50 mmol), 3.5 mL of a 2M aqueous solution of sodium carbonate, and 2,4-dichlorophenylboronic acid (720 mg, 3.77 mmol) were added. The resulting mixture was heated to 100° C. in a sealed vial under inert atmosphere for 24 hours, or in any case after having verified by TLC analysis the disappearance of the starting compound in stoichiometric defect (the iodoaryle). After cooling to room temperature, the mixture was diluted with water and extracted several times with EtOAc. The combined organic phases were dried over anhydrous sodium sulfate and concentrated under vacuum.

The crude residue was purified by flash column chromatography with n-hexane as eluent to give 475 mg (1.36 mmol, 90% yield) of the corresponding intermediate “A” [Scheme 1, R³ and R⁵═Cl; R⁴ and R⁶═H; ¹H NMR (CDCl₃): δ (ppm) 2.62 (q, 3H, J=1.5 Hz), 7.29 (d, 1H, J=8.2 Hz), 7.38 (dd, 1H, J=8.2, 2.0 Hz), 7.55 (d, 1H, J=2.0 Hz), 7.92 (d, 1H, J=1.8 Hz), 7.98 (d, 1H, J=1.8 Hz)].

In the next step [similar procedure previously described in Dong, W., Jimenez, L. S. J. Org. Chem. 1999, 64, 2520-2523] is suspended in potassium tert-butoxide (469 mg, 4.18 mmol) in anhydrous Et₂O (7 mL) at 0° C. under inert atmosphere. Anhydrous methanol (about 0.5 mL) was added until reaching complete dissolution. Then, dimethyl oxalate (494 mg, 4.18 mmol) was added and the mixture was stirred at 0° C. for further 15 minutes. Finally, a solution containing the intermediate “A” of the previous step (1.22 g, 3.48 mmol) in anhydrous Et₂O (4.5 mL) was slowly added maintaining at 0° C. The resulting reddish suspension was then stirred for additional 24 hours giving the potassium enolate “B” [Scheme 1, R³ and R⁵═Cl; R⁴ and R⁶=1-1] that almost completely precipitated from the reaction medium. This intermediate was used in the subsequent step without any purification.

The reaction mixture containing intermediate “B” was diluted with EtOAc and an aqueous solution of 1N HCl. The organic phase was separated, washed with brine, dried over anhydrous sodium sulphate and concentrated under vacuum. The crude reaction product was purified by flash column chromatography (eluent: mixture of 9:1 n-hexane/EtOAc), to give 687 mg (1.58 mmol, yield 45%) of the corresponding intermediate “C” [Scheme 1, R³ and R⁵═Cl; R⁴ and R⁶═H; ¹H NMR (CDCl₃): δ (ppm) 3.98 (s, 3H), 4.74 (s, 2H), 7.33 (d, 1H, J=8.4 Hz), 7.41 (dd, 1H, J=8.2, 2.0 Hz), 7.57 (d, 1H, J=1.8 Hz), 8.06 (d, 1H, J=1.6 Hz), 8.33 (d, 1H, J=1.8 Hz)].

In the last step [similar procedure described in: Nicolaou, K. C., Estrada, A. A., Freestone, G. C., Lee, S. H., Alvarez-Mico, X. Tetrahedron 2007, 63, 6088-6114], intermediate “C” obtained as described above (680 mg, 1.56 mmol) was dissolved in anhydrous DME (1.5 mL) and the resulting solution was added dropwise to a solution containing stannous chloride (662 mg, 3.49 mmol) in anhydrous DME (1.5 mL) cooled down to 0° C. and in the presence of molecular sieves 4 Å, which were previously activated in an oven at 130° C. for 18 hours and cooled in a desiccator containing anhydrous calcium chloride. The resulting mixture was stirred under inert atmosphere at room temperature for 20 hours, or at least until the almost complete disappearance of the starting compound by TLC analysis had been verified. Then, the mixture was diluted with water and extracted with EtOAc. The combined organic phases were dried over anhydrous sodium sulfate and concentrated under vacuum to provide a crude residue, which was purified by flash column chromatography (eluent: mixture of 8:2 n-hexane/EtOAc) to give 431 mg (1.07 mmol, yield of 68%) of methyl 6-(2,4-dichlorophenyl)-1-hydroxy-4-(trifluoromethyl)-1H-indole-2-carboxylate (Example 8). This compound (330 mg, 0.816 mmol) was dissolved in absolute ethanol (20 mL) containing a small amount (7 drops) of concentrated sulfuric acid. The resulting mixture was heated to reflux in a flask for 48 hours, or at least until the disappearance of the starting compound by TLC analysis had been verified. Most of the solvent was then removed under vacuum, and the residue taken up with EtOAc. The organic phase thus obtained was washed with brine, dried over anhydrous sodium sulfate and concentrated under vacuum. The crude residue was then purified by flash column chromatography, with a 85:15 mixture of n-hexane/EtOAc as eluent, to give 295 mg (0.705 mmol, yield 86%) of ethyl 6-(2,4-dichlorophenyl)-1-hydroxy-4-(trifluoromethyl)-1H-indole-2-carboxylate (Example 9).

### Example 14

In the first step [similar procedure previously described in: Leadbeater, N. E., Marco, M. Org. Lett 2002, 4, 2973-2976] the commercial derivative 3,4-dichloro-2-nitro-6-(trifluoromethyl) toluene (274 mg, 1.00 mmol) was placed in a sealed vial under an inert atmosphere in a microwave reactor together with phenylboronic acid (366 mg, 3.00 mmol), sodium carbonate (636 mg, 6.00 mmol), palladium (II) acetate (2.5 mg, 0.01 mmol), tetrabutylammonium bromide (660 mg, 2.00 mmol) and water (3.0 mL). The mixture was subjected to microwave irradiation under stirring at 175° C. for 10 minutes. After dilution with water and repeated extraction with EtOAc, the combined organic phases were dried over anhydrous sodium sulfate and concentrated under vacuum to give a crude residue, which was then purified by flash column chromatography with a 95:5 mixture of n-hexane/EtOAc as eluent, to give 296 mg (0.828 mmol, 83% yield) of the corresponding intermediate “A” [Scheme 1, R³-R⁵═H; R⁶═C₆H₅; ¹H NMR (CDCl₃): δ (ppm) 2.47 (q, 3H, J=1.5 Hz), 7:01 to 7:11 (m, 5H), 7:16 to 7:28 (m, 5H), 7.82 (s, 1H)]. In the next step [similar procedure previously described in: Nicolaou, K. C., Estrada, A. A., Freestone, G. C., Lee, S. H., Alvarez-Mico, X. Tetrahedron 2007, 63, 6088-6114], a solution containing the intermediate “A” (290 mg, 0.812 mmol) and dimethyl oxalate (479 mg, 4.06 mmol) obtained as described above in 5 mL of anhydrous DMF was dropwise added to an oily suspension of 60% sodium hydride (130 mg, 3.25 mmol) in 5 mL of anhydrous DMF at −15° C. under nitrogen. After the addition, the mixture was kept for 10 minutes at the same temperature and then allowed to slowly reach room temperature. After a certain period of time, which varied depending on the substrate, the development of intense colors ranging from cherry red to bluish-purple was observed. The mixture was left under stirring at room temperature for 18 hours. Once verified the disappearance of the nitroarylic precursor by TLC, the reaction mixture was poured into ice and water; the aqueous phase was acidified with 1N HCl and extracted several times with EtOAc. The combined organic phases were washed with a 6% NaHCO₃ solution, brine and then dried over anhydrous sodium sulfate. The evaporation of the organic solvent gave a crude residue, which was then purified by flash column chromatography using a mixture of 8:2 n-hexane/EtOAc as eluent, to give 342 mg (0.771 mmol, 95% yield) of the corresponding intermediate “C” [Scheme 1, R³-R⁵═H; R⁶═C₆H₅; ¹H NMR (CDCl₃): δ (ppm) 3.95 (s, 3H), 4.42 (s, 2H), 7:05 to 7:09 (m, 5H), 7:21 to 7:25 (m, 5H), 7.91 (s, 1H)]. In the next step [similar procedure previously described for analogous compounds in: Nicolaou, K. C., Estrada, A. A., Freestone, G. C., Lee, S. H., Alvarez-Mico, X. Tetrahedron 2007, 63, 6088-6114], a suspension of zinc powder (82.0 mg, 1.25 mmol) and molecular iodine (16.0 mg, 0.0625 mmol) in anhydrous THF (1 mL) was vigorously stirred under an inert atmosphere and under reflux for about 3 hours. After cooling to room temperature, under an inert atmosphere, 2.0 mL of a 1N aqueous ammonium chloride solution and a solution containing the intermediate “C” (111 mg, 0.270 mmol) obtained as described above in THF (1 mL) were added. The resulting suspension was stirred at 40° C. for 2 hours, or at least until the disappearance of the starting compound by TLC analysis had been verified. The reaction mixture was repeatedly extracted with EtOAc and the combined organic phases were washed with brine, dried over anhydrous sodium sulfate and concentrated. The residue obtained was treated with glacial acetic acid and concentrated under vacuum. The resulting crude mixture was purified by flash column chromatography and a mixture of 8:2 n-hexane/EtOAc as eluent, to give 36.6 mg (0.0891 mmol, yield 33%) of methyl 1-hydroxy-6,7-diphenyl-4-(trifluoromethyl)-1H-indole-2-carboxylate (Example 14).

### Example 20

Intermediate enolate “B”, obtained as described in the procedure for Example 9 [Scheme 1, R³ and R⁵═Cl; R⁴ and R⁶═H], an collected as reddish salt by filtration of the reaction mixture (160 mg, 0.337 mmol) was directly used in the next step without further purification or characterization, using immediate dissolution in anhydrous THF (15 mL). The solution thus obtained was cooled under inert atmosphere to 0° C. and treated with dimethylmethylenammonium chloride (95 mg, 1.01 mmol) [similar procedure previously described for analogous compounds in: Nicolaou, K. C., Estrada, A. A., Freestone, G. C., Lee, S. H., Alvarez-Mico, X. Tetrahedron 2007, 63, 6088-6114]. The mixture was stirred at room temperature for further 18 hours, then treated with a saturated aqueous solution of ammonium chloride and repeatedly extracted with EtOAc. The combined organic phases are washed with water, dried over anhydrous sodium sulfate and concentrated under vacuum to give a crude residue, which was then purified by flash column chromatography, eluting with a mixture of 8:2 n-hexane/EtOAc, to give 55 mg (0.12 mmol, yield 36%) of corresponding intermediate “D” [Scheme 1, R³ and R⁵═Cl; R¹═H; ¹H NMR (CDCl₃): 5 (ppm) 3.94 (s, 3H), 6.22 (s, 1H), 6.85 (s, 1H), 7.35 (d, 1H, J=8.2 Hz), 7.42 (dd, 1H, J=8.2, 2.0 Hz), 7.59 (d, 1H, J=1.9 Hz), 8:09 (d, 1H, J=1.8 Hz), 8.35 (d, 1H, J=1.8 Hz)].

In the next step [similar procedure previously described in Dong, W., Jimenez, L. S. J. Org. Chem. 1999, 64, 2520-2523] triethylsilane (0.1 mL, 0.6 mmol) and intermediate “D” obtained as described above (54 mg, 0.12 mmol) were added under an inert atmosphere at room temperature to a solution containing stannous chloride dihydrate (68 mg, 0.30 mmol) in anhydrous DME (1 mL) in the presence of molecular sieves 4 Å, previously activated in an oven at 130° C. for 18 hours and cooled in a desiccator containing anhydrous calcium chloride. The resulting mixture was slightly heated (at 40° C.) for 3 hours and, subsequently, was diluted with water and repeatedly extracted with EtOAc. The combined organic phases are washed with brine, dried over anhydrous sodium sulfate and concentrated under vacuum to give a crude residue, which was then placed on a preparative TLC sheet and eluted with a 9:1 mixture of n-hexane/EtOAc providing methyl 6-(2,4-dichlorophenyl)-1-hydroxy-3-methyl-4-(trifluoromethyl)-1H-indole-2-carboxylate (Example 20) as a white solid (19 mg, 0.045 mmol, 38%).

### Example 26

15 mg (0.036 mmol) of methyl 6-(2,4-dichlorophenyl)-1-hydroxy-3-methyl-4-(trifluoromethyl)-1H-indole-2 carboxylate described in Example 20 and obtained as described in in the previous section, was dissolved in a 1:1 mixture of THF and methanol (1 mL) and was treated with 0.2 mL of an 2N aqueous solution of lithium hydroxide. The reaction was stirred at room temperature and protected from light for 4 hours, or at least until the disappearance of the starting compound by TLC analysis had been verified. The mixture was concentrated under vacuum to remove the organic solvents. The residual aqueous alkaline was washed with Et₂O and then acidified with a 1N HCl solution. The resulting acidic aqueous mixture was extracted with EtOAc, the combined organic phases were dried over anhydrous sodium sulfate and concentrated under vacuum to give (14 mg, 0.035 mmol, 97% yield) of 6-(2,4-dichlorophenyl)-1-hydroxy-3-methyl-4-(trifluoromethyl)-1H-indole-2-carboxylic acid (Example 26) as a white solid.

### Example 40

A solution of 6-O-(2-Tetrahydropyranyl)-4-O-mesyl-D-glucal [V. Di Bussolo, L. Checchia, M. R. Romano, M. Pineschi, P. Crotti Org. Lett. 2008, 10, 2493-2496] (0.157 g, 0.488 mmol) in CH₃CN (13 mL) was treated with t-BuOK (0.060 g, 0.54 mmol, 1.1 equiv.) and the mixture was stirred at room temperature for 30 minutes. Then, methyl 1-hydroxy-6-phenyl-4-(trifluoromethyl)-1H-indole-2-carboxylate (0.18 g, 0.54 mmol, 1.1 equiv.) was added and stirring was continued for 1 h at the same temperature. The mixture was diluted with CH₂Cl₂ and the organic phase was washed with brine and concentrated under vacuum. The crude residue was purified by flash column chromatography (1:1 n-Hexane/EtOAc, 0.01% Et₃N) to afford intermediate “E” (0.204 g, 76% yield) as a white solid. A solution of intermediate “E” (0.050 g, 0.091 mmol) in 0.4 mL of a 1:1 t-BuOH/acetone mixture was cooled to 0° C. and treated with 0.1 mL of a 50% w/v solution of N-methylmorpholin-N-oxide (NMO) in water and 0.1 mL of a 2.5% w/v solution of OsO₄ in t-BuOH. The resulting mixture was stirred at 0° C. for 2.5 h, then it was diluted with EtOAc and filtered through a 1 cm Celite® pad. Evaporation under vacuum of the filtrate gave a crude residue, which was purified by flash column chromatography (2:8 n-Hexane/EtOAc, 0.01% Et₃N) to afford intermediate “F” (0.037 g, 70% yield) as a white solid. Final deprotection step to remove the 2-tetrahydropyranyl (THP) protecting group was achieved by treating a solution of intermediate “F” (0.048 g, 0.083 mmol) in absolute EtOH (0.5 mL) with pyridinium p-toluenesulfonate (PPTS) (0.002 g, 0.008 mmol, 0.1 equiv.) at 40° C. The resulting mixture was stirred at the same temperature for 48 h, then it was diluted with Et₂O. The organic phase was washed with a saturated aqueous solution of NaHCO₃ and with brine. After evaporation under vacuum of the organic phase, the resulting crude residue was recrystallized from n-Hexane/Et₂O, to afford 0.023 g (64% yield) methyl 1-(β-D-gulopyranosyl)oxy-6-phenyl-4-(trifluoromethyl)-1H-indole-2-carboxylate (Example 40) as a white solid.

### Example 41

A solution of 6-O-(2-Tetrahydropyranyl)-4-O-mesyl-D-glucal [V. Di Bussolo, L. Checchia, M. R. Romano, M. Pineschi, P. Crotti Org. Lett. 2008, 10, 2493-2496] (0.737 g, 2.28 mmol) in anhydrous THF (11 mL) was treated with t-BuOK (0.282 g, 2.51 mmol). The resulting mixture was stirred at room temperature for 15 minutes and then treated dropwise with a freshly prepared solution of tetrabutylammonium trimethylsilanolate* (Bu₄N⁺Me₃SiO⁻, 4 equiv.) in anhydrous THF. [*Preparation of the Bu₄N⁺Me₃SiO⁻solution: a solution of Bu₄NBr (2.17 g, 6.73 mmol, 4 equiv.) in anhydrous THF (21 mL) was treated with Me₃SiOK (0.863 g, 6.73 mmol, 4 equiv.) and the reaction mixture was stirred at room temperature for 10 min, then it was diluted with THF and filtered through a 1 cm Celite® pad; the filtrate was then concentrated under vacuum to a final volume of about 10 mL.]. Stirring was continued at the same temperature for 4 h, then the mixture was diluted with Et₂O. The organic phase was washed with brine and concentrated to give a residue that was purified by flash column chromatography (3:7 n-Hexane/EtOAc, 0.01% Et₃N) to afford intermediate “G” (0.235 g, 45% yield) as an oil. A solution of intermediate “G” 0.216 g, 0.939 mmol) in anhydrous DMF (2.5 mL) at 0° C. was treated first with imidazole (0.128 g, 1.88 mmol, 2 equiv.), and then with t-butyldimethylsilyl chloride (TBDMS-Cl, 0.170 g, 1.127 mmol, 1.2 equiv.). The reaction mixture was allowed to slowly reach room temperature and stirring was continued for 16 h. Dilution of the mixture with Et₂O, washing of the organic phase with brine, and concentration under vacuum gave an oily residue consisting of silylated intermediate “H” (0.283 g, 88% yield), which was used in the next step without further purification. A solution of intermediate “H” (0.283 g, 0.821 mmol) in pyridine (1.5 mL) and CH₂Cl₂ (1.1 mL) was cooled to 0° C. and treated dropwise with methanesulfonyl chloride (MsCl, 0.1 mL, ˜1.5 mmol, ˜2 equiv.) and the mixture was stirred for 16 h at 0° C. Dilution of the mixture with Et₂O, washing of the organic phase with water and brine, and concentration under vacuum gave a crude residue that was purified by flash column chromatography (8:2 n-Hexane/EtOAc, 0.01% Et₃N) to afford intermediate “I” (0.254 g, 71% yield) as an oil. A solution of intermediate “I” (0.254 g, 0.579 mmol) in anhydrous THF (18 mL) was cooled to 0° C. and treated dropwise with a 1M solution of tetrabutylammonium fluoride (TBAF) in THF (0.4 mL, 0.6 mmol, 1 equiv.).

Stirring was continued for 40 min. at the same temperature. Dilution of the mixture with Et₂O, washing of the organic phase with brine, and concentration under vacuum gave a crude residue that was purified by flash column chromatography (1:1 n-Hexane/EtOAc, 0.01% Et₃N) to afford intermediate “J” (0.122 g, 63% yield) as an oil. A solution of intermediate “J” (0.087 g, 0.282 mmol) in CH₃CN (6 mL) was treated with t-BuOK (0.035 g, 0.54 mmol, 1.1 equiv.) and the mixture was stirred at room temperature for 30 minutes. Then, methyl 1-hydroxy-6-phenyl-4-(trifluoromethyl)-1H-indole-2-carboxylate (0.104 g, 0.309 mmol, 1.1 equiv.) was added and stirring was continued for 3 h at the same temperature. The mixture was diluted with CH₂Cl₂ and the organic phase was washed with brine and concentrated under vacuum. The crude residue was purified by flash column chromatography (7:3 n-Hexane/EtOAc, 0.1% Et₃N) to afford intermediate “K” (0.086 g, 51% yield) as a white solid. A solution of intermediate “K” (0.040 g, 0.073 mmol) in 0.3 mL of a 1:1 t-BuOH/acetone mixture was cooled to 0° C. and treated with 0.1 mL of a 50% w/v solution of N-methylmorpholin-N-oxide (NMO) in water and 0.1 mL of a 2.5% w/v solution of OsO₄ in t-BuOH. The resulting mixture was stirred at 0° C. for 8 h, then it was diluted with EtOAc and filtered through a 1 cm Celite® pad. Evaporation under vacuum of the filtrate gave a crude residue, which was purified by flash column chromatography (2:8 n-Hexane/EtOAc, 0.01% Et₃N) to afford intermediate “L” (0.020 g, 47% yield) as a white solid. Final deprotection step to remove the 2-tetrahydropyranyl

(THP) protecting group was achieved by treating a solution of intermediate “L” (0.040 g, 0.069 mmol) in absolute EtOH (0.5 mL) with pyridinium p-toluenesulfonate (PPTS) (0.0017 g, 0.0068 mmol, 0.1 equiv.) at 40° C. The resulting mixture was stirred at the same temperature for 20 h, then it was diluted with Et₂O. The organic phase was washed with a saturated aqueous solution of NaHCO₃ and with brine. After evaporation under vacuum of the organic phase, the resulting crude residue was recrystallized from n-Hexane/Et₂O, to afford 0.014 g (41% yield) methyl 1-(α-D-mannopyranosyl)oxy-6-phenyl-4-(trifluoromethyl)-1H-indole-2-carboxylate (Example 41) as a white solid.

### Example 51

A solution containing triphenylphosphine (84.1 mg, 0.320 mmol), ethanol (4.8 mL), toluene (4.8 mL) and Pd(OAc)₂ (14.4 mg, 0.0641 mmol) was stirred under nitrogen at room temperature for 10 min. Then, commercially available 5-bromo-1-fluoro-2-methyl-3-nitrobenzene (500 mg, 2.14 mmol), an aqueous sodium carbonate solution (4.8 mL, 2 M) and phenylboronic acid (417 mg, 3.42 mmol) were added and the resulting mixture was heated at 100° C. for 24 h under stirring in a sealed vial. The reaction mixture was diluted with water and extracted with EtOAc. The organic phase was washed with brine and concentrated under vacuum, leaving a crude residue that was purified by flash column chromatography (n-hexane) to afford the corresponding intermediate “M” as a white solid (500 mg, 91% yield) [Scheme 5, R³ and R⁵═H; ¹H NMR (CDCl₃): δ 2.51 (d, 3H, J=2.2 Hz), 7.39-7.71 (m, 6H), 7.98 (t, 1H, J=1.8 Hz)]. Subsequently, a solution of intermediate “M” (318 mg, 1.38 mmol) and dimethyl oxalate (812 mg, 6.88 mmol) in anhydrous THF (2.0 mL) was added dropwise under nitrogen to a cooled (0° C.) 30% solution of sodium methoxide in MeOH (1.3 mL). The resulting reddish suspension was stirred at room temperature for 18 h. The reaction mixture was then cooled to 0° C. and quenched with ice and 1N aqueous HCl until pH 5 was reached. Then the mixture was diluted with water and extracted with EtOAc. The organic phase was washed with brine and concentrated under vacuum evaporated to give a crude residue that was purified by flash column chromatography (n-hexane/EtOAc 8:2), affording the corresponding intermediate “N” (264 mg, 60% yield) as an yellow oily product [Scheme 5, R³ and R⁵═H. ¹H NMR (CDCl₃): δ 3.97 (s, 3H), 4.62 (s, 2H), 7.45-7.68 (m, 6H), 8.20 (s, 1H)]. Finally, intermediate “N” (145 mg, 0.457 mmol) was dissolved in dry DME (0.4 mL) and the resulting solution was added dropwise under nitrogen to a cooled (0° C.) solution of anhydrous SnCl₂ (217 mg, 1.14 mmol) in dry DME (0.5 mL) containing activated 4 Å molecular sieves. The reaction mixture was stirred at room temperature for 72 h, then it was filtered and concentrated under vacuum to afford a crude residue that was purified by flash chromatography (n-hexane/EtOAc 85:15) to give methyl 4-fluoro-1-hydroxy-6-phenyl-1H-indole-2-carboxylate (Example 51) as a light yellow solid (98.3 mg, 75% yield).

### Example 52

A solution of methyl 4-fluoro-1-hydroxy-6-phenyl-1H-indole-2-carboxylate (Example 51) (50.0 mg, 0.175 mmol) in a 1:1 mixture of THF/methanol (1.8 mL) was treated with 0.5 mL of a 2N aqueous solution of LiOH. The reaction mixture was stirred at room temperature for 22 h. The mixture was then partially concentrated under vacuum and, then, diluted with water and diethyl ether. The aqueous phase was separated, washed again with diethyl ether, and then treated with a 1N aqueous HCl solution and finally extracted with EtOAc. The organic phase was concentrated under vacuum to afford 4-fluoro-1-hydroxy-6-phenyl-1H-indole-2-carboxylic acid (Example 52) as an off-white solid (40.8 mg, 86%).

### Example 55

Precursor 6-(2,4-dichlorophenyl)-1-hydroxy-4-(trifluoromethyl)-1H-indole-2-carboxylic acid (180 mg, 0.461 mmol) [Minutolo, F.; Macchia, M.; Granchi, C.; Roy, S.; Giannaccini, G.; Lucacchini, A.; WO2011054525] was suspended in anhydrous CH₃CN (3 mL) and treated with CDI (74.8 mg, 0.461 mmol). The mixture was then heated to 50° C. until complete dissolution of the components. Then, benzylic alcohol (0.05 mL, 0.5 mmol) was added and the resulting mixture was heated to 65° C. for 5 hours. Most of the solvent was then removed under a nitrogen flux. The residue was extracted with EtOAc. The organic phase was washed with brine, dried over sodium sulfate and concentrated. The crude residue was purified by flash chromatography (n-hexane/EtOAc 85:15 or n-Hexane/Et₂O 8:2 to 7:3). In some cases, an additional trituration with n-hexane was needed for a better purification. Benzyl 6-(2,4-dichlorophenyl)-1-hydroxy-4-(trifluoromethyl)-1H-indole-2-carboxylate (Example 55) was so obtained as a yellow solid (66 mg, 30% yield).

### Characterization Data of Examples 1-64

### Example 1

¹H NMR (CDCl₃): δ 1.48 (t, 3H, J=7.1 Hz), 4.50 (q, 2H, J=7.1 Hz), 7.20 (bs, 1H), 7.36-7.55 (m, 3H), 7.65-7.73 (m, 3H), 7.92 (bs, 1H), 10.71 (bs, 1H). ¹³C NMR (CDCl₃): δ 14.43, 62.34, 102.07, 111.30, 116.24, 119.09 (q, J=5.5 Hz), 122.72, 124.16 (q, J=33.0 Hz), 124.54 (q, J=272.8 Hz), 127.54 (2C), 128.05, 129.14 (2C), 133.93, 138.41, 140.27, 164.14. MS (EI, 70 eV) m/z: 349 (M⁺, 87%), 333 (M⁺—O, 33%), 321 (M⁺—C₂H₄, 86%), 305 (M⁺—O—C₂H₄, 40%), 259 (M⁺—COOC₂H₅—OH, 100%), 190 (M⁺—COOC₂H₅—OH—CF₃, 71%).

### Example 2

¹H NMR (CD₃OD): δ 3.35-3.68 (m, 4H), 3.76-3.86 (m, 2H), 5.25 (d, 1H, J=7.7 Hz), 7.22 (qd, 1H, J=1.8, 0.9 Hz), 7.34-7.54 (m, 3H), 7.74-7.80 (m, 3H), 8.35 (bs, 1H). ¹³C NMR (CD₃OD): δ 62.40, 70.69, 73.76, 78.13, 78.44, 106.85, 109.94, 115.02, 118.42 (q, J=1.8 Hz), 120.02 (q, J=4.6 Hz), 124.33 (q, J=33.0 Hz), 125.81 (q, J=271.3 Hz), 128.39 (2C), 128.95, 130.06 (2C), 130.32, 139.84, 140.00, 141.15, 163.06. MS (ESI, negative) m/z: 482 (M−H⁺). [α]=+67.23 (c=0.98, MeOH).

### Example 3

¹H NMR (CD₃OD): δ 3.48-3.66 (m, 4H), 3.76-3.82 (m, 2H), 3.98 (s, 3H), 5.22 (d, 1H, J=7.7 Hz), 7.23 (qd, 1H, J=1.7, 1.0 Hz), 7.34-7.56 (m, 3H), 7.72-7.82 (m, 3H), 8.33 (bs, 1H). ¹³C NMR (CD₃OD): δ 52.95, 62.40, 70.81, 73.74, 78.01, 78.39, 106.78, 109.69, 115.02, 118.50 (q, J=1.2 Hz), 120.13 (q, J=4.6 Hz), 124.35 (q, J=32.0 Hz), 128.39 (2C), 125.90 (q, J=270.1 Hz), 128.99, 130.08 (2C), 130.20, 139.80, 140.15, 141.12, 162.00. MS (ESI, positive) m/z: 497 (M⁺). [α]=+62.88 (c=0.56, MeOH).

### Example 4

¹H NMR (CD₃OD): δ 1.71 (s, 3H), 2.00 (s, 3H), 2.03 (s, 3H), 2.15 (s, 3H), 3.88-4.02 (m, 2H), 4.24-4.35 (m, 1H), 5.16-5.53 (m, 3H), 5.73 (d, 1H, J=8.1 Hz), 7.21 (qd, 1H, J=1.8, 0.9 Hz), 7.35-7.55 (m, 3H), 7.65-7.76 (m, 3H), 8.16 (bs, 1H). ¹³C NMR (CD₃OD): δ 20.32, 20.54 (2C), 20.87, 62.82, 69.41, 71.20, 73.02, 73.82, 106.57, 107.39, 115.33, 118.61 (q, J=1.4 Hz), 120.18 (q, J=5.5 Hz), 124.25 (q, J=33.0 Hz), 125.76 (q, J=271.9 Hz), 128.32 (2C), 129.02, 130.14 (2C), 131.06, 140.02, 140.84, 141.30, 161.85, 171.10, 171.40 (2C), 171.90. MS (ESI, negative) m/z: 650 (M−H⁺). [α]=+39.94 (c=0.53, MeOH).

### Example 5

¹H NMR (CDCl₃): δ 1.75 (s, 3H), 2.03 (s, 3H), 2.06 (s, 3H), 2.19 (s, 3H), 3.75 (ddd, 1H, J=9.8, 4.3, 2.3 Hz), 3.94 (s, 3H), 3.96 (dd, 1H, J=12.3, 2.2 Hz), 4.31 (dd, 1H, J=12.5, 4.2 Hz), 5.18-5.50 (m, 3H), 5.57-5.63 (m, 1H), 7.30 (qd, 1H, J=1.6, 0.9 Hz), 7.38-7.52 (m, 3H), 7.59-7.66 (m, 2H), 7.73 (bs, 1H), 8.09 (bs, 1H). ¹³C NMR (CDCl₃): δ 20.35, 20.75, 20.79, 20.99, 52.20, 61.50, 68.15, 69.80, 72.08, 72.61, 105.38, 107.77, 114.41, 117.72 (q, J=1.8 Hz), 119.92 (q, J=4.6 Hz), 123.67 (q, J=33.9 Hz), 124.33 (q, J=271.9 Hz), 127.45 (2C), 128.11, 128.84, 129.13 (2C), 139.29, 140.22 (2C), 159.94, 169.49, 169.82, 170.04, 170.46. MS (ESI, negative) m/z: 664 (M−H⁺). [α]=+44.92 (c=1.01, CHCl₃).

### Example 6

¹HNMR (CD₃OD): δ 1.44 (t, 3H, J=7.1 Hz), 3.48-3.67 (m, 4H), 3.76-3.81 (m, 2H), 4.45 (q, 2H, J=7.1 Hz), 5.23 (d, 1H, J=7.5 Hz), 7.20 (qd, 1H, J=1.6, 0.7 Hz), 7.34-7.55 (m, 3H), 7.73-7.81 (m, 3H), 8.33 (bs, 1H). ¹³C NMR (DMSO-dd: δ 12.10, 60.84, 61.17, 69.34, 72.13, 76.06, 76.97, 103.61, 107.78, 113.37, 116.54, 118.51 (q, J=4.0 Hz), 121.85 (q, J=32.0 Hz), 124.12 (q, J=271.9 Hz), 127.05 (2C), 127.88, 128.94 (2C), 130.18, 136.93, 137.41, 138.88, 159.04. MS (ESI, positive) m/z: 512.2 (M+H⁺), 534.1 (M+Na⁺). [α]=+58.99 (c=0.556, MeOH).

### Example 7

¹HNMR (CDCl₃): δ 1.44 (t, 3H, J=7.1 Hz), 1.75 (s, 3H), 2.03 (s, 3H), 2.05 (s, 3H), 2.18 (s, 3H), 3.75 (ddd, 1H, J=9.7, 4.3, 2.2 Hz), 3.96 (dd, 1H, J=12.3, 2.4 Hz), 4.31 (dd, 1H, J=12.5, 4.3 Hz), 4.39 (q, 2H, J=7.1 Hz), 5.18-5.49 (m, 3H), 5.59-5.65 (m, 1H), 7.27-7.29 (m, 1H), 7.36-7.52 (m, 3H), 7.60-7.66 (m, 2H), 7.72 (bs, 1H), 8.09 (bs, 1H). ¹³C NMR (CDCl₃): δ 14.54, 20.33, 20.73 (2C), 20.93, 61.32, 61.57, 68.26, 69.90, 72.15, 72.68, 105.40, 107.55, 114.43, 117.78, 119.89 (q, J=4.6 Hz), 123.66 (q, J=33.0 Hz), 124.40 (q, J=271.9 Hz), 127.45 (2C), 128.09, 129.11 (2C), 129.38, 139.21, 140.23, 140.27, 159.55, 169.46, 169.78, 170.00, 170.40. [α]=+51.75 (c=0.572, CHCl₃).

### Example 8

¹H NMR (CDCl₃): δ 4.04 (s, 3H), 7.23 (bs, 1H), 7.34-7.36 (m, 2H), 7.50 (bs, 1H), 7.54 (pseudo-t, 1H, J=1.2 Hz), 7.78 (bs, 1H), 10.52 (bs, 1H). ¹H NMR (acetone-d₆): δ 3.95 (s, 3H), 7.19 (qd, 1H, J=1.7, 1.0 Hz), 7.53 (dd, 1H, J=8.2, 2.0 Hz), 7.59 (bs, 1H), 7.60 (d, 1H, J=8.2 Hz), 7.68 (d, 1H, J=2.0 Hz), 7.89 (bs, 1H), 10.91 (bs, 1H). ¹³C NMR (CDCl₃): δ 52.92, 102.34, 114.21, 116.49, 120.82 (q, J=5.5 Hz), 122.92, 123.61 (q, J=33.0 Hz), 124.30 (q, J=272.8 Hz), 127.49, 130.07, 132.35, 133.24, 133.55, 134.61, 135.12, 138.03, 164.25. MS (EI, 70 eV) m/z: 403 (M⁺, 53%), 389 (M⁺—CH₂, 100%), 373 (M⁺—O—CH₂, 13%), 327 (M⁺—COOCH₃—OH, 62%), 292 (M⁺—COOCH₃—OH—Cl, 53%), 258 (M⁺—COOCH₃—OH—CF₃, 13%).

### Example 9

¹H NMR (CDCl₃): δ 1.48 (t, 3H, J=7.1 Hz), 4.51 (q, 2H, J=7.1 Hz), 7.22 (qd, 1H, J=2.0, 0.9 Hz), 7.34-7.36 (m, 2H), 7.50 (bs, 1H), 7.54 (pseudo-t, 1H, J=1.2 Hz), 7.77 (bs, 1H), 10.71 (bs, 1H). ¹³C NMR (CDCl₃): δ 14.41, 62.45, 102.00, 114.18, 116.44, 120.75 (q, J=5.5 Hz), 122.99, 123.56 (q, J=32.0 Hz), 124.35 (q, J=271.9 Hz), 127.49, 130.07, 132.35, 133.06, 133.57, 134.57, 134.99, 138.08, 164.05. MS (EI, 70 eV) m/z: 417 (M⁺, 12%), 401 (M⁺—O, 46%), 389 (M⁺—C₂H₄, 33%), 373 (M⁺—O—C₂H₄, 52%), 258 (M⁺—COOC₂H₅—OH—CF₃, 100%).

### Example 10

¹H NMR (CD₃OD): δ 3.44-3.68 (m, 4H), 3.71-3.78 (m, 2H), 3.98 (s, 3H), 5.22 (d, 1H, J=7.5 Hz), 7.26 (qd, 1H, J=1.6, 0.9 Hz), 7.45 (dd, 1H, J=8.2, 2.0 Hz), 7.52 (d, 1H, J=0.4 Hz), 7.56-7.59 (m, 1H), 7.63 (dd, 1H, J=1.6, 0.4 Hz), 8.12 (bs, 1H). ¹³C NMR (DMSO-d₆): δ 52.16, 60.82, 69.33, 72.02, 76.12, 77.01, 103.50, 107.86, 116.40, 116.69, 120.67, 120.99 (q, J=32.7 Hz), 123.97 (q, J=270.0 Hz), 127.56, 129.09, 129.92, 132.34, 132.89, 133.35, 134.29, 136.10, 137.37, 159.35. [α]=+57.50 (c=0.45, acetone).

### Example 11

¹H NMR (CDCl₃): δ 1.75 (s, 3H), 2.02 (s, 3H), 2.04 (s, 3H), 2.17 (s, 3H), 3.73 (ddd, 1H, J=9.9, 3.8, 2.2 Hz), 3.94 (s, 3H), 3.96 (dd, 1H, J=12.6, 2.4 Hz), 4.23 (dd, 1H, J=12.5, 4.0 Hz), 5.14-5.48 (m, 3H), 5.59 (d, 1H, J=7.9 Hz), 7.30-7.35 (m, 3H), 7.52-7.54 (m, 2H), 7.88 (bs, 1H). ¹³C NMR (CDCl₃): δ 20.24, 20.66 (2C), 20.86, 52.18, 61.59, 68.33, 69.97, 72.32, 72.72, 105.38, 107.75, 117.07, 118.18, 121.72 (q, J=4.6 Hz), 123.28 (q, J=33.0 Hz), 124.27 (q, J=270.1 Hz), 127.49, 129.42, 129.96, 132.24, 133.66, 134.73, 136.10, 138.25, 139.47, 159.86, 169.35, 169.64, 169.93, 170.20. [α]=+23.80 (c=1.06, CHCl₃).

### Example 12

¹H NMR (CD₃OD): δ 1.45 (t, 3H, J=7.1 Hz), 3.45-3.82 (m, 6H), 4.46 (q, 2H, J=7.1 Hz), 5.23 (d, 1H, J=7.2 Hz), 7.24 (qd, 1H, J=2.0, 0.9 Hz), 7.42-7.64 (m, 4H), 8.11 (bs, 1H). ¹³C NMR (DMSO-d₆): δ 13.85, 60.88, 61.21, 69.44, 72.06, 76.10, 77.03, 103.34, 107.64, 116.30, 116.70, 120.66, 120.99 (q, J=33.9 Hz), 124.30 (q, J=267.0 Hz), 127.58, 129.10, 130.32, 132.36, 132.91, 133.36, 134.24, 135.97, 137.37, 158.93. MS (ESI, positive) m/z: 580.1 (M+H⁺), 602.1 (M+Na⁺). [α]=+45.22 (c=0.544, MeOH).

### Example 13

¹H NMR (CDCl₃): δ 1.44 (t, 3H, J=7.1 Hz), 1.75 (s, 3H), 2.02 (s, 3H), 2.04 (s, 3H), 2.16 (s, 3H), 3.73 (ddd, 1H, J=10.0, 3.9, 2.0 Hz), 3.96 (dd, 1H, J=12.4, 2.3 Hz), 4.23 (dd, 1H, J=12.5, 3.9 Hz), 4.40 (q, 2H, J=7.1 Hz), 5.15-5.47 (m, 3H), 5.61 (d, 1H, J=8.1 Hz), 7.29 (qd, 1H, J=1.8, 1.1 Hz), 7.30-7.38 (m, 2H), 7.51-7.54 (m, 2H), 7.88 (bs, 1H). ¹³C NMR (CDCl₃): δ 14.52, 20.30, 20.73 (2C), 20.92, 61.43, 61.50, 68.20, 69.86, 72.17, 72.59, 105.31, 107.46, 117.07, 118.11 (q, J=1.8 Hz), 121.63 (q, J=4.6 Hz), 123.12 (q, J=33.0 Hz), 124.26 (q, J=269.2 Hz), 127.45, 129.76, 129.91, 132.24, 133.60, 134.64, 135.92, 138.21, 139.45, 159.46, 169.40, 169.75, 169.98, 170.27. [α]=+23.54 (c=0.542, CHCl₃).

### Example 14

¹H NMR (CDCl₃): δ 4.00 (s, 3H), 7.08-7.33 (m, 1H), 7.54 (bs, 1H), 10.26 (bs, 1H). ¹³C NMR (CDCl₃): δ 52.87, 102.62, 117.29, 121.75, 122.05 (q, J=5.2 Hz), 122.62 (q, J=32.0 Hz), 125.68 (q, J=266.4 Hz), 126.83, 127.23 (2C), 127.36, 127.85 (2C), 130.20 (2C), 131.09 (2C), 132.04, 136.01, 138.80, 140.31, 164.21. MS (EI, 70 eV) m/z: 411 (M⁺, 18%), 397 (M⁺—CH₂, 11%), 395 (M⁺—O, 10%), 335 (M⁺—COOCH₃—OH, 99%), 266 (M⁺—COOCH₃—OH—CF₃, 100%).

### Example 15

¹H NMR (CDCl₃): δ 4.04 (s, 3H), 7.21 (qd, 1H, J=1.6, 1.1 Hz), 7.46 (AA′XX′, 2H, J_{AX}=8.8 Hz, J_{AA′/XX}=2.2 Hz), 7.62 (AA′XX′, 2H, J_{AX}=8.8 Hz, J_{AA′/XX}=2.2 Hz), 7.66 (bs, 1H), 7.89 (bs, 1H), 10.55 (bs, 1H). ¹³C NMR (CDCl₃): δ 52.89, 102.36, 111.23, 118.75 (q, J=5.5 Hz), 122.75, 124.36 (q, J=271.9 Hz), 124.37 (q, J=33.0 Hz), 125.18, 128.71 (2C), 129.31 (2C), 133.93, 134.32, 137.14, 138.63, 164.27. MS (EI, 70 eV) m/z: 369 (M⁺, 92%), 355 (M⁺—CH₂, 94%), 294 (M⁺—O—COOCH₃, 100%), 293 (M⁺—COOCH₃—OH, 45%), 258 (M⁺—COOCH₃—OH—Cl, 61%), 224 (M⁺—COOCH₃—OH —CF₃, 10%).

### Example 16

¹H NMR (CDCl₃): δ 2.66 (bs, 3H), 4.06 (s, 3H), 7.37-7.54 (m, 3H), 7.62-7.75 (m, 3H), 7.91 (bs, 1H), 10.68 (bs, 1H). MS (EI, 70 eV) m/z: 349 (M⁺, 65%), 335 (M⁺—CH₂, 62%), 333 (M⁺—O, 76%), 319 (M⁺—O—CH₂, 79%), 273 (M⁺—COOCH₃—OH, 100%), 204 (M⁺—COOCH₃—OH—CF₃, 33%).

### Example 17

¹H NMR (CDCl₃): δ 2.66 (bs, 3H), 4.07 (s, 3H), 7.45 (AA′XX′, 2H, J_{AX}=8.4 Hz, J_{AA′/XX}=2.3 Hz), 7.61 (AA′XX′, 2H, J_{AX}=8.6 Hz, J_{AA′/XX}=2.3 Hz), 7.69 (bs, 1H), 7.87 (bs, 1H), 10.74 (bs, 1H). ¹³C NMR (CDCl₃): δ 10.90, 52.85, 111.43, 115.45, 116.33, 118.96 (q, J=6.4 Hz), 121.12, 123.97 (q, J=33.0 Hz), 124.22 (q, J=271.9 Hz), 128.60 (2C), 129.27 (2C), 131.00, 134.19, 136.41, 138.45, 165.52. MS (EI, 70 eV) m/z: 383 (M⁺, 52%), 369 (M⁺—CH₂, 66%), 367 (M⁺—O, 23%), 353 (M⁺—O—CH₂, 79%), 307 (M⁺—COOCH₃—OH, 100%), 272 (M⁺—COOCH₃—OH—Cl, 20%), 238 (M⁺—COOCH₃—OH—CF₃, 16%).

### Example 18

¹H NMR (CDCl₃): δ 4.04 (s, 3H), 7.21 (qd, 1H, J=1.6, 0.9 Hz), 7.30-7.38 (m, 2H), 7.66 (bs, 1H), 7.69 (AA′XX′, 2H, J_{AX}=8.8 Hz, J_{AA′/XX}=1.9 Hz), 7.89 (bs, 1H), 10.56 (bs, 1H). ¹³C NMR (CDCl₃): δ 52.94, 102.29, 111.45, 116.38, 118.82 (q, J=5.5 Hz), 120.61 (q, J=255.6 Hz), 121.57 (2C), 122.70, 124.38 (q, J=33.0 Hz), 124.45 (q, J=270 Hz), 128.89 (2C), 133.81, 136.92, 138.90, 149.23, 164.29. MS (EI, 70 eV) m/z: 419 (M⁺, 19%), 405 (M⁺—CH₂, 38%), 389 (M⁺—O—CH₂, 11%), 343 (M⁺—COOCH₃—OH, 100%), 274 (M⁺—COOCH₃—OH—CF₃, 49%).

### Example 19

¹H NMR (CDCl₃): δ 4.05 (s, 3H), 7.22 (qd, 1H, J=1.6, 0.9 Hz), 7.27-7.31 (m, 1H), 7.47-7.68 (m, 4H), 7.91 (bs, 1H), 10.57 (bs, 1H). ¹³C NMR (CDCl₃): δ 52.89, 102.43, 111.61, 116.69, 118.78 (q, J=5.5 Hz), 120.11, 120.26, 120.72 (q, J=257.3 Hz), 123.08, 124.37 (q, J=271.9 Hz), 124.54 (q, J=33.0 Hz), 125.87, 130.49, 133.97, 136.81, 142.36, 150.07, 164.23. MS (EI, 70 eV) m/z: 419 (M⁺, 76%), 405 (M⁺—CH₂, 99%), 389 (M⁺—O—CH₂, 26%), 343 (M⁺—COOCH₃—OH, 100%), 274 (M⁺—COOCH₃—OH—CF₃, 36%).

### Example 20

¹H NMR (CDCl₃): δ 2.68 (q, 3H, J=1.3 Hz), 4.08 (s, 3H), 7.33-7.37 (m, 2H), 7.51-7.55 (m, 2H), 7.78 (bs, 1H), 10.70 (bs, 1H). ¹³C NMR (CDCl₃): δ 11.46 (q, J=5.0 Hz), 52.89, 114.52, 115.43, 116.47, 121.11 (q, J=6.4 Hz), 121.26, 123.24 (q, J=33.0 Hz), 124.16 (q, J=271.9 Hz), 127.47, 130.04, 132.29, 133.51, 133.86, 134.50, 134.59, 137.94, 165.47. MS (EI, 70 eV) m/z: 417 (M⁺, 46%), 403 (M⁺—CH₂, 99%), 387 (M⁺—O—CH₂, 21%), 341 (M⁺—COOCH₃—OH, 100%), 306 (M⁺—COOH—OH—Cl, 56%), 272 (M⁺—COOCH₃—OH—CF₃, 23%).

### Example 21

¹H NMR (CDCl₃): δ 4.05 (s, 3H), 7.21 (qd, 1H, J=1.6, 0.9 Hz), 7.50 (dd, 1H, J=8.4, 2.0 Hz), 7.57 (d, 1H, J=8.4 Hz), 7.63 (bs, 1H), 7.76 (d, 1H, J=1.8 Hz), 7.88 (bs, 1H), 10.59 (bs, 1H). ¹³C NMR (CDCl₃): δ 53.00, 102.23, 111.39, 116.56, 118.44 (q, J=5.5 Hz), 122.84, 124.21 (q, J=270 Hz), 124.52 (q, J=33.0 Hz), 126.65, 129.24, 131.04, 132.35, 133.31, 133.64, 135.75, 140.12, 164.27. MS (EI, 70 eV) m/z: 403 (M⁺, 83%), 389 (M⁺—CH₂, 100%), 387 (M⁺—O, 22%), 373 (M⁺—O—CH₂, 21%), 327 (M⁺—COOCH₃—OH, 90%), 292 (M⁺—COOCH₃—OH—Cl, 72%), 257 (M⁺—COOCH₃—OH—CF₃, 24%).

### Example 22

¹H NMR (CDCl₃): δ 1.02 (t, 3H, J=7.2 Hz), 1.51 (sextet, 2H, J=7.3 Hz), 1.83 (quintet, 2H, J=7.1 Hz), 4.48 (t, 2H, J=6.7 Hz) 7.18 (bs, 1H), 7.36-7.55 (m, 3H), 7.65-7.72 (m, 3H), 7.92 (bs, 1H), 10.74 (bs, 1H). ¹³C NMR (CDCl₃): δ 13.85, 19.37, 30.88, 66.14, 102.03, 111.30, 116.27, 119.10 (q, J=4.6 Hz), 122.81, 124.16 (q, J=33.0 Hz), 124.57 (q, J=271.9 Hz), 127.52 (2C), 128.05, 129.13 (2C), 134.01, 138.41, 140.27, 164.20. MS (EI, 70 eV) m/z: 377 (M⁺, 37%), 361 (M⁺—O, 21%), 321 (M⁺—C₄H₈, 100%), 305 (M⁺—O—C₄H₈, 37%), 259 (M⁺—COOC₄H₉—OH, 56%), 190 (M⁺—COOC₄H₉—OH—CF₃, 40%).

### Example 23

¹H NMR (CDCl₃): δ 1.45 (d, 6H, J=6.4 Hz), 5.37 (heptet, 1H, J=6.3 Hz), 7.17 (bs, 1H), 7.39-7.54 (m, 3H), 7.66-7.73 (m, 3H), 7.92 (bs, 1H), 10.88 (bs, 1H). ¹³C NMR (CDCl₃): δ 22.04 (2C), 70.55, 101.70, 111.21, 116.04, 118.94 (q, J=4.6 Hz), 122.83, 123.96 (q, J=33.0 Hz), 124.49 (q, J=271.9 Hz), 127.47 (2C), 127.98, 129.09 (2C), 133.72, 138.14, 140.18, 163.78. MS (EI, 70 eV) m/z: 363 (M⁺, 23%), 347 (M⁺—O, 13%), 321 (M⁺—C₃H₆, 100%), 305 (M⁺—O—C₃H₆, 31%), 259 (M⁺—COOC₃H₇—OH, 52%), 190 (M⁺—COOC₃H₇—OH—CF₃, 42%).

### Example 24

¹H NMR (acetone-d₆): δ 7.14 (qd, 1H, J=1.8, 0.7 Hz), 7.44-7.52 (m, 2H), 7.78 (bs, 1H), 7.95 (AA′XX′, 2H, J_{AX}; =8.9 Hz, J_{AA′/XX}=2.4 Hz), 8.06 (bs, 1H). ¹³C NMR (acetone-d₆): δ 102.20, 112.56, 117.35, 118.82 (q, J=5.0 Hz), 121.42 (q, J=255.4 Hz), 122.34 (2C), 123.81 (q, J=33.0 Hz), 125.56 (q, J=271.0 Hz), 129.13, 129.91 (2C), 136.38, 136.54, 140.09, 149.60, 162.49. MS (EI, 70 eV) m/z: 405 (M⁺, 30%), 389 (M⁺—O, 100%), 343 (M⁺—COOH—OH, 61%), 274 (M⁺—COOH—OH—CF₃, 35%).

### Example 25

¹H NMR (acetone-dd: δ 7.20 (bs, 1H), 7.36-7.48 (m, 1H), 7.68 (t, 1H, J=8.0 Hz), 7.78-7.92 (m, 3H), 8.13 (bs, 1H). ¹³C NMR (acetone-d₆): δ 103.55, 112.91, 117.77, 119.05 (q, J=4.6 Hz), 120.84, 121.01, 121.43 (q, J=250 Hz), 124.83 (q, J=33.0 Hz), 125.53 (q, J=271.9 Hz), 127.14, 129.11, 131.70, 136.83, 137.48, 143.24, 150.60, 161.45. MS (EI, 70 eV) m/z: 405 (M⁺, 14%), 389 (M⁺—O, 100%), 343 (M⁺—COOH—OH, 41%), 274 (M⁺—COOH—OH—CF₃, 15%).

### Example 26

¹H NMR (acetone-d₆): δ 2.68 (q, 3H, J=1.7 Hz), 7.52 (dd, 1H, J=8.2, 2.0 Hz), 7.59 (bs, 1H), 7.60 (d, 1H, J=7.9 Hz), 7.67 (d, 1H, J=1.8 Hz), 7.87 (bs, 1H). ¹³C NMR (acetone-d₆): δ 11.49 (q, J=5.4 Hz), 114.27, 115.64, 117.82, 121.09 (q, J=6.5 Hz), 122.78 (q, J=32.0 Hz), 125.32 (q, J=271.0 Hz), 127.22, 128.46, 130.31, 133.74, 133.90, 134.34, 134.74, 136.39, 139.03, 163.47. MS (EI, 70 eV) m/z: 403 (M⁺, 41%), 387 (M⁺—O, 100%), 341 (M⁺—COOH—OH, 49%), 306 (M⁺—COOH—OH—Cl, 26%).

### Example 27

¹H NMR (acetone-d₆): δ 7.16 (bs, 1H), 7.69 (d, 1H, J=8.4 Hz), 7.80 (bs, 1H), 7.81 (dd, 1H, J=8.4, 2.2 Hz), 8.04 (d, 1H, J=2.0 Hz), 8.10 (bs, 1H). ¹³C NMR (acetone-d₆): δ 102.64, 112.76, 117.68, 118.57 (q, J=5.4 Hz), 123.93 (q, J=32.0 Hz), 125.47 (q, J=271.0 Hz), 128.07, 129.24, 129.99, 131.84, 132.08, 133.30, 135.36, 136.76, 141.37, 162.16. MS (EI, 70 eV) m/z: 389 (M⁺, 65%), 373 (M⁺—O, 100%), 327 (M⁺—COOH—OH, 74%), 292 (M⁺—COOH —OH—Cl, 60%), 257 (M⁺—COOH—OH—CF₃, 22%).

### Example 28

¹H NMR (CDCl₃): δ 1.01 (t, 3H, J=7.3 Hz), 1.49 (sextet, 2H, J=7.5 Hz), 1.75 (s, 3H), 1.78 (pentet, 2H, J=6.7 Hz), 2.03 (s, 3H), 2.06 (s, 3H), 2.18 (s, 3H), 3.75 (ddd, 1H, J=10.1, 4.4, 2.4 Hz), 3.96 (dd, 1H, J=12.4, 2.2 Hz), 4.27-4.40 (m, 3H), 5.19-5.49 (m, 3H), 5.62 (d, 1H, J=7.7 Hz), 7.25 (bs, 1H), 7.35-7.51 (m, 3H), 7.61-7.66 (m, 2H), 7.72 (bs, 1H), 8.09 (bs, 1H). ¹³C NMR (CDCl₃): δ 13.90, 19.44, 20.31, 20.72 (2C), 20.92, 30.98, 61.63, 65.18, 68.35, 69.95, 72.21, 72.75, 105.46, 107.46, 114.43, 117.80, 119.90 (q, J=4.6 Hz), 123.70 (q, J=33.0 Hz), 124.45 (q, J=272.8 Hz), 127.47 (2C), 128.09, 129.13 (2C), 129.42, 139.23, 139.41, 140.25, 159.62, 169.44, 169.77, 169.97, 170.37.

### Example 29

¹H NMR (CD₃OD): δ 1.03 (t, 3H, J=7.2 Hz), 1.50 (sextet, 2H, J=7.3 Hz), 1.82 (quintet, 2H, J=6.9 Hz), 3.49-3.65 (m, 4H), 3.76-3.82 (m, 2H), 4.40 (t, 2H, J=6.6 Hz), 5.20 (d, 1H, J=7.7 Hz), 7.18 (bs, 1H), 7.40-7.54 (m, 3H), 7.76-7.80 (m, 3H), 8.32 (bs, 1H). ¹³C NMR (DMSO-dd: δ 13.46, 18.62, 19.94, 60.88, 64.81, 69.38, 72.09, 76.06, 76.96, 103.41, 107.82, 113.21, 116.58, 118.50 (q, J=4.2 Hz), 121.88 (q, J=33.0 Hz), 124.95 (q, J=273.1 Hz), 127.01 (2C), 127.88, 128.94 (2C), 130.22, 136.64, 137.37, 138.86, 159.20.

### Example 30

¹H NMR (CDCl₃): δ 1.39 (d, 3H, J=6.8 Hz), 1.42 (d, 3H, J=6.6 Hz), 1.75 (s, 3H), 2.03 (s, 3H), 2.06 (s, 3H), 2.18 (s, 3H), 3.75 (ddd, 1H, J=9.8, 4.1, 2.1 Hz), 3.95 (dd, 1H, J=12.5, 2.1 Hz), 4.31 (dd, 1H, J=12.5, 4.3 Hz), 5.18-5.50 (m, 4H), 5.64 (d, 1H, J=7.3 Hz), 7.24 (bs, 1H), 7.35-7.51 (m, 3H), 7.60-7.66 (m, 2H), 7.72 (bs, 1H), 8.09 (bs, 1H). ¹³C NMR (CDCl₃): δ 20.30, 20.72 (2C), 20.92, 22.14 (2C), 61.66, 68.42, 69.09, 70.02, 72.23, 72.81, 105.42, 107.30, 114.41, 117.82, 119.86 (q, J=5.5 Hz), 123.69 (q, J=32.7 Hz), 124.48 (q, J=272.8 Hz), 127.49 (2C), 128.07, 129.13 (2C), 129.87, 139.16, 140.23, 140.36, 159.12, 169.44, 169.75, 169.95, 170.35.

### Example 31

¹H NMR (CD₃OD): δ 1.43 (d, 3H, J=6.2 Hz), 1.44 (d, 3H, J=6.6 Hz), 3.49-3.85 (m, 6H), 5.23 (d, 1H, J=7.5 Hz), 5.30 (septet, 1H, J=6.2 Hz), 7.16 (bs, 1H), 7.36-7.55 (m, 3H), 7.75-7.81 (m, 3H), 8.32 (bs, 1H). ¹³C NMR (DMSO-d₆): δ 21.31, 21.44, 60.92, 69.07, 69.49, 72.15, 76.10, 76.97, 103.39, 107.55, 113.17, 116.49, 118.51 (q, J=4.0 Hz), 121.82 (q, J=33.0 Hz), 125.00 (q, J=272.1 Hz), 126.97 (2C), 127.81, 128.89 (2C), 130.47, 136.70, 137.30, 138.86, 158.57.

### Example 32

¹H NMR (CDCl₃): δ 1.45 (d, 6H, J=6.2 Hz), 5.37 (septet, 1H, J=6.3 Hz), 7.19 (qd, 1H, J=1.6, 0.9 Hz), 7.34-7.36 (m, 2H), 7.48-7.51 (m, 1H), 7.54 (t, 1H, J=1.3 Hz), 7.77 (bs, 1H), 10.87 (bs, 1H). ¹³C NMR (CDCl₃): δ 22.04 (2C), 70.75, 101.74, 114.16, 116.44, 120.70 (q, J=5.5 Hz), 123.26, 123.56 (q, J=32.5 Hz), 124.40 (q, J=271.9 Hz), 127.49, 130.07, 132.37, 132.99, 133.60, 134.57, 134.88, 138.14, 163.76.

### Example 33

¹H NMR (CDCl₃): δ 1.01 (t, 3H, J=7.2 Hz), 1.51 (sextet, 2H, J=7.3 Hz), 1.82 (quintet, 2H, J=7.1 Hz), 4.45 (t, 2H, J=6.7 Hz), 7.20 (qd, 1H, J=1.6, 0.9 Hz), 7.34-7.36 (m, 2H), 7.50 (bs, 1H), 7.54 (pseudo-t, 1H, J=1.2 Hz), 7.77 (bs, 1H), 10.75 (bs, 1H). ¹³C NMR (CDCl₃): δ 13.83, 19.35, 30.86, 66.25, 102.00, 114.20, 116.54, 120.80 (q, J=4.6 Hz), 123.15, 123.65 (q, J=33.3 Hz), 124.40 (q, J=271.9 Hz), 127.49, 130.09, 132.35, 133.20, 133.64, 134.62, 135.04, 138.14, 164.10.

### Example 34

¹H NMR (CDCl₃): δ 4.04 (s, 3H), 7.23 (bs, 1H), 7.39-7.55 (m, 4H), 7.58 (bs, 1H), 7.83 (bs, 1H), 10.51 (bs, 1H). ¹³C NMR (CDCl₃): δ 52.83, 102.45, 114.01, 116.51, 120.61 (q, J=257.3 Hz), 120.85 (q, J=4.6 Hz), 121.59, 123.01, 123.80 (q, J=33.0 Hz), 124.37 (q, J=271.9 Hz), 127.32, 129.47, 131.78, 133.64, 133.86, 134.30, 146.53, 164.27.

### Example 35

¹H NMR (acetone-d₆): δ 7.22 (bs, 1H), 7.50-7.74 (m, 5H), 7.94 (bs, 1H), 10.70 (bs, 1H). ¹³C NMR (acetone-d₆): δ 103.62, 115.22, 117.57, 120.04 (q, J=4.6 Hz), 121.46 (q, J=256.4 Hz), 122.50, 123.59 (q, J=32.5 Hz), 125.58 (q, J=271.0 Hz), 128.77, 129.04, 130.64, 132.86, 134.08, 135.12, 137.07, 147.05, 161.50.

### Example 36

¹H NMR (CDCl₃): δ 4.04 (s, 3H), 7.23 (bs, 1H), 7.26-7.32 (m, 2H), 7.50-7.55 (m, 2H), 7.78 (bs, 1H), 10.52 (bs, 1H). ¹³C NMR (CDCl₃): δ 52.91, 102.42, 114.18, 116.53, 120.85 (q, J=4.6 Hz), 123.01, 123.61 (q, J=33.4 Hz), 124.33 (q, J=271.9 Hz), 127.42, 129.73, 130.18, 131.49, 133.30, 134.08, 136.12, 141.80, 164.21.

### Example 37

¹H NMR (acetone-d₆): δ 7.22 (bs, 1H), 7.44-7.54 (m, 2H), 7.59 (bs, 1H), 7.65-7.71 (m, 1H), 7.88 (bs, 1H). ¹³C NMR (acetone-d₆): δ 103.58, 115.44, 117.69, 121.12 (q, J=5.5 Hz), 123.38 (q, J=33.0 Hz), 125.54 (q, J=271.0 Hz), 128.96, 129.11, 131.04, 131.15, 131.60, 134.12, 136.39, 136.80, 142.82, 161.45. MS (EI, 70 eV) m/z: 389 (M⁺, 100%), 373 (M⁺—O0, 11%), 327 (M⁺—COOH—OH, 42%), 292 (M⁺—COOH—OH—Cl, 59%), 257 (M⁺—COOH—OH—CF₃, 29%).

### Example 38

¹H NMR (CDCl₃): δ 4.05 (s, 3H), 7.24 (bs, 1H), 7.32 (dd, 1H, J=8.2, 2.7 Hz), 7.41-7.47 (m, 2H), 7.52 (bs, 1H), 7.79 (bs, 1H), 10.52 (bs, 1H). ¹³C NMR (CDCl₃): δ 52.91, 102.42, 114.27, 116.69, 120.74 (q, J=4.6 Hz), 123.14, 123.78 (q, J=33.9 Hz), 124.34 (q, J=272.8 Hz), 129.27, 131.22, 131.37, 131.44, 133.04, 133.31, 135.06, 140.98, 164.25.

### Example 39

¹H NMR (acetone-d₆): δ 7.23 (bs, 1H), 7.46-7.54 (m, 1H), 7.58-7.68 (m, 3H), 7.93 (bs, 1H). ¹³C NMR (acetone-d₆): δ 103.51, 115.55, 117.68, 121.02 (q, J=4.6 Hz), 123.30 (q, J=33.0 Hz), 125.47 (q, J=271.0 Hz), 129.18, 130.18, 131.73, 132.30 (2C), 133.52, 135.17, 136.76, 141.89, 161.34. MS (EI, 70 eV) m/z: 389 (M⁺, 100%), 373 (M⁺—O, 43%), 327 (M⁺—COOH—OH, 60%), 292 (M⁺—COOH—OH—Cl, 77%), 257 (M⁺—COOH—OH—CF₃, 43%).

### Example 40

¹H NMR (CD₃OD): δ 3.66 (dd, 1H, J=10.8, 6.1 Hz), 3.75-3.85 (m, 2H), 3.92 (dd, 1H, J=6.1, 1.3 Hz), 3.98 (s, 3H), 4.03 (dd, 1H, J=8.3, 3.4 Hz), 4.11 (t, 1H, J=3.6 Hz), 5.47 (d, 1H, J=8.3 Hz), 7.23 (qd, 1H, J=1.8, 0.9 Hz), 7.38-7.44 (m, 1H), 7.45-7.54 (m, 2H), 7.72-7.79 (m, 3H), 8.34 (bs, 1H). ¹³C NMR (CD₃OD): δ 52.36, 61.90, 68.60, 70.39, 73.20, 75.51, 106.73, 108.79, 115.20, 118.41 (q, J=1.3 Hz), 120.09 (q, J=4.6 Hz), 124.35 (q, J=32.3 Hz), 125.90 (q, J=271.7 Hz), 128.41 (2C), 129.05, 129.78, 130.11 (2C), 139.99, 140.07, 141.18, 162.23. [α]=+57.71 (c=0.35, MeOH).

### Example 41

¹H NMR (CD₃OD): δ 3.83-3.89 (m, 4H), 3.96 (s, 3H), 4.16-4.28 (m, 1H), 4.57-4.63 (m, 1H), 5.57 (d, 1H, J=2.0 Hz), 7.23 (qd, 1H, J=1.8, 0.9 Hz), 7.37-7.56 (m, 3H), 7.69-7.79 (m, 3H), 8.04 (s, 1H). ¹³C NMR (CD₃OD): δ 52.77, 62.65, 67.90, 70.53, 72.39, 77.93, 106.89, 111.20, 113.58, 118.30 (q, J=1.7 Hz), 120.21 (q, J=4.9 Hz), 124.75 (q, J=32.9 Hz), 125.20 (q, J=271.0 Hz), 128.54 (2C), 129.14, 129.30, 130.16 (2C), 139.24, 140.50, 141.08, 161.32. [α]=+18.14 (c=0.95, MeOH).

### Example 42

¹H NMR (CDCl₃): δ 4.05 (s, 3H), 7.22 (bs, 1H), 7.39 (t, 1H, J=1.8 Hz), 7.55 (d, 2H, J=1.8 Hz), 7.62 (bs, 1H), 7.88 (bs, 1H), 10.56 (bs, 1H). ¹³C NMR (CDCl₃): δ 52.96, 102.34, 111.70, 116.85, 118.47 (q, J=5.5 Hz), 123.21, 124.65 (q, J=33.0 Hz), 124.24 (q, J=271.9 Hz), 125.99 (2C), 127.96, 133.73, 135.55, 135.77 (2C), 143.20, 164.20.

### Example 43

¹H NMR (acetone-d₆): δ 7.20 (bs, 1H), 7.53 (t, 1H, J=1.6 Hz), 7.84-7.87 (m, 3H), 8.16 (bs, 1H). ¹³C NMR (acetone-d₆): δ 103.49, 113.22, 117.99, 119.01 (q, J=4.6 Hz), 124.16 (q, J=33.0 Hz), 125.48 (q, J=272.0 Hz), 126.91 (2C), 128.20, 129.28, 135.56, 136.16 (2C), 137.34, 144.33, 161.32. MS (EI, 70 eV) m/z: 389 (M⁺, 100%), 373 (M⁺—O, 80%), 327 (M⁺—COOH—OH, 78%), 292 (M⁺—COOH—OH —Cl, 83%), 257 (M⁺—COOH—OH—CF₃, 83%).

### Example 44

¹H NMR (CDCl₃): δ 1.39 (d, 3H, J=6.4 Hz), 1.42 (d, 3H, J=6.3 Hz), 1.75 (s, 3H), 2.02 (s, 3H), 2.04 (s, 3H), 2.16 (s, 3H), 3.73 (ddd, 1H, J=9.9, 3.8, 2.2 Hz), 3.96 (dd, 1H, J=12.5, 2.4 Hz), 4.23 (dd, 1H, J=12.3, 4.0 Hz), 5.14-5.48 (m, 4H), 5.62 (d, 1H, J=7.9 Hz), 7.24-7.31 (m, 2H), 7.35 (dd, 1H, J=8.2, 1.8 Hz), 7.50-7.54 (m, 2H), 7.88 (bs, 1H). ¹³C NMR (CDCl₃): δ 20.30, 20.73 (2C), 20.93, 22.14 (2C), 61.56, 68.26, 69.24, 69.91, 72.19, 72.64, 105.29, 107.18, 117.04, 118.15, 121.60 (q, J=4.6 Hz), 123.10 (q, J=33.0 Hz), 124.24 (q, J=272.8 Hz), 127.45, 129.93, 130.20, 132.26, 133.64, 134.64, 135.83, 138.27, 139.43, 159.04, 169.40, 169.77, 169.98, 170.28.

### Example 45

¹H NMR (CD₃OD): δ 1.43 (d, 3H, J=6.2 Hz), 1.44 (d, 3H, J=6.2 Hz), 3.41-3.82 (m, 6H), 5.23 (d, 1H, J=7.5 Hz), 5.31 (septet, 1H, J=6.2 Hz), 7.13-7.19 (m, 1H), 7.41-7.63 (m, 4H), 8.11 (bs, 1H). ¹³C NMR (CD₃OD): δ 22.07, 22.13, 62.58, 71.10, 73.82, 78.06, 78.33, 78.55, 104.81, 106.21, 109.47, 117.75, 118.77, 122.23 (q, J=4.0 Hz), 123.65 (q, J=33.0 Hz), 125.47 (q, J=272.0 Hz), 128.62, 130.65, 131.06, 133.90, 134.43, 136.84, 138.82, 139.42, 161.09.

### Example 46

¹H NMR (CDCl₃): δ 1.00 (t, 3H, J=7.2 Hz), 1.49 (sextet, 2H, J=7.2 Hz), 1.75 (s, 3H), 1.78 (quintet, 2H, J=7.0 Hz), 2.02 (s, 3H), 2.04 (s, 3H), 2.16 (s, 3H), 3.72 (ddd, 1H, J=9.8, 3.9, 2.1 Hz), 3.96 (dd, 1H, J=12.8, 2.2 Hz), 4.23 (dd, 1H, J=12.4, 3.9 Hz), 4.33 (t, 2H, J=6.7 Hz), 5.14-5.47 (m, 3H), 5.61 (d, 1H, J=7.7 Hz), 7.26-7.31 (m, 2H), 7.35 (dd, 1H, J=8.3, 1.9 Hz), 7.51-7.54 (m, 2H), 7.88 (bs, 1H). ¹³C NMR (CDCl₃): 13.89, 19.44, 20.28, 20.72 (2C), 20.90, 30.97, 61.56, 65.27, 68.26, 69.90, 72.21, 72.63, 105.35, 107.35, 117.05, 118.11, 121.62 (q, J=4.0 Hz), 123.17 (q, J=32.0 Hz), 124.28 (q, J=272.0 Hz), 127.45, 129.80, 129.93, 132.24, 133.63, 134.66, 135.94, 138.25, 139.45, 159.53, 169.38, 169.73, 169.95, 170.26.

### Example 47

¹H NMR (CD₃OD): δ 0.94 (t, 3H, J=7.2 Hz), 1.28-1.59 (m, 4H), 3.46-3.58 (m, 4H), 3.72 (dd, 1H, J=11.6, 4.5 Hz), 3.81 (dd, 1H, J=11.7, 2.6 Hz), 4.41 (t, 2H, J=6.6 Hz), 5.24 (d, 1H, J=7.7 Hz), 7.24 (bs, 1H), 7.45 (dd, 1H, J=8.2, 2.0 Hz), 7.55 (d, 1H, J=8.2 Hz), 7.57 (bs, 1H), 7.62 (d, 1H, J=2.0 Hz), 8.14 (bs, 1H). ¹³C NMR (CD₃OD): δ 14.20, 20.03, 35.82, 62.53, 62.69, 70.85, 73.76, 78.17, 78.53, 106.48, 109.58, 109.92, 117.84, 118.77, 122.12 (q, J=4.0 Hz), 123.54 (q, J=33.0 Hz), 125.48 (q, J=272.5 Hz), 128.62, 130.63, 133.92, 134.42, 135.51, 136.67, 138.97, 139.50, 163.13.

### Example 48

¹H NMR (CDCl₃): δ 5.46 (s, 2H), 7.23 (bs, 1H), 7.36-7.53 (m, 8H), 7.65-7.72 (m, 3H), 7.92 (bs, 1H), 10.51 (bs, 1H). ¹³C NMR (CDCl₃): δ 67.80, 102.62, 111.32, 116.27, 119.23 (q, J=4.6 Hz), 121.79, 122.72, 124.24 (q, J=32.0 Hz), 124.52 (q, J=272.0 Hz), 127.52 (2C), 128.09, 128.62 (2C), 128.94 (2C), 129.13 (2C), 134.21, 134.95, 138.61, 140.23, 163.80.

### Example 49

¹H NMR (CDCl₃): δ 0.90 (s, 9H), 1.08-1.57 (m, 5H), 1.84-1.98 (m, 2H), 2.13-2.26 (m, 2H), 4.99 (tt, 1H, J=11.5, 6.9 Hz), 7.17 (bs, 1H), 7.36-7.54 (m, 3H), 7.66-7.72 (m, 3H), 7.92 (bs, 1H), 10.88 (bs, 1H). ¹³C NMR (CDCl₃): δ 25.78 (2C), 27.82 (3C), 29.91, 32.33 (2C), 47.34, 75.41, 101.83, 111.28, 116.29, 119.08 (q, J=4.0 Hz), 123.08, 123.46 (q, J=32.0 Hz), 124.60 (q, J=270.0 Hz), 127.54 (2C), 128.01, 129.13 (2C), 133.88, 138.30, 140.34, 163.87.

### Example 50

¹H NMR (CDCl₃): δ 0.90 (s, 9H), 1.07-1.54 (m, 5H), 1.86-1.99 (m, 2H), 2.15-2.27 (m, 2H), 4.99 (tt, 1H, J=11.4, 6.9 Hz), 7.18 (bs, 1H), 7.35 (d, 1H, J=1.3 Hz), 7.49 (bs, 1H), 7.54 (t, 1H, J=1.1 Hz), 7.77 (bs, 1H), 10.89 (bs, 1H).

### Example 51

¹H NMR (CDCl₃): δ 4.02 (s, 3H), 7.07 (dd, 1H, J=11.5, 1.3 Hz), 7.12 (bs, 1H), 7.33-7.54 (m, 4H), 7.62-7.69 (m, 2H), 10.42 (bs, 1H). ¹³C NMR (CDCl₃): δ 52.63, 100.18, 103.84 (d, J=3.7 Hz), 105.27 (d, J=20.1 Hz), 110.64 (d, J=24.7 Hz), 121.86, 127.45 (2C), 127.89, 129.00 (2C), 135.77 (d, J=11.0 Hz), 140.52 (d, J=10.1 Hz), 140.62, 157.29 (d, J=250.9 Hz), 164.27. MS (EI, 70 eV) m/z: 285 (M⁺, 41%), 271 (M⁺—CH₂, 46%), 255 (M⁺—O—CH₂, 10%), 208 (M⁺—CH₂—CO₂—F, 100%).

### Example 52

¹H NMR (acetone-d₆): δ 7.15 (bs, 1H) 7.20 (d, 1H, J=11.9 Hz), 7.34-7.56 (m, 3H), 7.62 (bs, 1H), 7.73-7.79 (m, 2H). ¹³C NMR (acetone-d₆): δ 101.38, 104.64 (d, J=3.7 Hz), 105.48 (d, J=20.1 Hz), 111.26 (d, J=24.7 Hz), 127.38, 128.02 (2C), 128.58, 129.80 (2C), 139.20 (d, J=11.0 Hz), 140.56 (d, J=8.2 Hz), 141.22, 157.83 (d, J=250.0 Hz), 161.79. MS (EI, 70 eV) m/z: 271 (M⁺, 100%), 255 (M⁺—O, 28%), 208 (M⁺—CO₂—F, 59%).

### Example 53

¹H NMR (CDCl₃): δ 4.03 (s, 3H), 6.87 (dd, 1H, J=11.2, 1.1 Hz), 7.14 (bs, 1H), 7.32-7.38 (m, 3H), 7.51 (bs, 1H), 10.42 (s, 1H). ¹³C NMR (CDCl₃): δ 52.80, 100.05, 106.71 (d, J=3.7 Hz), 107.23 (d, J=19.2 Hz), 110.84 (d, J=23.8 Hz), 121.99, 127.32, 130.00, 132.31, 133.50, 134.32, 134.83 (d, J=11.0 Hz), 136.95 (d, J=8.2 Hz), 138.50, 156.48 (d, J=251.8 Hz), 164.25. MS (EI, 70 eV) m/z: 353 (M⁺, 34%), 339 (M⁺—CH₂, 48%), 277 (M⁺—CH₂—CO₂—H₂O, 10%), 242 (M⁺—CH₂—CO₂—H₂O—Cl, 100%).

### Example 54

¹H NMR (DMSO-d₆): δ 6.98 (d, 1H, J=11.5 Hz), 7.07 (bs, 1H), 7.30 (bs, 1H), 7.52-7.54 (m, 2H), 7.77 (bs, 1H). ¹³C NMR (acetone-d₆): δ 101.29, 107.59 (d, J=20.1 Hz), 107.79, 111.50 (d, J=23.8 Hz), 127.82, 128.35, 130.31, 133.68, 133.92, 134.65, 137.00 (d, J=8.2 Hz), 138.38 (d, J=11.0 Hz), 139.58, 156.96 (d, J=250.0 Hz), 161.54. MS (EI, 70 eV) m/z: 339 (M⁺, 70%), 323 (M⁺—O, 46%), 242 (M⁺—CO₂—H₂O—Cl, 100%).

### Example 55

¹H NMR (CDCl₃): δ 5.47 (s, 2H), 7.24-7.26 (m, 1H), 7.33-7.37 (m, 2H), 7.40-7.55 (m, 7H), 7.77 (bs, 1H), 10.53 (bs, 1H). ¹³C NMR (CDCl₃): δ 67.91, 102.56, 114.23, 116.54, 120.89 (q, J=4.6 Hz), 123.04, 123.70 (q, J=33.0 Hz), 124.33 (q, J=271.9 Hz), 127.49, 128.29, 128.64 (2C), 128.96 (2C), 130.11, 132.33, 133.40, 133.62, 134.66, 134.86, 135.23, 138.08, 163.72.

### Example 56

¹HNMR (CDCl₃): δ 5.51 (s, 2H), 7.26-7.28 (m, 1H), 7.33-7.68 (m, 13H), 7.78 (bs, 1H), 10.54 (bs, 1H).

### Example 57

¹HNMR (CDCl₃): δ 1.86-2.40 (m, 4H), 2.39 (s, 3H), 2.40-2.58 (m, 2H), 2.75-2.88 (m, 2H), 5.07-5.20 (m, 1H), 7.18 (bs, 1H), 7.38-7.53 (m, 3H), 7.65-7.73 (m, 3H), 7.94 (bs, 1H).

### Example 58

¹HNMR (CDCl₃): δ 1.88-2.38 (m, 4H), 2.37 (s, 3H), 2.38-2.56 (m, 2H), 2.73-2.88 (m, 2H), 5.07-5.20 (m, 1H), 7.20 (bs, 1H), 7.33-7.36 (m, 2H), 7.48 (bs, 1H), 7.52-7.54 (m, 1H), 7.81 (bs, 1H).

### Example 59

¹HNMR (CDCl₃): δ 1.80-2.23 (m, 4H), 2.28-2.55 (m, 2H), 2.72-2.90 (m, 2H), 3.62 (s, 2H), 5.08-5.13 (m, 1H), 7.17 (bs, 1H), 7.29-7.38 (m, 7H), 7.50 (bs, 1H), 7.54 (t, 1H, J=1.1 Hz), 7.73 (bs, 1H).

### Example 60

¹HNMR (CDCl₃): δ 3.84 (s, 3H), 5.40 (s, 2H), 6.95 (AA′XX′, 2H, J_{AX}=8.6 Hz, J_{AA′/XX′}=2.5 Hz), 7.21 (bs, 1H), 7.33-7.35 (m, 2H), 7.43 (AA′XX′, 2H, J_{AX}=8.8 Hz, J_{AA′/XX′}=2.4 Hz), 7.49 (bs, 1H), 7.53 (t, 1H, J=1.1 Hz), 7.76 (bs, 1H), 10.61 (bs, 1H).

### Example 61

¹HNMR (CDCl₃): δ 5.56 (s, 2H), 7.27-7.31 (m, 1H), 7.34-7.37 (m, 2H), 7.50-7.56 (m, 2H), 7.66 (AA′XX′, 2H, J_{AX}=9.0 Hz, J_{AA′/XX′}=2.2 Hz), 7.79 (bs, 1H), 8.30 (AA′XX′, 2H, J_{AX}=8.7 Hz, J_{AA′/XX′}=2.1 Hz), 10.19 (bs, 1H).

### Example 62

¹H NMR (CDCl₃): δ 5.43 (s, 2H), 7.04-7.18 (m, 2H), 7.21-7.24 (m, 1H), 7.33-7.36 (m, 2H), 7.44-7.52 (m, 3H), 7.54 (t, 1H, J=1.2 Hz), 7.77 (bs, 1H), 10.48 (bs, 1H).

### Example 63

¹H NMR (CDCl₃): δ 5.43 (s, 2H), 7.22-7.25 (m, 1H), 7.33-7.36 (m, 2H), 7.41 (s, 4H), 7.50 (bs, 1H), 7.54 (t, 1H, J=1.2 Hz), 7.77 (bs, 1H), 10.43 (bs, 1H).

### Example 64

¹H NMR (CDCl₃): δ 5.51 (s, 2H), 7.26-7.29 (m, 1H), 7.33-7.36 (m, 2H), 7.51 (bs, 1H), 7.54 (t, 1H, J=1.3 Hz), 7.58-7.72 (m, 4H), 7.79 (bs, 1H).

### Biological Assays:

The compounds described in the examples 1-64 were evaluated in the following biological assays.

### Determination of Cellular Production of Lactate

Confluent HeLa cervical carcinoma cells (ATCC, Cat. No. CCL-2) in a 96-well plate were treated with the compounds described in Examples 1-64, or with the buffer (prepared in DMEM without phenol red or glutamine, containing a 10% dialyzed FBS, 1% Pen-strep; the final concentration of DMSO in all wells was 1%) for 8 hours at 37° C. in an atmosphere composed of 95% air and 5% CO₂. Wells in duplicate were prepared for each treatment. After the 8 hours of treatment, the medium was collected and centrifuged to remove dead cells. A volume of 100 μL of the supernatant was added to 2 μL of a 50 mM solution of p-clorophenylalanine (CPA, used as internal standard for GC/MS analysis). The samples were concentrated, derivatized using as derivatizing agent MTBSTFA containing a 1% of TBDMCS (Thermo Scientific), and finally analyzed by GC/MS (Agilent 6890N GC/5973 MS equipped with a capillary column Agilent DB-5, 30M×320 μM×0.25 μM). The compounds were identified by using databases and softwares, as for example AMDIS (“Automated Mass Spectral Deconvolution and Identification System”). The integration area of lactate obtained with each sample was divided by the integration area of CPA in the same sample to obtain the ratios of the lactate/internal standard. The average values of these ratios were obtained from experiments performed in duplicate and the percentage (%) of lactate production compared to the control samples not treated were calculated for each independent experiment by calculation of the lactate production ratios between treated and control samples. At this point, the average values representing the average percentage of lactate production compared to the control samples were obtained from experiments performed in triplicate.

Examples 1, 3, 8, 9, 22, 23, 31, 32, 33, 40 and 41, are able to reduce effectively cellular production of lactate in HeLa cells treated with concentrations ranging from 50 to 200 μM, in a manner comparable or superior to the treatment with 2-DeoxGlu at a concentration of 10 mM.

### Assessment of Inhibition of Tumor Cell Growth Method (a)

Confluent cells obtained from ATCC (American Type Culture Collection) of cervical carcinoma HeLa (ATCC, Cat. No. CCL-2), breast carcinoma MCF-7 (ATCC, Cat. No. HTB-22), non-small cell lung carcinoma (NSCLC), H1299 (ATCC, Cat. No. CRL-5803) and H226 (ATCC, Cat. No. CRL-5826), and ovarian cancer IGROV-1 [Bénard, J. et al. Cancer Res. 1985 45, 4970-4979], were grown in culture medium RPMI (Roswell Park-Memorial-Institute) 1640 supplemented with 10% FBS and with a 1% Pen-strep, and were added in 96-well plates, to a density of 5000 cells per well. Solutions of the compounds were added in DMSO at final concentrations ranging from 31.6 nM-200 μM (final DMSO concentration of 1% in all wells; each experiment was repeated in triplicate for each concentration). The plates were incubated at 37° C. in an atmosphere composed of 95% air and 5% CO₂ for 72 hours. The culture medium was then removed and the cells were fixed by addition of 50 μL of a 10% solution of trichloroacetic acid in water at 4° C. in each well. The plates were incubated at 4° C. for at least one hour, after which the colorimetric assay of sulforhodamine B (SRB) was carried out to determine the amount of biomass remaining in each well as described in previously developed methodologies [Vichai, V.; Kirtikara, K. Nat. Protoc. 2006, 1, 1112-6]. Briefly, the plates were washed several times with water and dried prior to the addition of 50 μL of a solution of dye sulforhodamine B (composed by 0.057% weight/weight of sulforhodamine B in 1% acetic acid) to each well. After 30 minutes of incubation, the unbound dye was removed by washing six times with 1% acetic acid. Subsequently, 200 microliters of 10 mM Tris buffer (pH 10.5) were added to each dried well, in order to re-solubilize the dye bound to the biomass. After an incubation period of 30 minutes, the absorbance in each well was read at a wavelength of 510 nm in a microplate reader. The cells treated only with vehicle consisting of a 1% solution of DMSO (vehicle) were used as control of 100% of live cells in the biomass and the wells incubated with the vehicle alone (without cells) were used to determine the baseline (0%) of live biomass. The IC₅₀ values were calculated using the software SoftMax Pro (Molecular Devices, Sunnyvale, Calif.).

The following Table 1 reports the experimental data (IC₅₀ μM) obtained testing some representative compounds of the formula (I) of the invention, identified with the number used above, in the above described proliferation assays, in comparison with a prior art compound, described in the aforementioned WO 2011/054525, (example 20, page 46), chemical name 1-hydroxy-6-phenyl-4-trifluoromethyl-1H-indol-2-carboxylic acid, and coded therein as Example 20.

TABLE 1

| | cell | IC₅₀ value |
|---|---|---|
| compound | line | (μM) |
| 1-hydroxy-6-phenyl-4-trifluoromethyl-1H- | Hela | 44 |
| indol-2-carboxylic acid, comprised in WO | MCF-7 | 124 |
| 2011/054525 (example 20, page 46) | H1299 | 141 |
| | H226 | 121 |
| | IGROV-1 | 123 |
| representative examples 1 and 3 the | Hela | <17 |
| present invention | MCF-7 | <35 |
| | H1299 | <40 |
| | H226 | <26 |
| | IGROV-1 | <31 |
| representative examples 8 and 10 of the | Hela | <15 |
| present invention | MCF-7 | <17 |

### Assessment of Inhibition of Tumor Cell Growth Method (b)

CellTiter-Glo® Luminescent Cell Viability Assay (Promega) is a homogeneous method of determining the number of viable cells in culture based on quantitation of the present ATP, which indicates the presence of metabolically active cells. The homogeneous assay procedure involves addition of a single reagent (CellTiter-Glo® Reagent) directly to the cells, which leads to cell lysis and generation of a luminescent signal proportional to the amount of the ATP and the number of cells present in culture. The assay relies on the properties of a proprietary thermostable luciferase (Ultra-Glo® recombinant luciferase), which generates a luminescent signal.

Human cancer cells (A549 cells from Adenocarcinomic alveolar basal epithelial (ATCC, Cat. No. CCL-185) and H1975 non small cells from adenocarcinoma (ATCC, Cat. No. CRL-5908)), in exponential growth, were incubated for 72 h with different concentrations of the inhibitors. After 72 h, a volume of CellTiter-Glo® Reagent equal to the volume of cell culture medium was added. The content was mixed for 2 min to induce cell lysis. The luminescence was recorded after further 10 min at RT in order to obtain a stable luminescent signal.

The IC₅₀ was calculated using GraphPad Software.

The following Table 2 reports the experimental data (IC₅₀ μM) obtained testing some representative compounds of the formla (I) of the invention, identified with the number used above, in the above described proliferation assays, in comparison with a prior art compound, described in the aforementioned WO 2011/054525, (example 20, page 46), chemical name 1-hydroxy-6-phenyl-4-trifluoromethyl-1H-indol-2-carboxylic acid, and coded therein as Example 20.

TABLE 2

| | cell | IC₅₀ value |
|---|---|---|
| compound | line | (uM) |
| 1-hydroxy-6-phenyl-4-trifluoromethyl-1H- | A549 | 60 |
| indol-2-carboxylic acid, comprised in WO | H1975 | 57 |
| 2011/054525 (example 20, page 46) | | |
| representative examples of the present | A549 | ≦31 |
| invention: 1, 3, 8, 9, 19, 22, 23, 25, 32, | | |
| 34, 35, 36, 38, 42, 43, 48, and 53 | | |
| representative examples of the present | H1975 | ≦31 |
| invention: 1, 3, 8, 9, 19, 22, 23, 32, 34, | | |
| 35, 36, 38, 42, 48, and 53 | | |

### Determination of Enzyme Inhibition Parameters of Isoform 5 (LDH5, LDH-A) and 1 (LDH1, LDH-B) of Human Lactate Dehydrogenase.

The compounds described in the examples were evaluated in enzymatic assays to assess its inhibitory properties against two human isoforms of lactate dehydrogenase, hLDH5 containing solely the subunit LDH-A (LEEBIO—USA), and the hLDH1 containing only the LDH subunits-B (Sigma Aldrich, USA), in order to verify the selectivity of these compounds.

The reaction of lactate dehydrogenase was conducted using the “forward” direction (pyruvate→lactate) and the kinetic parameters for the substrate (pyruvate) and the cofactor (NADH) were measured by spectrophotometric absorbance at a wavelength of 340 nm, or by fluorescence (emission at 460 nm, excitation at 340 nm), to monitor, at room temperature, the amount of NADH consumed (for IC₅₀ measurements), or the rate of conversion of NADH to NAD⁺ and, therefore, the progression of the reaction at 37° C. (for Kᵢ measurements). Such assays were conducted in cells containing 200 μL of a solution comprising the reagents dissolved in phosphate buffer (KH₂PO₄ and K₂HPO₄) at pH 7.4.

IC₅₀ values were calculated as described below. DMSO stock solution of compounds were prepared (concentration of DMSO did not exceed 5% during the measurements). Seven different concentrations (in duplicate for each concentration) of compound were used to generate a concentration-response curve. In the NADH-competition assay, compounds were tested in the presence of 40 μM NADH and 1440 μM pyruvate; in pyruvate-competition assay, the concentrations of NADH and pyruvate were 150 and 200 μM, respectively. Compound solutions were dispensed in 96-well plates (8 μL), then substrate and cofactor dissolved in buffer (152 μl) and enzyme solution (40 μl) were finally added. Dilution of the enzyme stock solution was made to allow a 10% consumption of the cofactor after 15 min. The eventual background fluorescence of the tested compounds or quenching of the NADH fluorescence by the tested compounds was subtracted. In addition to the compound test wells, each plate contained maximum and minimum controls. Assay plates were incubated at room temperature for 15 min and the final measurements were performed using Victor X3 Microplates reader (PerkinElmer®) at a fluorescence emission wavelength of 460 nm (excitation at 340 nm). IC₅₀ were generated using the curve-fitting tool of GraphPad Prism].

The kinetic parameters for the isoform hLDH1 in respect to the pyruvate were calculated by measuring the initial rate of the reaction with the pyruvate concentrations ranging between 40 and 504 μM and NADH at 150 μM. Then, the kinetic parameters for the same isoform, but in respect to NADH, were calculated by measuring the initial rate of the reaction with concentrations of NADH ranging between 9.6 μM and 60 μM and pyruvate at 1.4 mM.

The kinetic parameters for the isoform hLDH5 in respect to the pyruvate were calculated by measuring the initial rate of the reaction at concentrations of pyruvate ranging between 40 and 504 μM and NADH at 150 μM. Then, the kinetic parameters for the same isoform, but in respect to NADH, were calculated by measuring the initial rate of the reaction using at concentrations of NADH ranging between 9.6 μM and 60 μM and pyruvate at 1.4 mM.

The resulting data of enzymatic kinetic (the constants of Michaelis-Menten) were determined by analysis of non-linear regression. The Kᵢ values for each active compound were obtained using a Lineweaver-Burk plot or a second order polynomial regression analysis, by applying the mixed-model inhibition fit.

The compounds reported in the Examples 1-64 show one or more of the following:
inhibitory activity against the production of lactic acid by tumoral cells, for example, but not limited to the case of, HeLa cells, with cellular production of lactic acid reduced to a range between 2% and 50% compared the untreated cells (control), upon treatment with concentrations ranging between 50 and 200 μM of compound;
inhibitory activity against the isoform hLDH5 in competition experiments vs. cofactor NADH with Ki values ranging between 0.01 and 10000 μM;
inhibitory activity against the isoform hLDH5 in competition experiments vs. the substrate pyruvate with Ki values ranging between 0.01 and 10000 μM;
inhibitory activity against the isoforma hLDH1 in competition experiments vs. the cofactor NADH with Ki values ranging between 0.01 and 10000 μM.

The following examples show an inhibitory activity against hLDH5 expressed as either IC₅₀ or Kᵢ displaying the following ranges of values: Examples 33, 49, 50, 55, 56, 60, 61, 62, 63, and 64 within 0.01-1.0 μM (IC₅₀); Examples 22, 24, 32, and 48 within 0.50-5.0 μM (Kᵢ); Examples 1, 8, 9, 18, 19, 23, 25, 27, 36, 37, 42, and 43 within 1.0-10 μM (Kᵢ); Examples 14-17, 20, and 26 within 1.0-10 μM (IC₅₀); Examples 34, 35, 38, 39, and 52-54 within 5.0-25 μM (Kᵢ); Examples 2, 3, 40, 41, and 51 within 10-100 μM (Kᵢ); Examples 5, 6, 10, 12, and 31 within 50-500 μM (IC₅₀); Examples 4, 7, 11, 13, 21, 28, 44-47 and 57-59>100 μM (IC₅₀).

### DESCRIPTION OF A PREFERRED EMBODIMENT

As example, HeLa cells of cervical cancer were incubated for 8 hours in the presence of varying concentrations (50-200 μM) of compounds of the present invention. Then, the amount of lactic acid produced by these cells was determined by derivatization of lactic acid with N-methyl-N-(tert-butyldimethylsilyl)trifluoroacetamide (MTBSTFA) in presence of 1% of tert-butyldimethylclorosilane (TBDMCS) and analysis by gas-chromatography, using as internal standard the L-(p-chlorophenyl) alanine.

The basal production of lactic acid was determined by incubating cells with the vehicle (0.2% DMSO in buffer) alone and was normalized to 100%. As references, we used two known inhibitors of the hexokinase, such as 2-deoxyglucose (2-DeoxGlu) [Bachelard, H. S., Clark, A. G., Thompson, M. F., Biochem. J. 1971, 123, 707-715] and the 3-bromopyruvate (3-BrPyr) [Kim, W. et al. Mol. Cancer. Ther. 2007, 6, 2554-2562]. The compound n-FLY-21 was used as reference [WO2011054525].

Some representative examples of the present invention, such as examples 1, 3, 8, 9, 22, 23, 31, 32, 33, 40 and 41, are able to reduce effectively cellular production of lactate in HeLa cells treated with concentrations ranging from 50 to 200 μM, in a manner comparable or superior to the treatment with 2-DeoxGlu at a concentration of 10 mM. Furthermore, some representative examples showed cytotoxic activity against some selected tumor cell lines, as HeLa (cervix), A549 (lung), MCF-7 (breast), H1299 (lung), H226 (lung) IGROV-1 (ovarian) and H1975 (lung) cells.

1. A compound, having the general formula (I): 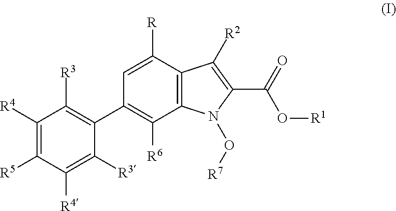
wherein:
R is F or CF₃;
R¹ is H; C₁-C₄ alkyl; C₁-C₄ alkyl substituted by phenyl, wherein the phenyl may optionally be substituted with one or more groups selected from halogen, nitro, methoxy, CF₃ or phenyl; C₁-C₄ alkyl substituted by C₃-C₇ cycloalkyl, wherein the C₃-C₇ cycloalkyl may optionally be substituted by C₁-C₄ alkyl; or piperidine, optionally substituted by C₁-C₄ alkyl or C₁-C₄ alkyl substituted by phenyl;
R² is H or CH₃;
R³, R⁴, R³′, R⁴′ and R⁵ are independently selected from H, Cl, or OCF₃;
R⁶ is H or C₆H₅;
R⁷ is H, 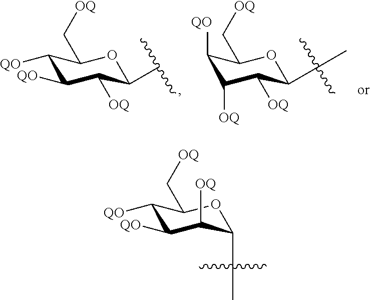
wherein Q is selected from H or CH₃C(O); or a stereoisomer, tautomer, hydrate, solvate, or a pharmaceutically acceptable salt thereof;
with the exclusion of the following compounds;
wherein R═CF₃ and:
R¹, R², R³, R⁴, R³′, R⁴′, R⁵, R⁶, and R⁷═H;
R¹, R², R³, R⁴, R³′, R⁴′, R⁵, and R⁷═H; R⁶═C₆H₅;
R¹, R², R³, R⁴, R³′, R⁴′, R⁶, and R⁷═H; R⁵═Cl;
R¹, R³, R⁴, R³′, R⁴′, R⁵, R⁶ and R⁷═H; R²═CH₃;
R¹, R³, R⁴, R³′, R⁴′, R⁶, and R⁷═H; R²═CH₃; R⁵═Cl; and
R¹, R², R³′, R⁴′, R⁴, R⁶, and R⁷═H; R³, R⁵═Cl.

2. The compound according to claim 1, wherein R═CF₃.

3. The compound according to claim 1, wherein R═F.

4. The compound according to claim 1,
wherein R¹ is independently H, CH₃, CH₂CH₃, CH(CH₃)₂, (CH₂)₃CH₃ or CH₂(C₆H₅) or methyl substituted by a phenyl,
wherein the phenyl may be unsubstituted or substituted by one or more groups selected from halogen, nitro, methoxy, CF₃ or phenyl; or piperidine N-substituted by CH₃ or CH₂(C₆H₅); or 4-(tert-butyl)cyclohexyl.

5. The compound according to claim 1, wherein R⁷ is H, R⁵ is Cl and R⁴, R⁴′ are independently H or Cl.

6. The compound according to claim 1,
wherein R⁷ is H and
R³, R⁴, R³′, R⁴′ are independently H or Cl.

7. The compound according to claim 1,
wherein R⁷ is H and
R³, R⁴, R³′, R⁴′, R⁵ are independently H or OCF₃.

8. The compound according to claim 1,
wherein R⁷ is

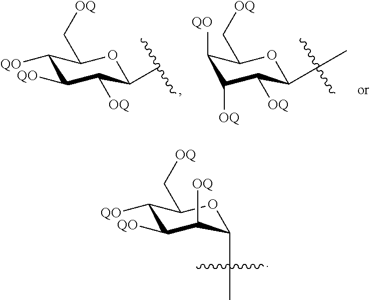

9. The compound according to claim 1,
wherein at least one of R¹ and R⁷ is from hydrogen.

10. A compound of formula (I) selected from the group consisting of:
ethyl 1-hydroxy-6-phenyl-4-(trifluoromethyl)-1H-indole-2-carboxylate (Example 1);
6-phenyl-1-(β-D-glucopyranosyl)oxy-4-(trifluoromethyl)-1H-indole-2-carboxylic acid (Example 2);
methyl 6-phenyl-1-(β-D-glucopyranosyl)oxy-4-(trifluoromethyl)-1H-indole-2-carboxylate (Example 3);
6-phenyl-1-(β-D-2,3,4,6-tetra-O-acetylglucopyranosyl)oxy-4-(trifluoromethyl)-1H-indole-2-carboxylic acid (Example 4);
methyl 6-phenyl-1-(β-D-2,3,4,6-tetra-O-acetylglucopyranosyl)oxy-4-(trifluoromethyl)-1H-indole-2-carboxylate (Example 5);
ethyl 6-phenyl-1-(β-D-glucopyranosyl)oxy-4-(trifluoromethyl)-1H-indole-2-carboxylate (Example 6);
ethyl 6-phenyl-1-(β-D-2,3,4,6-tetra-O-acetylglucopyranosyl)oxy-4-(trifluoromethyl)-1H-indole-2-carboxylate (Example 7);
methyl 6-(2,4-dichlorophenyl)-1-hydroxy-4-(trifluoromethyl)-1H-indole-2-carboxylate (Example 8);
ethyl 6-(2,4-dichlorophenyl)-1-hydroxy-4-(trifluoromethyl)-1H-indole-2-carboxylate (Example 9);
methyl 6-(2,4-dichlorophenyl)-1-(β-D-glucopyranosyl)oxy-4-(trifluoromethyl)-1H-indole-2-carboxylate (Example 10);
methyl 6-(2,4-dichlorophenyl)-1-(β-D-2,3,4,6-tetra-O-acetylglucopyranosyl)oxy-4-(trifluoromethyl)-1H-indole-2-carboxylate (Example 11);
ethyl 6-(2,4-dichlorophenyl)-1-(β-D-glucopyranosyl)oxy-4-(trifluoromethyl)-1H-indole-2-carboxylate (Example 12);
ethyl 6-(2,4-dichlorophenyl)-1-(β-D-2,3,4,6-tetra-O-acetylglucopyranosyl)oxy-4-(trifluoromethyl)-1H-indole-2-carboxylate (Example 13);
methyl 1-idroxy-6,7-diphenyl-4-(trifluoromethyl)-1H-indole-2-carboxylate (Example 14);
methyl 6-(4-clorophenyl)-1-hydroxy-4-(trifluoromethyl)-1H-indole-2-carboxylate (Example 15);
methyl 1-hydroxy-6-phenyl-3-methyl-4-(trifluoromethyl)-1H-indole-2-carboxylate (Example 16);
methyl 1-hydroxy-6-(4-clorophenyl)-3-methyl-4-(trifluoromethyl)-1H-indole-2-carboxylate (Example 17);
methyl 1-hydroxy-4-(trifluoromethyl)-6-(4-(trifluoromethoxy)phenyl)-1H-indole-2-carboxylate (Example 18);
methyl 1-hydroxy-4-(trifluoromethyl)-6-(3-(trifluoromethoxy)phenyl)-1H-indole-2-carboxylate (Example 19);
methyl 6-(2,4-dichlorophenyl)-1-hydroxy-3-methyl-4-(trifluoromethyl)-1H-indole-2-carboxylate (Example 20);
methyl 6-(3,4-dichlorophenyl)-1-hydroxy-4-(trifluoromethyl)-1H-indole-2-carboxylate (Example 21);
butyl 1-hydroxy-6-phenyl-4-(trifluoromethyl)-1H-indole-2-carboxylate (Example 22);
isopropyle isopropyl 1-hydroxy-6-phenyl-4-(trifluoromethyl)-1H-indole-2-carboxylate (Example 23);
1-hydroxy-4-(trifluoromethyl)-6-(4-(trifluoromethoxy)phenyl)-1H-indole-2-carboxylic acid (Example 24);
1-hydroxy-4-(trifluoromethyl)-6-(3-(trifluoromethoxy)phenyl)-1H-indole-2-carboxylic acid (Example 25);
6-(2,4-dichlorophenyl)-1-hydroxy-3-methyl-4-(trifluoromethyl)-1H-indole-2-carboxilic acid (Example 26);
6-(3,4-dichlorophenyl)-1-hydroxy-4-(trifluoromethyl)-1H-indole-2-carboxilic acid (Example 27);
butyl 6-phenyl-1-(β-D-2,3,4,6-tetra-O-acetylglucopyranosyl)oxy-4-(trifluoromethyl)-1H-indole-2-carboxylate (Example 28);
butyl 1-(β-D-glucopyranosyl)oxy-6-phenyl-4-(trifluoromethyl)-1H-indole-2-carboxylate (Example 29);
isopropyl 6-phenyl-1-(β-D-2,3,4,6-tetra-O-acetylglucopyranosyl)oxy-4-(trifluoromethyl)-1H-indole-2-carboxylate (Example 30);
isopropyl 1-(β-D-glucopyranosyl)oxy-6-phenyl-4-(trifluoromethyl)-1H-indole-2-carboxylate (Example 31);
isopropyl 6-(2,4-dichlorophenyl)-1-hydroxy-4-(trifluoromethyl)-1H-indole-2-carboxylate (Example 32);
butyl 6-(2,4-dichlorophenyl)-1-hydroxy-4-(trifluoromethyl)-1H-indole-2-carboxylate (Example 33);
methyl 1-hydroxy-6-(2-(trifluoromethoxy)phenyl)-4-(trifluoromethyl)-1H-indole-2-carboxylate (Example 34);
1-hydroxy-6-(2-(trifluoromethoxy)phenyl)-4-(trifluoromethyl)-1H-indole-2-carboxylic acid (Example 35);
methyl 6-(2,3-dichlorophenyl)-1-hydroxy-4-(trifluoromethyl)-1H-indole-2-carboxylate (Example 36);
6-(2,3-dichlorophenyl)-1-hydroxy-4-(trifluoromethyl)-1H-indole-2-carboxylic acid (Example 37);
methyl 6-(2,5-dichlorophenyl)-1-hydroxy-4-(trifluoromethyl)-1H-indole-2-carboxylate (Example 38);
6-(2,5-dichlorophenyl)-1-hydroxy-4-(trifluoromethyl)-1H-indole-2-carboxylic acid (Example 39);
methyl 1-(β-D-gulopyranosyl)oxy-6-phenyl-4-(trifluoromethyl)-1H-indole-2-carboxylate (Example 40);
methyl 1-(α-D-mannopyranosyl)oxy-6-phenyl-4-(trifluoromethyl)-1H-indole-2-carboxylate (Example 41);
methyl 6-(3,5-dichlorophenyl)-1-hydroxy-4-(trifluoromethyl)-1H-indole-2-carboxylate (Example 42);
6-(3,5-dichlorophenyl)-1-hydroxy-4-(trifluoromethyl)-1H-indole-2-carboxylic acid (Example 43);
isopropyl 6-(2,4-dichlorophenyl)-1-(β-D-2,3,4,6-tetra-O-acetylglucopyranosyl)oxy-4-(trifluoromethyl)-1H-indole-2-carboxylate (Example 44);
isopropyl 6-(2,4-dichlorophenyl)-1-(β-D-glucopyranosyl)oxy-4-(trifluoromethyl)-1H-indole-2-carboxylate (Example 45);
butyl 6-(2,4-dichlorophenyl)-1-(β-D-2,3,4,6-tetra-O-acetylglucopyranosyl)oxy-4-(trifluoromethyl)-1H-indole-2-carboxylate (Example 46);
butyl 6-(2,4-dichlorophenyl)-1-(β-D-glucopyranosyl)oxy-4-(trifluoromethyl)-1H-indole-2-carboxylate (Example 47);
benzyl 1-hydroxy-6-phenyl-4-(trifluoromethyl)-1H-indole-2-carboxylate (Example 48);
4-(tert-butyl)cyclohexyl 1-hydroxy-6-phenyl-4-(trifluoromethyl)-1H-indole-2-carboxylate (Example 49);
4-(tert-butyl)cyclohexyl 6-(2,4-dichlorophenyl)-1-hydroxy-4-(trifluoromethyl)-1H-indole-2-carboxylate (Example 50);
methyl 4-fluoro-1-hydroxy-6-phenyl-1H-indole-2-carboxylate (Example 51);
4-fluoro-1-hydroxy-6-phenyl-1H-indole-2-carboxylic acid (Example 52);
methyl 6-(2,4-dichlorophenyl)-4-fluoro-1-hydroxy-1H-indole-2-carboxylate (Example 53);
6-(2,4-dichlorophenyl)-4-fluoro-1-hydroxy-1H-indole-2-carboxylic acid (Example 54);
benzyl 6-(2,4-dichlorophenyl)-1-hydroxy-4-(trifluoromethyl)-1H-indole-2-carboxylate (Example 55);
[1,1′-biphenyl]-4-ylmethyl 6-(2,4-dichlorophenyl)-1-hydroxy-4-(trifluoromethyl)-1H-indole-2-carboxylate (Example 56);
1-methylpiperidin-4-yl 1-hydroxy-6-phenyl-4-(trifluoromethyl)-1H-indole-2-carboxylate (Example 57);
1-methylpiperidin-4-yl 6-(2,4-dichlorophenyl)-1-hydroxy-4-(trifluoromethyl)-1H-indole-2-carboxylate (Example 58);
1-benzylpiperidin-4-yl 6-(2,4-dichlorophenyl)-1-hydroxy-4-(trifluoromethyl)-1H-indole-2-carboxylate (Example 59);
4-methoxybenzyl 6-(2,4-dichlorophenyl)-1-hydroxy-4-(trifluoromethyl)-1H-indole-2-carboxylate (Example 60);
4-nitrobenzyl 6-(2,4-dichlorophenyl)-1-hydroxy-4-(trifluoromethyl)-1H-indole-2-carboxylate (Example 61);
4-fluorobenzyl 6-(2,4-dichlorophenyl)-1-hydroxy-4-(trifluoromethyl)-1H-indole-2-carboxylate (Example 62);
4-chlorobenzyl 6-(2,4-dichlorophenyl)-1-hydroxy-4-(trifluoromethyl)-1H-indole-2-carboxylate (Example 63); and
4-(trifluoromethyl)benzyl 6-(2,4-dichlorophenyl)-1-hydroxy-4-(trifluoromethyl)-1H-indole-2-carboxylate (Example 64).

11. A method for the treatment of cancer, comprising administering a compound of claim 1 to a patient in need thereof.

12. The method of claim 11, wherein the cancer is selected from the group consisting of:
lymphoma;
hepatocellular carcinoma;
pancreatic cancer;
brain tumor;
breast cancer;
lung cancer;
colon cancer;
cervical cancer;
prostate cancer;
kidney cancer;
osteosarcoma;
nasopharyngeal cancer;
oral cavity cancer;
melanoma; and
ovarian cancer.

13. The method according to claim 12, wherein the cancer is selected from the group consisting of:
lung tumor;
breast cancer;
cervical cancer; and
ovarian cancer.

14. The method according to claim 13, wherein the lung cancer is a non small cell lung carcinoma.

15. A method of treating a disease selected from the group consisting of asthma, pulmonary hypertension, idiopathic arthrofibrosis, malaria, chronic back, or of hyperoxaluria, comprising administering a compound of claim 1 to a patient in need thereof.

16. A pharmaceutical composition comprising at least one compound as defined in claim 1 or a stereoisomer, tautomer, hydrate, solvate, or pharmaceutically acceptable salt thereof and at least one pharmaceutically acceptable excipient and/or diluent.

17. A compound of general formula (I) 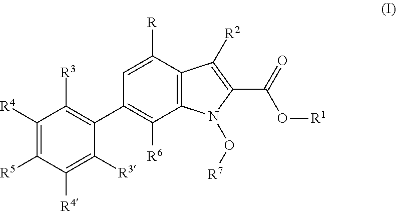
or a stereoisomer, tautomer, hydrate, solvate or a pharmaceutically acceptable salt of said compound,
wherein:
R is F or CF₃;
R¹ is H; C₁-C₄ alkyl; C₁-C₄ alkyl substituted by a phenyl, wherein the phenyl may be optionally substituted by one or more groups selected from halogen, nitro, methoxy, CF₃ or phenyl; C₁-C₄ alkyl substituted by C₃-C₇ cycloalkyl, wherein the C₃-C₇ cycloalkyl is optionally substituted by C₁-C₄ alkyl; or piperidine, optionally substituted by C₁-C₄ alkyl or C₁-C₄ alkyl substituted by phenyl;
R² is H or CH₃;
R³, R⁴, R³′, R⁴′ and R⁵ are independently H, Cl, or OCF₃;
R⁶ is H or C₆H₅;
R⁷ is H, 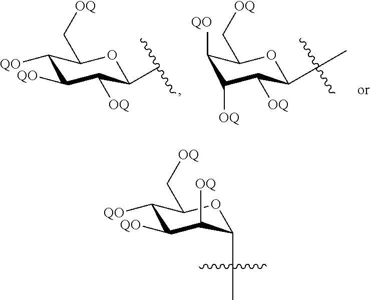
wherein Q is H or CH₃C(O);
with the exclusion of the following compounds;
wherein R═CF₃ and
R¹, R², R³, R⁴, R³′, R⁴′, R⁵, R⁶ and R⁷═H;
R¹, R², R³, R⁴, R³′, R⁴′, R⁵, and R⁷═H; R⁶═C₆H₅;
R¹, R², R³, R⁴, R³′, R⁴′, R⁶, and R⁷═H; R⁵═Cl;
R¹, R³, R⁴, R³′, R⁴′, R⁵, R⁶ and R⁷═H; R²═CH₃;
R¹, R³, R⁴, R³′, R⁴′, R⁶, and R⁷═H; R²═CH₃; R⁵═Cl;
R¹, R², R⁴, R³′, R⁴′, R⁶, and R⁷═H; R³, R⁵═Cl;
R¹═CH₃; R², R³, R⁴, R³′, R⁴′, R⁵, R⁶ and R⁷═H;
R′═CH₃; R², R³, R⁴, R³′, R⁴′, R⁵, and R⁷═H; R⁶═C₆H₅;
R¹═CH₃; R², R³, R⁴, R³′, R⁴′, R⁶, and R⁷═H; R⁵═Cl;
R¹═CH₃; R³, R⁴, R³′, R⁴′, R⁵, R⁶ and R⁷═H; R²═CH₃;
R¹═CH₃; R³, R⁴, R³′, R⁴′, R⁶, and R⁷═H; R²═CH₃; R⁵═Cl; and
R¹═CH₃; R², R⁴, R³′, R⁴′, R⁶, and R⁷═H; R³, R⁵═Cl.

18. The compound according to claim 17 wherein at least one of R¹ and R⁷ is not hydrogen.

19. The compound according to claim 17 selected from the group consisting of:
ethyl 1-hydroxy-6-phenyl-4-(trifluoromethyl)-1H-indole-2-carboxylate (Example 1);
6-phenyl-1-(β-D-glucopyranosyl)oxy-4-(trifluoromethyl)-1H-indole-2-carboxylic acid (Example 2);
methyl 6-phenyl-1-(β-D-glucopyranosyl)oxy-4-(trifluoromethyl)-1H-indole-2-carboxylate (Example 3);
6-phenyl-1-(β-D-2,3,4,6-tetra-O-acetylglucopyranosyl)oxy-4-(trifluoromethyl)-1H-indole-2-carboxylic acid (Example 4);
methyl 6-phenyl-1-(β-D-2,3,4,6-tetra-O-acetylglucopyranosyl)oxy-4-(trifluoromethyl)-1H-indole-2-carboxylate (Example 5);
ethyl 6-phenyl-1-(β-D-glucopyranosyl)oxy-4-(trifluoromethyl)-1H-indole-2-carboxylate (Example 6);
ethyl 6-phenyl-1-(β-D-2,3,4,6-tetra-O-acetylglucopyranosyl)oxy-4-(trifluoromethyl)-1H-indole-2-carboxylate (Example 7);
ethyl 6-(2,4-dichlorophenyl)-1-hydroxy-4-(trifluoromethyl)-1H-indole-2-carboxylate (Example 9);
methyl 6-(2,4-dichlorophenyl)-1-(β-D-glucopyranosyl)oxy-4-(trifluoromethyl)-1H-indole-2-carboxylate (Example 10);
methyl 6-(2,4-dichlorophenyl)-1-(β-D-2,3,4,6-tetra-O-acetylglucopyranosyl)oxy-4-(trifluoromethyl)-1H-indole-2-carboxylate (Example 11);
ethyl 6-(2,4-dichlorophenyl)-1-(β-D-glucopyranosyl)oxy-4-(trifluoromethyl)-1H-indole-2-carboxylate (Example 12);
ethyl 6-(2,4-dichlorophenyl)-1-(β-D-2,3,4,6-tetra-O-acetylglucopyranosyl)oxy-4-(trifluoromethyl)-1H-indole-2-carboxylate (Example 13);
methyl 1-idroxy-4-(trifluoromethyl)-6-(4-(trifluoromethoxy)phenyl)-1H-indole-2-carboxylate (Example 18);
methyl 1-idroxy-4-(trifluoromethyl)-6-(3-(trifluoromethoxy)phenyl)-1H-indole-2-carboxylate (Example 19);
methyl 6-(2,4-dichlorophenyl)-1-hydroxy-3-methyl-4-(trifluoromethyl)-1H-indole-2-carboxylate (Example 20);
methyl 6-(3,4-dichlorophenyl)-1-hydroxy-4-(trifluoromethyl)-1H-indole-2-carboxylate (Example 21);
butyl 1-hydroxy-6-phenyl-4-(trifluoromethyl)-1H-indole-2-carboxylate (Example 22);
isopropyl 1-hydroxy-6-phenyl-4-(trifluoromethyl)-1H-indole-2-carboxylate (Example 23);
1-hydroxy-4-(trifluoromethyl)-6-(4-(trifluoromethoxy)phenyl)-1H-indole-2-carboxylic acid (Example 24);
1-hydroxy-4-(trifluoromethyl)-6-(3-(trifluoromethoxy)phenyl)-1H-indole-2-carboxylic acid (Example 25);
6-(2,4-dichlorophenyl)-1-hydroxy-3-methyl-4-(trifluoromethyl)-1H-indole-2-carboxylic acid (Example 26);
6-(3,4-dichlorophenyl)-1-hydroxy-4-(trifluoromethyl)-1H-indole-2-carboxylic acid (Example 27);
butyl 6-phenyl-1-(β-D-2,3,4,6-tetra-O-acetylglucopyranosyl)oxy-4-(trifluoromethyl)-1H-indole-2-carboxylate (Example 28);
butyl 1-(β-D-glucopyranosyl)oxy-6-phenyl-4-(trifluoromethyl)-1H-indole-2-carboxylate (Example 29);
butyl 1-(β-D-2,3,4,6-tetra-O-acetylglucopyranosyl)oxy-4-(trifluoromethyl)-1H-indole-2-carboxylate (Example 30);
isopropyl 1-(β-D-glucopyranosyl)oxy-6-phenyl-4-(trifluoromethyl)-1H-indole-2-carboxylate (Example 31);
isopropyl 6-(2,4-dichlorophenyl)-1-hydroxy-4-(trifluoromethyl)-1H-indole-2-carboxylate (Example 32);
butyl 6-(2,4-dichlorophenyl)-1-hydroxy-4-(trifluoromethyl)-1H-indole-2-carboxylate (Example 33);
methyl 1-hydroxy-6-(2-(trifluoromethoxy)phenyl)-4-(trifluoromethyl)-1H-indole-2-carboxylate (Example 34);
1-hydroxy-6-(2-(trifluoromethoxy)phenyl)-4-(trifluoromethyl)-1H-indole-2-carboxylic acid (Example 35);
methyl 6-(2,3-dichlorophenyl)-1-hydroxy-4-(trifluoromethyl)-1H-indole-2-carboxylate (Example 36);
6-(2,3-dichlorophenyl)-1-hydroxy-4-(trifluoromethyl)-1H-indole-2-carboxylic acid (Example 37);
methyl 6-(2,5-dichlorophenyl)-1-hydroxy-4-(trifluoromethyl)-1H-indole-2-carboxylate (Example 38);
6-(2,5-dichlorophenyl)-1-hydroxy-4-(trifluoromethyl)-1H-indole-2-carboxylic acid (Example 39);
methyl 1-(β-D-gulopyranosyl)oxy-6-phenyl-4-(trifluoromethyl)-1H-indole-2-carboxylate (Example 40);
methyl 1-(α-D-mannopyranosyl)oxy-6-phenyl-4-(trifluoromethyl)-1H-indole-2-carboxylate (Example 41);
methyl 6-(3,5-dichlorophenyl)-1-hydroxy-4-(trifluoromethyl)-1H-indole-2-carboxylate (Example 42);
6-(3,5-dichlorophenyl)-1-hydroxy-4-(trifluoromethyl)-1H-indole-2-carboxylic acid (Example 43);
isopropyl 6-(2,4-dichlorophenyl)-1-(β-D-2,3,4,6-tetra-O-acetylglucopyranosyl)oxy-4-(trifluoromethyl)-1H-indole-2-carboxylate (Example 44);
isopropyl 6-(2,4-dichlorophenyl)-1-(β-D-glucopyranosyl)oxy-4-(trifluoromethyl)-1H-indole-2-carboxylate (Example 45);
butyl 6-(2,4-dichlorophenyl)-1-(β-D-2,3,4,6-tetra-O-acetylglucopyranosyl)oxy-4-(trifluoromethyl)-1H-indole-2-carboxylate (Example 46);
butyl 6-(2,4-dichlorophenyl)-1-(β-D-glucopyranosyl)oxy-4-(trifluoromethyl)-1H-indole-2-carboxylate (Example 47);
benzyl 1-hydroxy-6-phenyl-4-(trifluoromethyl)-1H-indole-2-carboxylate (Example 48);
4-(tert-butyl)cyclohexyl 1-hydroxy-6-phenyl-4-(trifluoromethyl)-1H-indole-2-carboxylate (Example 49);
4-(tert-butyl)cyclohexyl 6-(2,4-dichlorophenyl)-1-hydroxy-4-(trifluoromethyl)-1H-indole-2-carboxylate (Example 50);
methyl 4-fluoro-1-hydroxy-6-phenyl-1H-indole-2-carboxylate (Example 51);
4-fluoro-1-hydroxy-6-phenyl-1H-indole-2-carboxylic acid (Example 52);
methyl 6-(2,4-dichlorophenyl)-4-fluoro-1-hydroxy-1H-indole-2-carboxylate (Example 53);
6-(2,4-dichlorophenyl)-4-fluoro-1-hydroxy-1H-indole-2-carboxylic acid (Example 54);
benzyl 6-(2,4-dichlorophenyl)-1-hydroxy-4-(trifluoromethyl)-1H-indole-2-carboxylate (Example 55);
[1,1′-biphenyl]-4-ylmethyl 6-(2,4-dichlorophenyl)-1-hydroxy-4-(trifluoromethyl)-1H-indole-2-carboxylate (Example 56);
1-methylpiperidin-4-yl 1-hydroxy-6-phenyl-4-(trifluoromethyl)-1H-indole-2-carboxylate (Example 57);
1-methylpiperidin-4-yl 6-(2,4-dichlorophenyl)-1-hydroxy-4-(trifluoromethyl)-1H-indole-2-carboxylate (Example 58);
1-benzylpiperidin-4-yl 6-(2,4-dichlorophenyl)-1-hydroxy-4-(trifluoromethyl)-1H-indole-2-carboxylate (Example 59);
4-methoxybenzyl 6-(2,4-dichlorophenyl)-1-hydroxy-4-(trifluoromethyl)-1H-indole-2-carboxylate (Example 60);
4-nitrobenzyl 6-(2,4-dichlorophenyl)-1-hydroxy-4-(trifluoromethyl)-1H-indole-2-carboxylate (Example 61);
4-fluorobenzyl 6-(2,4-dichlorophenyl)-1-hydroxy-4-(trifluoromethyl)-1H-indole-2-carboxylate (Example 62);
4-chlorobenzyl 6-(2,4-dichlorophenyl)-1-hydroxy-4-(trifluoromethyl)-1H-indole-2-carboxylate (Example 63); and
4-(trifluoromethyl)benzyl 6-(2,4-dichlorophenyl)-1-hydroxy-4-(trifluoromethyl)-1H-indole-2-carboxylate (Example 64).

20. A method of treatment of cancer comprising administering to a subject in need thereof an effective amount of at least one compound as defined in claim 17.

21. The method according to claim 20, wherein the cancer is selected from the group consisting of:
lung tumor;
breast cancer;
cervical cancer; and
ovarian cancer.

22. The method according to claim 21 wherein the lung cancer is a non small cell lung carcinoma.

23. A method for the treatment of asthma, pulmonary hypertension, idiopathic arthrofibrosis, malaria, chronic back, or of hyperoxaluria comprising administering in a subject in need thereof an effective amount of at least one compound as defined in claim 17 to a patient in need thereof.

24. A process for the preparation of the compounds as defined in claim 1 comprising the steps as indicated in scheme 1, 2, 3, 4 and/or 5.
